(19) United States
(12) Patent Application Publication
(10) Pub. No.: US 2025/0320246 A1
(43) Pub. Date: Oct. 16, 2025

(54) 3-AZASTEROID COMPOUNDS FOR THE TREATMENT OF DISEASES RELATED TO MITROCHONDRIAL FUNCTION

(71) Applicant: NZP UK LIMITED, Bristol (GB)

(72) Inventors: Alexander Charles WEYMOUTH-WILSON, Bristol (GB); Laura Louise WALLIS, Bristol (GB); Gemma Louise PARKER, Bristol (GB); Heather MORTIBOYS, Sheffield (GB); Oliver BANDMANN, Sheffield (GB); Christopher HASTINGS, Sheffield (GB); Louise Danielle HEYWOOD, Sheffield (GB); Andreas LUXENBURGER, Wellington (NZ); Lawrence Daniel HARRIS, Wellington (NZ)

(21) Appl. No.: 18/870,074

(22) PCT Filed: Jun. 1, 2023
(86) PCT No.: PCT/GB2023/051450

§ 371 Date: Nov. 27, 2024

(30) Foreign Application Priority Data
Jun. 1, 2022 (GB) 2208151.7

(51) Int. Cl. C07J 73/00

(52) U.S. Cl. CPC C07J 73/005

(57) ABSTRACT

Compounds of formula (I):

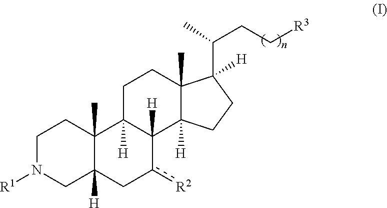

wherein R¹, R² and R³ are as defined herein are able to rescue dysfunctional mitochondria and are therefore of use in the treatment and prevention of neurodegenerative disorders as well as acute radiation syndrome and myalgic encephalomyelitis (ME, chronic fatigue syndrome) or post viral syndrome, including chronic symptoms arising from infection with SARS-CoV2 (long COVID).

The present invention relates to novel compounds which are of use in the treatment of neurodegenerative disorders and other conditions in which mitochondrial dysfunction is implicated and/or conditions in which modulating mitochondrial function is useful. In particular, the invention relates to bile acid derivatives, to pharmaceutical compositions containing them, process for preparing them and to the use of the compounds in the treatment or prevention of neurodegenerative disorders.

### BACKGROUND OF THE INVENTION

Neurodegenerative diseases are a group of disorders of the central nervous system and include Parkinson's disease, mild cognitive impairment, dementia (including Alzheimer's disease, vascular dementia and dementia with Lewy bodies), Huntington's disease and amyotrophic lateral sclerosis (motor neurone disease). The incidence of neurodegenerative disease increases with age and therefore such conditions are a growing problem in societies where the average age of the population is increasing. There is currently no cure for any of these diseases although there are some medications available which alleviate the symptoms of Parkinson's disease, some types of cognitive impairment and dementia.

The symptoms of Parkinson's disease are resting tremor, bradykinesia and rigidity and these symptoms are caused by neurodegeneration and loss of dopaminergic neurons. There is a large body of evidence which suggests that there is a strong association between mitochondrial dysfunction and Parkinson's disease. A mild deficiency of mitochondrial electron transport chain NADH dehydrogenase (complex I) activity has been found in the tissues of Parkinson's disease patients and a number of the proteins that are linked to the familial form of Parkinson's disease are either mitochondrial proteins or are associated with mitochondria.

Alzheimer's disease leads to progressive cognitive impairment and is characterised by the presence of extracellular neuritic plaques and intracellular neurofibrillary tangles. It is thought that mitochondrial dysfunction leads to the deposition of the β-amyloid proteins which are the major component of the neuritic plaques and to the formation of the neurofibrillary tangles.

Huntington's disease is an inherited progressive neurodegenerative disease and is characterised by motor impairment, personality changes and cognitive decline. The pathology of Huntington's disease provides evidence for a link with mitochondrial dysfunction.

Amyotrophic lateral sclerosis is also thought to be linked to mitochondrial dysfunction. This disease targets motor neurons in the central nervous system resulting in muscle weakness, atrophy and, death within 2-3 years of diagnosis.

Attempts have been made to find compounds which are capable of treating neurodegenerative disorders and several compounds have been developed which target mitochondria. For example, it is known that bile acids such as UDCA (ursodeoxycholic acid) exert a beneficial effect on mitochondrial dysfunction in tissue from certain patients suffering from Parkinson's disease, in particular in tissue from parkin mutant Parkinson's disease patients (Mortiboys, et al 2013) and LRRK2^{G2019S} mutant Parkinson's disease patients (Mortiboys et al 2015). Furthermore it is known bile acids such as UDCA exert a beneficial effect on fibroblasts from patients suffering from both sporadic Alzheimer's Disease and familial Alzheimer's Disease due to PSEN1 mutations (Bell et al 2018). Furthermore, additional studies have shown that UDCA is beneficial to cells from sporadic Parkinson's patients (Carling et al 2020).

WO 2014/036379, WO 2015/061421 and WO 2016/145216 teach that bile acids may be of use in the treatment of neurodegenerative disorders such as Parkinson's disease, Alzheimer's disease, Huntington's disease and amyotrophic lateral sclerosis. WO 2015/061421 relates to deuterated bile acids and WO 2016/145216 to fluorinated bile acids particularly bile acids fluorinated at the 3- and/or 7-positions. WO 2020/128514 relates to 2-fluorinated bile having mitochondrial rescue properties.

Mitochondrial dysfunction is also thought to play a role in acute radiation syndrome (ARS) since mitochondria are sensitive to oxidative stress. There is also evidence that spaceflight leads to altered mitochondrial function and DNA damage (da Silveira et al 2020). Mhatre et al 2022 considered the effects of the environment to which astronauts may be exposed during spaceflight and noted that exposure of mice to radiation was shown to lead to a number of effects such as increased lipid peroxidation and protein oxidation markers as well as mitochondrial damage, but that pre-treatment of mice with the antioxidant MitoQ mitigated this oxidative stress. Compounds which are able to rescue mitochondria may therefore be of use in the treatment of and prevention of ARS, both in the context of a nuclear accident or incident or in the exposure of a human or animal to radiation when undertaking space travel.

Mitochondrial dysfunction is also implicated in conditions such as myalgic encephalomyelitis (ME, chronic fatigue syndrome) and chronic symptoms arising from infection with SARS-CoV2 (long COVID) (Wood et al, 2021).

It would therefore be advantageous to develop further compounds which are able to rescue dysfunctional mitochondria.

### SUMMARY OF THE INVENTION

In a first aspect of the present invention there is provided a compound of formula (I):

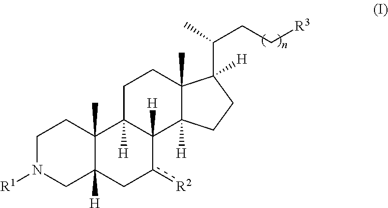

wherein:
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(Ra)(R^{11b});
   wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
      R^{14a} and R^{14b} are each independently selected from H and C₁₋₆ alkyl; and
   wherein each of R^{11a} and R^{11b} is independently selected from H and C₁₋₄ alkyl
R² is selected from O and OH, wherein when R² is O, [symbol] is a double bond and when R² is OH, [symbol] is a single bond;
R³ is selected from C(O)OH, C(O)OR¹⁶, C(O)N(R⁶)—X¹—R⁷, C(O)N(R⁸)(R⁹) and C(O)S—R¹⁰;
   R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
   wherein R^{15a} and R^{15b} are each independently selected from H, C₁₋₆ alkyl and C₁₋₆ haloalkyl;
   X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
      R^{12a} and R^{12b} are each independently selected from H and C₁₋₆ alkyl;
      R^{13a} and R^{13b} are each independently selected from H, C₁₋₆ alkyl and C₁₋₆ haloalkyl;
   R⁶ is selected from H, methyl and ethyl;
   R⁷ is selected from C(O)OH, C(O)O—(C₁₋₆ alkyl), S(O)₂OH, and S(O)₂—O—(C₁₋₆ alkyl);
   R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
      wherein R^{19a} and R^{19b} are each independently selected from H and C₁₋₆ alkyl;
   R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
   wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
      wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
   wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
      wherein R^{15a} and R^{15b} are each independently selected from H, C₁₋₆ alkyl and C₁₋₆ haloalkyl; or
   R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}). OH, O—(C₁₋₄ alkyl) or O—(C₁₋₄ haloalkyl);
      wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
   R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
      wherein R^{15a} and R^{15b} are each independently as defined above;
      wherein alkyl groups are optionally substituted by one or more groups selected from OH and C₃₋₆ cycloalkyl;
   wherein when the heteroaryl group contains a non-aromatic ring, the non-aromatic ring may also be substituted with oxo;
   R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
      wherein R^{15a} and R^{15b} are each independently as defined above; and
n is 1 or 2;
or a salt or solvate thereof.

### DETAILED DESCRIPTION OF THE INVENTION

In the present specification, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All publications, including but not limited to patents and patent applications, cited in this specification are herein incorporated by reference as if each individual publication were specifically and individually indicated to be incorporated by reference herein as though fully set forth.

In the present specification, references to “pharmaceutical use” refer to use for administration to a human or an animal, in particular a human or a mammal, for example a domesticated or livestock mammal, for the treatment or prophylaxis of a disease or medical condition. The term “pharmaceutical composition” refers to a composition which is suitable for pharmaceutical use and “pharmaceutically acceptable” refers to an agent which is suitable for use in a pharmaceutical composition. Other similar terms should be construed accordingly.

In the present application, the term “C₁₋₈” alkyl refers to a straight or branched fully saturated hydrocarbon group having from 1 to 8 carbon atoms. The term encompasses methyl, ethyl, n-propyl, isopropyl, n-butyl, isobutyl, s-butyl and t-butyl. Other alkyl groups, for example C₁₋₆ alkyl, C₁₋₄ alkyl, C₁₋₃ alkyl, or C₁₋₂ alkyl are as defined above but contain different numbers of carbon atoms.

The term “alkylene” refers to a straight or branched fully saturated hydrocarbon chain. Suitably alkylene is C₁₋₆ alkylene, C₁₋₅ alkylene, C₁₋₄ alkylene, C₁₋₃ alkylene, or C₁₋₂ alkylene. Examples of alkylene groups include —CH₂—, —CH₂CH₂—, —CH(CH₃)—CH₂—, —CH₂CH(CH₃)—, —CH₂CH₂CH₂—, —CH₂CH(CH₂CH₃)— and —CH₂CH(CH₂CH₃)CH₂—.

The term “C₂₋₆ alkenyl” refers to a straight or branched hydrocarbon group having from 2 to 6 carbon atoms and containing at least one carbon-carbon double bond. The term encompasses straight chain alkenyl groups such as CH═CH₂, CH₂CH═CH₂, CH═CHCH₃, CH₂CH₂CH═CH₂, CH═CHCH₂CH₃, CH₂CH═CHCH₃, CH₂CH₂CH₂CH═CH₂, CH═CHCH₂CH₂CH₃, CH₂CH═CHCH₂CH₃, CH₂CH₂CH═CHCH₃, CH═CHCH═CHCH₃ and CH₂CH═CHCH═CH₂, as well as branched alkenyl groups such as CH(CH₃)CH═CH₂ and CH═C(CH₃)CH₃. Other alkenyl groups, for example C₂₋₄ alkenyl, C₂₋₃ alkenyl and C₃₋₄ alkenyl are as defined above but contain different numbers of carbon atoms.

The term “C₂₋₆ alkynyl” refers to a straight or branched hydrocarbon group having from 2 to 6 carbon atoms and containing at least one carbon-carbon triple bond. The term encompasses straight chain alkenyl groups such as C≡CH, CH₂CH≡CH, C≡CCH₃, CH₂CH₂C≡CH, C≡CCH₂CH₃, CH₂C≡CCH₂CH₃, and CH₂C≡CCH═CH₂, as well as branched alkenyl groups such as CH(CH₃)—C≡CH. Other alkynyl groups, for example C₂₋₄ alkynyl, C₂₋₃ alkynyl and C₃₋₄ alkynyl are as defined above but contain different numbers of carbon atoms.

The term “halogen” refers to fluorine, chlorine, bromine or iodine and the term “halo” to fluoro, chloro, bromo or iodo groups.

The term “C₁₋₆ haloalkyl” refers to a straight or branched alkyl group as defined above having from 1 to 6 carbon atoms and substituted with one or more halo atoms, up to perhalo substitution. Examples include trifluoromethyl, chloroethyl and 1,1-difluoroethyl. Other haloalkyl groups, for example C₁₋₅ haloalkyl, C₁₋₄ haloalkyl, C₁₋₃ haloalkyl or C₁₋₂ haloalkyl are as defined above but contain different numbers of carbon atoms.

The terms “aryl” and “aromatic” refer to a cyclic group with aromatic character having from 6 to 14 ring carbon atoms (unless otherwise specified, for example 6 to 10 ring carbon atoms) and containing up to three rings. Where an aryl group contains more than one ring, not all rings must be aromatic in character. Examples include phenyl, naphthyl and anthracenyl as well as partially saturated systems such as tetrahydronaphthyl (e.g. 1,2,3,4-tetrahydronaphthyl), indanyl and indenyl.

The terms “heteroaryl” and “heteroaromatic” refer to a cyclic group with aromatic character having from 5 to 14 ring atoms (unless otherwise specified, for example 5 to 10 ring atoms), containing at least one heteroatom selected from N, O and S and comprising up to three rings. Where a heteroaryl group contains more than one ring, not all rings must be aromatic in character. Examples include pyridine, pyrimidine, pyrrole, thiophene, furan, thiazole, oxazole, fused systems such as indole, benzimidazole and benzothiophene; and partially saturated systems such as indoline, isoindoline and dihydrobenzofuran.

The terms “carbocyclic” and “carbocyclyl” refer to a non-aromatic hydrocarbon ring system having from 3 to 10 ring carbon atoms (unless otherwise specified) and containing up to 3 rings, which may be fused or joined by a spiro linkage or be a bridged ring system. A carbocyclic group optionally comprises one or more carbon-carbon double bonds. Examples include cycloalkyl groups such as cyclopropyl, cyclobutyl, cyclopentyl, cyclohexyl, cycloheptyl and cyclooctyl; and cycloalkenyl groups such as cyclohexenyl, and cycloheptenyl; and bridged groups such as adamantyl. More suitably, the carbocyclyl group is a monocylic fully saturated (cycloalkyl) ring.

The terms “heterocyclic” and “heterocyclyl” refer to a non-aromatic ring system having from 3 to 10 ring carbon atoms (unless otherwise specified), and at least one heteroatom selected from N, O and S and containing up to three rings, which may be fused, joined by a spiro linkage or be a bridged ring system. A heterocyclic group may be fully saturated or may comprise one or more carbon-carbon or carbon-nitrogen double bonds. Examples include piperidinyl, morpholinyl, thiomorpholinyl, thiozolidinyl, tetrahydrothiophenyl and tetrahydrothiopyranyl. More suitably, the heterocyclyl group is a monocylic fully saturated ring.

The term “oxo” refers to a carbonyl substituent (═O), especially on a carbocyclic or heterocyclic ring. A ring carbon atom may be substituted with one oxo group and a ring sulfur atom may be substituted with one or two oxo groups.

The term “protected NH₂ group” refers to an amine protected by any known protecting group. Examples of protected NH₂ groups include carbamates such as benzyl carbamate (carboxybenzyl, NHCBz), t-butyl carbamate (NHBoc) and 9-fluorenylmethylcarbamate (NHFmoc). Other suitable protecting groups for NH₂ include triphenylmethyl (trityl), acetyl, benzyl and paramethoxybenzyl. Other protecting groups for NH₂ are well known to those of skill in the art (see e.g. Wuts, P G M and Greene, TW (2006) “Greene's Protective Groups in Organic Synthesis”, 4^{th} Edition, John Wiley & Sons, Inc., Hoboken, NJ, USA).

The term “protected OH group” refers to a hydroxyl protected by any known protecting group. Examples of protected OH groups of this type include R¹⁸C(O)O, where R¹⁸ is C₁₋₆ alkyl or benzyl, especially methyl. Silyl ether protecting groups may also be used and OH can also be protected as an ether, for example a C₁₋₆ alkyl, benzyl or p-methoxybenzyl ether. Other suitable protecting groups for OH are well known to those of skill in the art (see e.g. Wuts, P G M and Greene, TW (2006) “Greene's Protective Groups in Organic Synthesis”, 4^{th} Edition, John Wiley & Sons, Inc., Hoboken, NJ, USA).

Salts of the compounds of formula (I) may be acid addition salts of the quaternary amine formed when the nitrogen atom attached to R¹ is quaternised. Alternatively, when R³ comprises a C(O)OH or S(O)₂OH, the salt may be a basic addition salt. When R¹ comprises a substituent N(R^{11a})(R^{11b}) or N(R^{14a})(R^{14b}) or when R³ comprises a substituent N(R^{12a})(R^{12b}), N(R^{13a})(R^{13b}), N(R^{15a})(R^{15b}) or N(R^{19a})(R^{19b}) or when R⁴ comprises an amine group, a salt may be formed by quaternisation of the amine.

Any salts intended to be administered to a patient will be pharmaceutically acceptable but other salts may also be used during the synthesis of a pharmaceutically acceptable final product. Pharmaceutically acceptable salts are known to those of skill in the art and are summarised in Gupta et al, Molecules, 23, 1719 (2018).

Pharmaceutically acceptable acid addition salts include hydrochloride, trifluoroacetate, mesylate, hydrobromide, sulphate, and fumarate salts.

Pharmaceutically acceptable basic addition salts include sodium, potassium, calcium, aluminium, zinc, magnesium and other metal salts as well as choline, amine salts including triethylamine, N,N-diisopropylethylamine (DIPEA), diethanolamine, ethanolamine, ethyl diamine, meglumine and other well-known basic addition salts.

The compounds of formula (I) include all stereoisomers. In the compounds of the invention, the stereochemistry of the bile acid ring system is fixed and therefore the term “stereoisomers” as used herein refers only to stereoisomers of the R¹ and/or the R³ substituents in the compounds of formula (I) and not to stereoisomers of the bile acid ring system.

The compounds of formula (I) include all isotopic variants. The term “isotopic variant” refers to isotopically-labelled compounds which are identical to those recited in formula (I) but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”). Examples of isotopes that can be incorporated into compounds of the invention include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I (e.g ³H, ¹¹C, ¹⁴C, ¹⁸F, ¹²³I or ¹²⁵I), which may be naturally occurring or non-naturally occurring isotopes.

As noted above, the invention provides a compound of formula (I) as defined above or a salt, solvate and/or isotopic variant thereof.

In a further aspect of the invention there is provided a compound of formula (IZ):

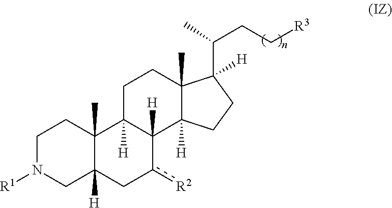

wherein:
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
   wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a protected OH group, a protected NH₂ group, a protected C(O)NH₂ group, a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
      R^{14a} and R^{14b} are each independently selected from H and C₁₋₆ alkyl; and wherein each of R^{11a} and R^{11b} is independently selected from H and C₁₋₄ alkyl
R² is selected from O and OH, wherein when R² is O, [symbol] is a double bond and when R² is OH, [symbol] is a single bond;
R³ is selected from C(O)OH, C(O)OR¹⁶, C(O)N(R)—X¹—R⁷, C(O)N(R⁸)(R⁹) and C(O)S—R¹⁰;
   R¹⁶ is selected from C₁₋₈ alkyl and benzyl;
   X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}), a protected OH group, a protected NH₂ group, a protected C(O)NH₂ group and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
      R^{12a} and R^{12b} are each independently selected from H and C₁₋₆ alkyl;
      R^{13a} and R^{13b} are each independently selected from H, C₁₋₆ alkyl and C₁₋₆ haloalkyl;
   R⁶ is selected from H, methyl and ethyl;
   R⁷ is selected from C(O)OH, C(O)O—(C₁₋₆ alkyl), S(O)₂OH, and S(O)₂O—(C₁₋₆ alkyl);
   R⁸ is selected from H, C₁₋₆ alkyl and a 3- to 6-membered carbocyclyl ring optionally substituted with one or more substituents selected from C₁₋₃ alkyl, OH, O—(C₁₋₃ alkyl) and halo;
   R⁹ is selected from C₁₋₆ alkyl, a 5- or 6-membered carbocyclyl ring optionally substituted with one or more substituents selected from C₁₋₃ alkyl, OH, O—(C₁₋₃ alkyl) and halo, and phenyl optionally substituted with one or more substituents selected from halo, NO₂, CN, S(O)₂OH, C₁₋₆ alkyl, O(R^{15a}), N(R^{15a})(R^{15b}), C(O)OH and C(O)O—(C₁₋₄ alkyl);
      wherein R^{15a} and R^{15b} are each independently selected from H and C₁₋₆ alkyl; or
   R⁸ and R⁹ together with the nitrogen atom to which they are attached combine to form a 5- or 6-membered heterocyclic ring, optionally containing one or more further heteroatoms selected from O, N and S and optionally substituted with one or more substituents selected from C₁₋₄ alkyl, OH, O—(C₁₋₃ alkyl), halo, C(O)OH and C(O)O(C₁₋₄ alkyl);
   R¹⁰ is C₁₋₆ alkyl optionally substituted with OH, halo or phenyl, wherein phenyl is optionally substituted with one or more substituents selected from halo, OH, C₁₋₄ alkyl, C₁₋₄ haloalkyl, O(C₁₋₄ alkyl) and O(C₁₋₄ haloalkyl);
n is 1 or 2;
or a salt or solvate thereof.

In some cases, [symbol] is a single bond and the compound of formula (I) or (IZ) is a compound of formula (IA) or (IB):

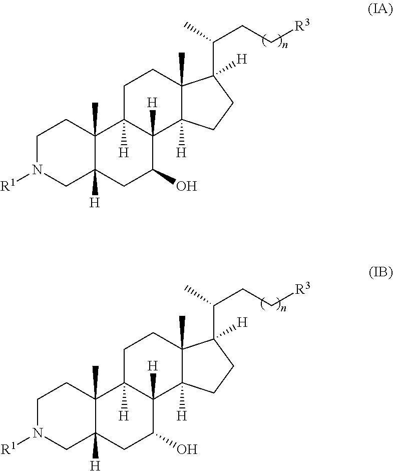

Wherein R¹, R³ and n are as defined above for formula (I) or formula (IZ).

Alternatively, [symbol] is a double bond and the compound of formula (I) or (IZ) is a compound of formula (IC):

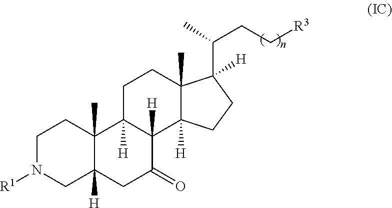

wherein R¹, R³ and n are as defined above for formula (I) or formula (IZ).

In some suitable compounds, the compound of formula (I) or formula (IZ) is a compound of formula (IA).

In some suitable compounds, the compound of formula (I) or formula (IZ) is a compound of formula (IB).

In some suitable compounds, the compound of formula (I) or formula (IZ) is a compound of formula (IC).

In some suitable compounds of the invention, R¹ is H, C₁₋₆ alkyl, C₂₋₆ alkenyl or C₂₋₆ alkynyl, more suitably H or C₁₋₆ alkyl, especially H, methyl or ethyl. In particularly suitable compounds, R¹ is H.

In other suitable compounds of the invention, R¹ is —C(O)R⁴, wherein R⁴ is as defined above for formula (I) and formula (IZ).

In some more suitable compounds of formula (I), R¹ is —C(O)R⁴ and R⁴ is selected from C₁₋₆ alkyl optionally substituted with one or more substituents selected from OH, NH₂, NH₃+ and a 6- to 14-membered aryl or 5- to 14-membered heteroaryl wherein the aryl and heteroaryl are optionally substituted with one or more substituents selected from OH and halo. In more suitable compounds of this type, R⁴ is selected from C₁₋₆ alkyl optionally substituted with one or more substituents selected from OH, NH₂, NH₃⁺, phenyl optionally substituted with one or more substituents selected from OH and halo, and a 5- to 10-membered nitrogen-containing heteroaryl group such as pyrrole, pyridine or indole, optionally substituted with one or more substituents selected from OH and halo.

In particularly suitable compounds of formula (I) in which R¹ is —C(O)R⁴, R¹ is an amino acid residue or salt thereof. In the present specification, “amino acid residue” refers to an amino acid which lacks the OH group, i.e. a substituent of the type —C(O)—C(R)—NH₂, where R is an amino acid side chain. A salt of an amino acid residue is a substituent of the type —C(O)—C(R)—NH₃+. During synthesis of such compounds, an N-protected amino acid residue may be used as an intermediate. An N-protected amino acid residue is a substituent of the type —C(O)—C(R)—NHP¹, where P¹ is an amine protecting group. Suitable amine protecting groups P¹ are as discussed above but are particularly carbamate-forming groups such as —C(O)O-t-butyl (Boc), —C(O)O-fluorenylmethyl (Fmoc) and —C(O)O-benzyl (CBz).

In compounds of formula (I), examples of R¹ groups of this type include residues of glycine, valine, isoleucine, leucine, tryptophan and tyrosine and salts of these amino acid residues.

In some suitable compounds of formula (I), R¹ is —C(O)OR⁵ wherein R⁵ is as defined above for formula (I) or formula (IZ).

In more suitable compounds of formula (I) in which R¹ is —C(O)OR⁵, R⁵ is selected from C₁₋₆ alkyl optionally substituted with one or more substituents selected from OH, NH₂, NH₃+, a 6- to 14-membered aryl and a 5- to 14-membered heteroaryl wherein the aryl and heteroaryl are optionally substituted as described above for formula (I) or formula (IZ).

In some still more suitable compounds of formula (I), R⁵ is C₁₋₆ alkyl (e.g. t-butyl), benzyl or fluorenylmethyl.

In particularly suitable compounds of this type, R¹ is C(O)O-benzyl (CBz).

In some suitable compounds of formula (IZ) in which R¹ is —C(O)R⁴, R⁴ is selected from C₁₋₆ alkyl optionally substituted with one or more substituents selected from OH, NH₂, NH₃⁺, a protected OH group, a protected NH₂ group and a 6- to 14-membered aryl or 5- to 14-membered heteroaryl wherein the aryl and heteroaryl are optionally substituted with one or more substituents selected from OH and halo. In more suitable compounds of this type, R⁴ is selected from C₁₋₆ alkyl optionally substituted with one or more substituents selected from OH, NH₂, NH₃⁺, a protected NH₂ group, phenyl optionally substituted with one or more substituents selected from OH and halo, and a 5- to 10-membered nitrogen-containing heteroaryl group such as pyrrole, pyridine or indole, optionally substituted with one or more substituents selected from OH and halo.

In particularly suitable compounds of formula (IZ) in which R¹ is —C(O)R⁴, R¹ is an N-protected amino acid residue, an amino acid residue or salt thereof. In the present specification, “amino acid residue” refers to an amino acid which lacks the OH group, i.e. a substituent of the type —C(O)—C(R)—NH₂, where R is an amino acid side chain. A salt of an amino acid residue is a substituent of the type —C(O)—C(R)—NH₃⁺, and an N-protected amino acid residue is a substituent of the type —C(O)—C(R)—NHP¹, where P¹ is an amine protecting group. Suitable amine protecting groups P¹ are as discussed above but are particularly carbamate-forming groups such as —C(O)O-t-butyl (Boc), —C(O)O-fluorenylmethyl (Fmoc) and —C(O)O-benzyl (CBz).

In compounds of formula (IZ), examples of R¹ groups of this type include residues of glycine, valine, isoleucine, leucine, tryptophan and tyrosine, salts of these amino acid residues and N-protected residues of glycine, valine, isoleucine, leucine, tryptophan and tyrosine.

In other suitable compounds formula (IZ), R¹ is —C(O)OR⁵, wherein R⁵ is selected from C₁₋₆ alkyl optionally substituted with one or more substituents selected from OH, NH₂, NH₃⁺, a protected OH group, a protected NH₂ group and a 6- to 14-membered aryl or 5- to 14-membered heteroaryl wherein the aryl and heteroaryl are optionally substituted as described above. In more suitable compounds of this type, C(O)OR⁵ is an amine protecting group and forms a carbamate with the nitrogen atom to which it is linked. R⁵ is still more suitably C₁₋₆ alkyl optionally substituted with a 6- to 14-membered aryl or 5- to 14-membered aryl group. For example, R⁵ may be t-butyl, benzyl or fluorenylmethyl.

Some compounds of formulae (I), (IZ), (IA), (IB) and (IC) are salts in which the nitrogen atom to which R¹ is linked is quaternised such that the compound is of formula (ID), (IE), (IF) or (IG):

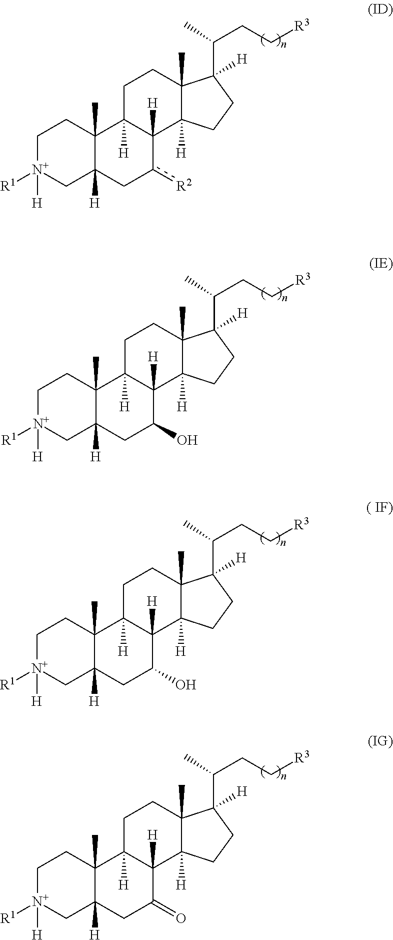

wherein R¹, R² and R³ are as defined above for formula (I) or formula (IZ).

In the embodiments directly above, a counterion (anion) Z⁻ is also present. Suitable counterions include chloride, trifluoroacetate, mesylate, bromide, sulphate, and fumarate salts, especially chloride and trifluoracetate.

In some suitable compounds, the compound of formula (I) or formula (IZ) is a compound of formula (ID).

In some suitable compounds, the compound of formula (I) or formula (IZ) is a compound of formula (IE).

In some suitable compounds, the compound of formula (I) or formula (IZ) is a compound of formula (IF).

In some suitable compounds, the compound of formula (I) or formula (IZ) is a compound of formula (IG).

Suitable counter ions for such salts include acid addition salts, especially the pharmaceutically acceptable acid addition salts discussed above.

In some compounds of formulae (I) and formula (IZ), n is 1 and in other compounds of formulae (I), n is 2.

As noted above, in the compounds of formula (I) and formula (IZ), R³ is selected from C(O)OH, C(O)OR¹⁶, C(O)N(R⁶)—X¹—R⁷, C(O)N(R⁸)(R⁹) and C(O)S—R¹⁰.

In some suitable compounds of the invention, R³ is C(O)OH or C(O)OR¹⁶, where R¹⁶ is C₁₋₈ alkyl or benzyl. In some cases, R³ is C(O)OH or C(O)(C₁₋₆ alkyl), for example C(O)OH or C(O)O(C₁₋₄ alkyl) and especially C(O)OH.

In other suitable compounds of the invention, R³ is C(O)N(R⁶)—X¹—R⁷, wherein R⁶, X¹ and R⁷ are as defined above for formula (I) or formula (IZ). Suitably in these compounds, R⁶ is H or methyl. In some embodiments R⁶ is H and in other embodiments R⁶ is methyl.

As noted above, X¹ is C₁₋₆ alkylene optionally substituted as described above for formula (I) or formula (IZ). In some cases, the alkylene group is a straight chain alkylene and in other cases, the alkylene group is a branched chain alkylene.

In some cases, X¹ is unsubstituted and in other cases X¹ is substituted as defined above for formula (I) or formula (IZ). In compounds of formula (I) and formula (IZ), suitable substituents for X¹ include halo, OR^{12a}, SR^{12a}, N(R^{12a})(R^{12b}) C(O)OR^{12a}, phenyl and 5- or 6-membered heteroaryl, wherein phenyl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl; and
R^{13a} and R^{13b} are each independently selected from H, C₁₋₆ alkyl and C₁₋₆ haloalkyl.

More suitable substituents for X¹ include halo, OH, O(C₁₋₄ alkyl), SH, S(C₁₋₄ alkyl), C(O)OH, C(O)O—(C₁₋₆ alkyl), NH₂, NH(C₁₋₄ alkyl), N(C₁₋₄ alkyl)₂ and phenyl optionally substituted with one or more substituents selected from OH, halo, O(C₁₋₃ alkyl) and O(C₁₋₃ haloalkyl).

Some more suitable substituents for X¹ include halo, OH, O(C₁₋₄ alkyl), SH, S(C₁₋₄ alkyl), C(O)OH, C(O)O—(C₁₋₆ alkyl) and phenyl optionally substituted with one or more substituents selected from OH, halo, O(C₁₋₃ alkyl) and O(C₁₋₃ haloalkyl).

Other more suitable substituents for X¹ include halo, OH, O(C₁₋₄ alkyl), S(C₁₋₄ alkyl), NH₂ and phenyl optionally substituted with halo or OH.

Still more suitable substituents for X¹ include fluoro, OH, methoxy, ethoxy, i-propyloxy, s-butyloxy, t-butyloxy, S-methyl, NH₂, C(O)OH, phenyl and phenyl substituted with OH. Examples of particularly suitable substituents for X¹ include fluoro, OH, methoxy, ethoxy, i-propyloxy, t-butyloxy, S-methyl, phenyl and phenyl substituted with OH.

As noted above, R⁷ is selected from C(O)OH, C(O)O—(C₁₋₆ alkyl), S(O)₂OH, and S(O)₂—O—(C₁₋₆ alkyl). More suitably R⁷ is selected from C(O)OH, C(O)O—(C₁₋₃ alkyl) and S(O)₂OH, especially C(O)OH and S(O)₂OH.

Particularly suitable groups C(O)N(R⁶)—X¹—R⁷ include C(O)NH—(CH₂)₂—SO₂OH (taurine conjugate) and C(O)NH—CH₂—C(O)OH (glycine conjugate). Other suitable amino acid conjugates include conjugates with O-t-butyl-L-serine (R³ is C(O)NH—CH(CH₂O^{t}Bu)-C(O)OH), β-phenylalanine (R³ is C(O)NH—CH(Ph)-CH₂—C(O)OH), serine (R³ is C(O)NH—CH(CH₂OH)—C(O)OH), 3-amino-2-fluoropropionic acid (R³ is C(O)NH—CH₂CHF—C(O)OH), methionine (R³ is C(O)NH—CH(CH₂CH₂SMe)-C(O)OH, β-alanine (R³ is C(O)NH—CH₂CH₂—C(O)OH), valine (R³ is C(O)NH—CH(ⁱPr)—C(O)OH), isoleucine (R³ is C(O)NH—CH(CH[Me]CH₂Me)-C(O)OH), sarcosine (R³ is C(O)N(Me)-CH₂—C(O)OH), alanine (R³ is C(O)NH—CH(Me)-C(O)OH, aspartic acid (R³ is C(O)NH—CH(CH₂C(O)OH)—C(O)OH, phenylalanine (R³ is C(O)NH—CH(CH₂Ph)-C(O)OH, 3-aminobutanoic acid (R³ is C(O)NH—CH(Me)CH₂—C(O)OH), leucine (R³ is C(O)NH—CH(^{t}Bu)-C(O)OH), lysine (R³ is C(O)NH—(CH₂)₄—CH(NH₂)—C(O)OH) and tyrosine (R³ is C(O)NH—C(CH₂Ph-OH)—C(O)OH).

Suitably, the amino acid with which the conjugate is formed is in the L configuration.

In some compounds of formula (I) and formula (IZ), R³ is C(O)N(R⁸)(R⁹), where R⁸ and R⁹ are as defined above for formula (I) or formula (IZ).

In some suitable compounds of this type, R⁸ is selected from H, C₁₋₄ alkyl, cyclopentyl or cyclohexyl, wherein cyclopentyl and cyclohexyl groups are optionally substituted with methyl, OH, methoxy or fluoro but are more suitably unsubstituted.

More suitably, R⁸ is selected from H, methyl, ethyl, unsubstituted cyclopentyl and unsubstituted cyclohexyl, for example H, methyl or unsubstituted cyclohexyl and especially H.

As noted above, when R³ is C(O)N(R⁸)(R⁹), R⁹ may be H, C₁₋₆ alkyl, a 3- to 7-membered carbocyclyl group, a 3- to 7-membered heterocyclyl group, 6- to 14-membered aryl or 5- to 14-membered heteroaryl, and, in particular, R⁹ is selected from C₁₋₆ alkyl, a 3- to 7-membered carbocyclyl group, a 3- to 7-membered heterocyclyl group, 6- to 14-membered aryl and 5- to 14-membered heteroaryl, wherein carbocyclyl, heterocyclyl, aryl and heteroaryl groups are optionally substituted as defined above for formula (I) or formula (IZ).

In some suitable compounds of the invention, R⁹ is C₁₋₆ alkyl, for example methyl. Alkyl groups R⁹ may be unsubstituted or substituted as described above. For example, in some compounds of formula (I), R⁹ is C₁₋₆ alkyl, such as methyl, which may be substituted with a 3- to 7-membered heterocyclyl group, especially a 5- or 6-membered heterocyclyl group such as morpholinyl, piperidinyl, piperazinyl, pyrrolidinyl or tetrahydrofuryl wherein the heterocyclyl group may be unsubstituted or substituted as described above for formula (I).

In some particularly suitable compound of formula (I) or (IZ), in which R³ is C(O)N(R⁸)(R⁹), R⁸ is H and R⁹ is C₁₋₆ alkyl, for example methyl, which is unsubstituted or substituted as described above for formula (I). For example, R⁸ is H and R⁹ is C₁₋₆ alkyl, such as methyl substituted with a 3- to 7-membered heterocyclyl group, especially a 5- or 6-membered heterocyclyl group such as morpholinyl, piperidinyl, piperazinyl, pyrrolidinyl ortetrahydrofuryl, more usually morpholinyl, piperidinyl or piperazinyl and especially morpholinyl, wherein the heterocyclyl group may be unsubstituted or substituted as described above for formula (I).

In some suitable compounds of formula (I) and formula (IZ), R⁹ is selected from a 3- to 7-membered carbocyclyl group, a 3- to 7-membered heterocyclyl group, phenyl or 5- or 6-membered heteroaryl, any of which is unsubstituted or substituted as defined above for formula (I) or formula (IZ).

When R⁹ is a carbocyclyl group it is more suitably cyclopentyl or cyclohexyl.

In some particularly suitable compounds of formula (I) or (IZ), in which R³ is C(O)N(R⁸)(R⁹), R⁸ and R⁹ are both 3- to 6-membered cycloalkyl rings, for example R⁸ and R⁹ are both cyclohexyl.

In other suitable compounds of formula (I), R⁹ is a heterocyclyl group. More suitably, R⁹ is a 5- or 6-membered heterocyclyl group, containing, 1 to 3, for example 1 or 2, heteroatoms, especially N and/or O. For example, R⁹ may be a nitrogen- and/or oxygen-containing heterocyclyl group such as pyrrolidinyl, tetrahydrofuranyl, piperidinyl, piperazinyl or morpholinyl, especially tetrohydrofuranyl. Heterocycyl groups may be unsubstituted or substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl), and especially suitable substituents include oxo. A substituent may be on a carbon atom and/or on a heteroatom selected from N and S (when present). In this case, the substituent on the N or S atom will not be OH, O—(C₁₋₄ alkyl) or O—(C₁₋₄ haloalkyl) and the substituent on the N atom will not be oxo.

In more suitable compounds of formula (I), R³ is C(O)N(R⁸)(R⁹), R⁸ is H and R⁹ is a heterocyclyl group, especially a more suitable heterocyclyl group which is unsubstituted or substituted as defined above for formula (I).

When R⁹ is a 6- to 14-membered aryl group, it may be, for example phenyl or naphthyl, and especially phenyl. An aryl group R⁹ may be unsubstituted or substituted as defined above for formula (I), especially with one or more substituents selected from halo, C(O)OH and C(O)O—(C₁₋₄ alkyl), for example fluoro, C(O)OH and C(O)O—(C₁₋₃ alkyl). In some cases, an aryl group R⁹ is unsubstituted or, more suitably, is substituted with a single substituent. When R⁹ is phenyl, a single substituent may be at the 4-position.

In some more suitable compounds of formula (I) in which R³ is C(O)N(R⁸)(R⁹), R⁸ is H and R⁹ is phenyl optionally substituted as defined above for formula (I).

When R⁹ is a heteroaryl group, it may be a 5- or 6-membered heteroaryl group, particularly a nitrogen-containing heteroaryl group such as pyridyl, for example pyridin-2-yl. The heteroaryl group may be substituted or unsubstituted and suitable substituents include halo, C(O)OH and C(O)O—(C₁₋₄ alkyl), for example fluoro, C(O)OH and C(O)O—(C₁₋₃ alkyl).

In some more suitable compounds of formula (I), in which R³ is C(O)N(R⁸)(R⁹), R⁸ is H and R⁹ is a 6- to 14-membered heteroaryl group, for example pyridyl, which is unsubstituted or substituted as defined above for formula (I).

In some compounds of formula (I) and formula (IZ), R³ is C(O)N(R⁸)(R⁹) and R⁸ and R⁹ together with the nitrogen atom to which they are attached combine to form a 4- to 10-membered heterocyclic group, for example a 5- to 7-membered heterocyclic group, or a 5- or 6-membered heterocyclic group optionally containing one or more further heteroatoms selected from O, N and S and optionally substituted with one or more substituents as defined above for formula (I) or formula (IZ).

In some suitable compounds, the heterocyclic group comprises a single ring, especially a 4- to 7-membered ring, for example a 5- or 6-membered ring. The ring may comprise no additional heteroatoms, such that the nitrogen atom to which R⁸ and R⁹ are attached is the only heteroatom in the heterocyclic group. Alternatively, the ring may comprise one or more additional heteroatoms, for example one additional heteroatom or two additional heteroatoms. The additional heteroatoms are suitably selected from N, O and S.

Example of single ring heterocyclic groups formed by R⁸ and R⁹ together with the nitrogen atom to which they are attached include morpholine, piperidine, piperazine, pyrrolidine, thiazoline, isothiazoline, thiazolidine, isothiazolidine, oxazoline, isooxazoline, oxazolidine, isoxazolidine, pyrazoline and pyrazolidine where the ring is unsubstituted or is substituted as defined above. More suitable examples of single ring heterocyclic groups formed by R⁸ and R⁹ together with the nitrogen atom to which they are attached include piperidine, pyrrolidine, piperazine, morpholine and isothiazolidine.

In other suitable compounds, the heterocyclic group formed by R⁸ and R⁹ and the nitrogen atom to which they are attached comprises two or more rings, especially two rings, which may be fused or bridged or joined by a spiro linkage. The heterocyclic group may comprise no additional heteroatoms, such that the nitrogen atom to which R⁸ and R⁹ are attached is the only heteroatom in the heterocyclic group. Alternatively, the heterocyclic group may comprise one or more additional heteroatoms, for example one additional heteroatom or two additional heteroatoms. The additional heteroatoms are suitably selected from N, O and S. In some cases each of the rings in the system may contain one or two heteroatoms.

Spiro linked groups may comprise a 4- or 5-membered ring containing the nitrogen atom to which R⁸ and R⁹ are attached linked via a spiro linkage to a 3- to 6-membered ring, especially to a 4- or 5-membered ring, optionally containing a further heteroatom selected from N, O and S, especially O. An example of such a system is 2-oxa-6-azospiro{3,3}heptane (i.e. an azetidine ring spiro linked at the 3-position to an oxetane ring).

Fused systems may comprise a 5- or 6-membered ring containing the nitrogen atom to which R⁸ and R⁹ are attached fused to a 3- to 6-membered ring.

Bridged systems may comprise a 5- or 6-membered ring containing the nitrogen atom to which R⁸ and R⁹ are attached with a bridge having one or two atoms, for example a bridge selected from —CH₂—, —CH₂CH₂—, —O—, —NH— and N(C₁₋₄ alkyl).

When R⁸ and R⁹ together with the nitrogen atom to which they are attached combine to form a heterocyclic group, this may be unsubstituted or substituted. In some compounds, especially compounds of formula (I), the substituents are selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), benzyl, N(R^{15a})(R^{15b}) (for example NH₂) and oxo, for example C₁₋₃ alkyl, OH, O—(C₁₋₃ alkyl), halo, benzyl, NH₂ and oxo. A substituent may be on a carbon atom and/or on a further heteroatom selected from N and S (when present). In this case, the substituent on the N or S atom will not be OH, O—(C₁₋₄ alkyl) or O—(C₁₋₄ haloalkyl).

A heterocyclic ring containing no further heteroatoms may have one or more oxo substituents on carbon atoms and particularly suitable substituents for ring carbon atoms include halo, C₁. 4 alkyl, C₁₋₄ haloalkyl, OH, O(C₁₋₄ alkyl), benzyl, N(R^{15a})(R^{15b}) (for example NH₂) and oxo. In particularly suitable compounds of this type, R⁸ and R⁹ together with the nitrogen atom to which they are attached may form a piperidone or a pyrrolidone ring, e.g. a 4-piperidone or a 3-pyrrolidone ring.

In a heterocyclic group containing one or more further nitrogen atoms, there may be a substituent on a ring nitrogen atom and suitable substituents for ring nitrogen atoms include C₁₋₄ alkyl, C₁₋₄ haloalkyl and benzyl. Such heterocyclic groups may contain no additional substituents or may contain one or more substituents on ring carbon atoms as described above.

A heterocyclic group containing one or more sulfur atoms may have one or more substituents, suitably oxo substituents, on ring sulfur atoms. A ring sulfur atom may have one or two oxo substituents. In addition, a heterocyclic group containing one or more sulfur atoms may contain no additional substituents or may contain one or more substituents on ring carbon atoms as described above. For example, such a ring may have a single oxo substituent on one or more carbon atoms and/or one or two oxo substituents on a sulfur atom.

In other compounds, especially compounds of formula (IZ), the substituents are selected from C₁₋₃ alkyl, OH, O—(C₁₋₃ alkyl) and halo.

In other suitable compounds in which R³ is C(O)N(R⁸)(R⁹), R⁸ and R⁹ together with the nitrogen atom to which they are attached combine to form a 5- to 10-membered heteroaryl group. The heteroaryl group optionally contains one or more further heteroatoms selected from N, O and S, and is either unsubstituted or is substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)OH and C(O)O—(C₁₋₆ alkyl).

In some cases, the heteroaryl group has a single 5- or 6-membered ring, for example a single 5-membered ring. For example, the heteroaryl group may be pyrrole, imidazole, triazole or thiazole, especially pyrrole.

In other cases, the heteroaryl group may have two rings. In some compounds of the invention, both rings are aromatic in character and the nitrogen atom to which R⁸ and R⁹ are attached is part of a 5-membered ring fused to a further aromatic or heteroaromatic ring. Examples of heteroaryl groups of this type include indole and isoindole, especially isoindole. Alternatively, one of the rings may be partially or fully saturated. Suitably in this case the saturated or partially saturated ring is the ring containing the nitrogen atom to which R⁸ and R⁹ are attached. The other ring of the heterocyclic group may be a 5- or 6-membered ring such as phenyl, pyridyl or pyrrolyl.

In other more suitable compounds of formula (I) or formula (IZ) in which R³ is C(O)N(R⁸)(R⁹), R⁸ and R⁹ together with the nitrogen atom to which they are attached combine to form a morpholine, piperidine or piperazine ring, where the ring is unsubstituted or is substituted as defined above.

Particularly suitable groups C(O)N(R⁸)(R⁹) in the compounds of formula (I) and formula (IZ) include those in which:
R⁸ is H and R⁹ is 4-fluorophenyl;
R⁸ and R⁹ are each cyclohexyl;
R⁸ is H and R⁹ is 4-benzoic acid or a C₁₋₄ alkyl ester thereof, e.g. isopropyl-4-benzoate;
R⁸ is H and R⁹ is tetrahydrofuranyl, especially tetrahydrofuran-3-yl;
R⁸ and R⁹ together with the N atom to which they are attached combine to form an isoindoline ring;
R⁸ and R⁹ together with the N atom to which they are attached combine to form a morpholine ring;
R⁸ and R⁹ together with the N atom to which they are attached combine to form a piperidine or pyrrolidine ring substituted with oxo, e.g. 4-piperidone or 3-pyrrolidone;
R⁸ and R⁹ together with the N atom to which they are attached combine to form a pyrrole ring.

In still other compounds of the invention, R³ is C(O)S—R¹⁰, where R¹⁰ is as defined above for formula (I) or formula (IZ) but is more suitably C₁₋₆ alkyl optionally substituted with OH, halo or phenyl. In still more suitable compounds of this type, R¹⁰ is C₁₋₄ alkyl optionally substituted with OH, halo or phenyl. In particularly suitable compounds, R¹⁰ is benzyl.

In some suitable compounds of the invention, R¹ and R² are as defined above and R³ is C(O)OH or C(O)OR¹⁶, wherein R¹⁶ is as defined above for formula (I) or formula (IZ).

In some particularly suitable compounds of the invention, R² is OH and R³ is C(O)OH and the compound is of formula (IH) or a salt of formula (IJ):

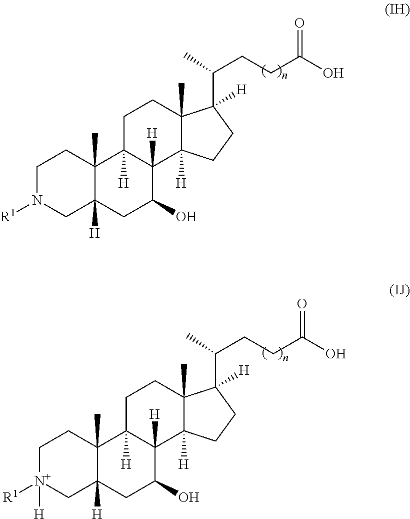

where R¹ is as defined above for formula (I) or formula (IZ).

In some compounds of formula (IH) and formula (IJ), n is 1. In other compounds of formula (IH) and formula (IJ), n is 2.

In other suitable compounds of the invention R¹ is H, R² is OH and n is 1 and the compound is of formula (IK) or a salt of formula (IL)

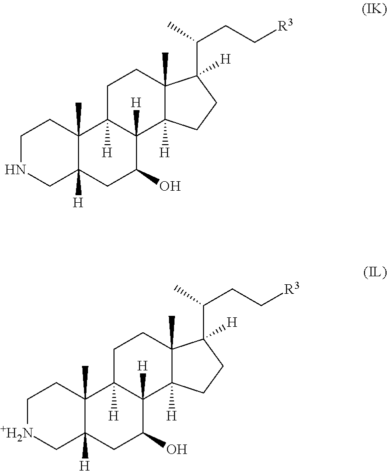

where R³ is as defined above for formula (I) or formula (IZ).

Particularly suitable compounds of the invention include:
tert-Butyl N-(benzyloxycarbony)-3-aza-7β-hydroxy-5β-cholan-24-oate (25a);
tert-Butyl 3-aza-7β-hydroxy-5β-cholan-24-oate (27a);
3-Aza-7β-hydroxy-5β-cholan-24-oic acid (28a);
tert-Butyl N-methyl-3-aza-7β-hydroxy-5β-cholan-24-oate (29a);
N-Methyl-3-aza-7β-hydroxy-5β-cholan-24-oic acid (31a and 33);
tert-Butyl N-ethyl-3-aza-7β-hydroxy-5β-cholan-24-oate (30);
N-Ethyl-3-aza-7β-hydroxy-5β-cholan-24-oic acid (32);
tert-Butyl N-glycolyl-3-aza-7β-hydroxy-5β-cholan-24-oate (34a);
N-Glycolyl-3-aza-7β-hydroxy-5β-cholan-24-oic acid (40a);
N-[(2S)-2-amino-3-methylbutanoyl]-3-aza-7β-hydroxy-5β-cholan-24-oic acid (41a);
N-[(2S,3S)-2-Amino-3-methylpentanoyl]-3-aza-7β-hydroxy-5β-cholan-24-oic acid (42a);
N-[(2S)-2-Amino-4-methylpentanoyl]-3-aza-7β-hydroxy-5β-cholan-24-oic acid (43a);
N-[(2S)-2-Amino-3-(1H-indol-3-yl)propanoyl]-3-aza-7β-hydroxy-5β-cholan-24-oic acid (44a);
N-[(2S)-2-Amino-3-(4-hydroxyphenyl)propanoyl]-3-aza-7β-hydroxy-5β-cholan-24-oic acid (45a);
tert-Butyl N-(benzyloxycarbonyl)-3-aza-7β-hydroxy-25-homo-5β-cholan-25-oate (25b);
tert-Butyl 3-aza-7β-hydroxy-25-homo-5β-cholan-25-oate (27b);
3-Aza-7β-hydroxy-25-homo-5β-cholan-25-oic acid (28b);
tert-Butyl N-methyl-3-aza-7β-hydroxy-25-homo-5β-cholan-25-oate (29b);
N-Methyl-3-aza-7β-hydroxy-25-homo-5β-cholan-25-oic acid (31b);
tert-Butyl N-glycolyl-3-aza-7β-hydroxy-25-homo-5β-cholan-25-oate (34b);
N-Glycolyl-3-aza-7β-hydroxy-25-homo-5β-cholan-25-oic acid (40b);
N-[(2S)-2-Amino-3-methylbutanoyl]-3-aza-7β-hydroxy-25-homo-5β-cholan-25-oic acid (41b);
N-[(2S,3S)-2-Amino-3-methylpentanoyl]-3-aza-7β-hydroxy-25-homo-5β-cholan-25-oic acid (42b);
N-[(2S)-2-Amino-4-methylpentanoyl]-3-aza-7β-hydroxy-25-homo-5β-cholan-25-oic acid (43b, Example 25);
N-[(2S)-2-Amino-3-(1H-indol-3-yl)propanoyl]-3-aza-7β-hydroxy-25-homo-5β-cholan-25-oic acid (44b);
N-(benzyloxycarbony)-3-aza-7β-hydroxy-5β-cholan-24-oic acid (50a);
N-(3-Aza-7β-hydroxy-5β-cholan-24-amide)-ethylsulfonic acid (51a);
N-(3-Aza-7β-hydroxy-5β-cholan-24-amide)-acetic acid (52a);
N-(3-Methyl-aza-7β-hydroxy-5β-cholan-24-oyl)-(2S)-2-amino-3-[(2-methylpropan-2-yl)oxy]propanoic acid (53a);
N-(3-Methyl-aza-7β-hydroxy-5β-cholan-24-oyl)-(R)-3-amino-3-phenylpropanoic acid (54a);
N-(3-Methyl-aza-7β-hydroxy-5β-cholan-24-oyl)-1-amino-4-fluorobenzene (55a);
N-(3-Aza-7β-hydroxy-5β-cholan-24-oyl)-morpholine (56a);
N-(3-Aza-7β-hydroxy-5β-cholan-24-oyl)-(S)-2-amino-3-hydroxypropanoic acid (57a);
N-(3-Aza-7β-hydroxy-5β-cholan-24-oyl)-3-amino-2-fluoropropanoic acid (58a);
N-(Cyclohexyl)-N-(3-methyl-aza-7β-hydroxy-5β-cholan-24-oyl)-cyclohexanamine (59a);
N-(3-Aza-7β-hydroxy-5β-cholan-24-oyl)-4-aminobenzoic acid (60a);
N-(3-Aza-7β-hydroxy-5β-cholan-24-oyl)-(S)-2-amino-4-(methylthio)butanoic acid (61a);
N-(3-Aza-7β-hydroxy-5β-cholan-24-amide)-propanoic acid (62a);
N-(3-Aza-7β-hydroxy-5β-cholan-24-oyl)-(isopropyl-4-aminobenzoate) (63a);
S-(3-Aza-7β-hydroxy-5β-cholan-24-oyl)-phenylmethanethiol (64a);
N-(3-Aza-7β-hydroxy-5β-cholan-24-oyl)-(S)-2-amino-3-methylbutanoic acid (65a);
N-(3-Aza-7β-hydroxy-5β-cholan-24-oyl)-(2S,3S)-2-amino-3-methylpentanoic acid (66a);
N-Methyl-N-(3-aza-7β-hydroxy-5β-cholan-24-oyl)-glycine (67a);
N-(3-Aza-7β-hydroxy-5β-cholan-24-oyl)-(S)-2-aminopropanoic acid (68a);
N-(benzyloxycarbonyl)-3-aza-7β-hydroxy-24-homo-5β-cholan-25-oic acid (50b);
N-(3-Aza-7β-hydroxy-27-homo-5β-cholan-27-oyl)-(S)-2-aminobutanedioic acid (70b);
N-(3-Aza-7β-hydroxy-27-homo-5β-cholan-27-oyl)-(2S)-2-amino-3-[(2-methylpropan-2-yl)oxy]propanoic acid (71b);
N-(3-Aza-7β-hydroxy-27-homo-5β-cholan-27-amide)-ethylsulfonic acid (72b);
N-(3-Aza-7β-hydroxy-27-homo-5β-cholan-27-oyl)-(S)-2-amino-3-phenylpropanoic acid (73b);
N-(3-Aza-7β-hydroxy-27-homo-5β-cholan-27-oyl)-3-aminobutanoic acid (74b);
N-(3-Aza-7β-hydroxy-27-homo-5β-cholan-27-oyl)-(S)-2-amino-4-methylpentanoic acid (75b);
N-(3-Aza-7β-hydroxy-27-homo-5β-cholan-27-oyl)-(2S)-2,6-diaminohexanoic acid (76b);
N-(3-Aza-7β-hydroxy-27-homo-5β-cholan-27-oyl)-(2S)-2-amino-3-(4-hydroxyphenyl)propanoic acid (77b);
N-{(benzyloxycarbonyl)-3-aza-7β-hydroxy-5β-cholan-24-oyl}-2-oxa-6-azospiro{3,3}heptane
(78a);
N-{(benzyloxycarbonyl)-3-aza-7β-hydroxy-5β-cholan-24-oyl}-4-piperidone (79a);
N-{(benzyloxycarbonyl)-3-aza-7β-hydroxy-25-homo-5β-cholan-25-oyl}-3-aminotetrahydrofuran (80b);
N-{(3-aza-7β-hydroxy-5β-cholan-25-oyl}-isoindoline (81a);
N-{(3-aza-7β-hydroxy-25-homo-5β-cholan-25-oyl)}-3-aminotetrahydrofuran (82b); and
   salts and solvates thereof.

Some still more suitable compounds of the invention include:
3-Aza-7β-hydroxy-5β-cholan-24-oic acid (28a);
N-Ethyl-3-aza-7β-hydroxy-5β-cholan-24-oic acid (32);
3-Aza-7β-hydroxy-25-homo-5β-cholan-25-oic acid (28b);
N-Methyl-3-aza-7β-hydroxy-25-homo-5β-cholan-25-oic acid (31b);
N-Glycolyl-3-aza-7β-hydroxy-25-homo-5β-cholan-25-oic acid (40b);
N-[(2S)-2-Amino-3-methylbutanoyl]-3-aza-7β-hydroxy-25-homo-5β-cholan-25-oic acid (41b);
   and
   salts and solvates thereof.

Other still more suitable compounds of the invention include:
N-(3-Aza-7β-hydroxy-5β-cholan-24-amide)-acetic acid (52a);
N-(3-Methyl-aza-7β-hydroxy-5β-cholan-24-oyl)-(R)-3-amino-3-phenylpropanoic acid (54a);
N-(3-Methyl-aza-7β-hydroxy-5β-cholan-24-oyl)-1-amino-4-fluorobenzene (55a);
N-(3-Aza-7β-hydroxy-5β-cholan-24-oyl)-(S)-2-amino-3-hydroxypropanoic acid (57a);
N-(3-Aza-7β-hydroxy-5β-cholan-24-amide)-propanoic acid (62a);
S-(3-Aza-7β-hydroxy-5β-cholan-24-oyl)-phenylmethanethiol (64a);
N-(3-Aza-7β-hydroxy-5β-cholan-24-oyl)-(S)-2-amino-3-methylbutanoic acid (65a);
N-(3-Aza-7β-hydroxy-5β-cholan-24-oyl)-(2S,3S)-2-amino-3-methylpentanoic acid (66a);
N-Methyl-N-(3-aza-7β-hydroxy-5β-cholan-24-oyl)-glycine (67a);
N-(3-Aza-7β-hydroxy-27-homo-5β-cholan-27-oyl)-(S)-2-amino-4-methylpentanoic acid (75b);
N-(3-Aza-7β-hydroxy-27-homo-5β-cholan-27-oyl)-(2S)-2-amino-3-(4-hydroxyphenyl)propanoic acid (77b);
N-{(benzyloxycarbonyl)-3-aza-7β-hydroxy-5β-cholan-24-oyl}-2-oxa-6-azospiro{3,3}heptane (78a);
N-{(benzyloxycarbonyl)-3-aza-7β-hydroxy-5β-cholan-24-oyl}-4-piperidone (79a);
N-{(benzyloxycarbonyl)-3-aza-7β-hydroxy-25-homo-5β-cholan-25-oyl}-3-aminotetrahydrofuran (80b); and
   salts and solvates thereof.

In some embodiments of the invention, the compound is not
N-(benzyloxycarbonyl)-3-aza-7β-hydroxy-24-homo-5β-cholan-25-oic acid (50b);
N-(3-Aza-7β-hydroxy-27-homo-5β-cholan-27-oyl)-(S)-2-amino-3-phenylpropanoic acid (73b);
N-(3-Aza-7β-hydroxy-27-homo-5β-cholan-27-oyl)-3-aminobutanoic acid (74b); or
   a salt or solvate of any of these.

### Preparation of Compounds of the Invention

Compounds of formula (I) in which R¹ is C(O)OR⁵ and R³ is C(O)OR¹⁶ may be prepared from compounds of formula (II):

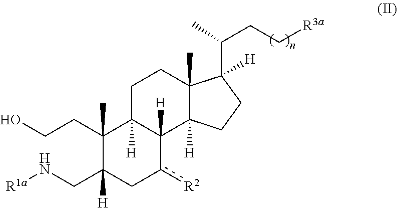

wherein R² and n are as defined for formula (I), R^{1a} is C(O)OR⁵ and R^{3a} is C(O)OR¹⁶, wherein R⁵ and R¹⁶ are as defined for formula (I);
by reaction with methane sulfonyl chloride in an organic solvent such as pyridine at reduced temperature, for example about −5° to 5° C.

Compounds of formula (II) are new and form a further aspect of the invention.

Compounds of formula (II) may be prepared respectively from compounds of formula (III):

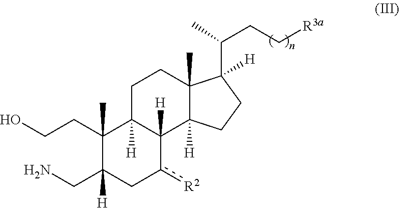

wherein R² and n are as defined for formula (I), and R^{3a} is as defined for formula (II); by reaction with a compound of formula (IV):

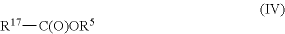

wherein R¹⁷ is halo, especially Cl or Br.

Suitably, the reaction is carried out under mildly basic conditions, for example in the presence of aqueous sodium carbonate. The solvent may also comprise an organic solvent such as dichloromethane.

Compounds of formula (III) may be prepared by oxidation of compounds of formula (V):

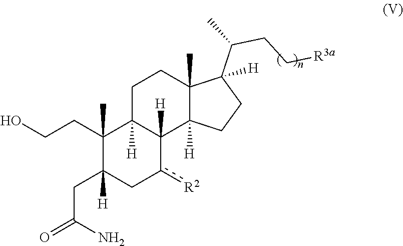

wherein R² and n are as defined for formula (I), and R^{3a} is as defined for formula (II).

The oxidation may be carried out using an oxidising agent such as (diacetoxyiodo)benzene, in which case, the reaction is suitably conducted at room temperature, for example at about 15° to 25° C. and in a polar organic solvent, for example a mixture of acetonitrile and water.

Compounds of formula (V) may be prepared from lactones of formula (VI):

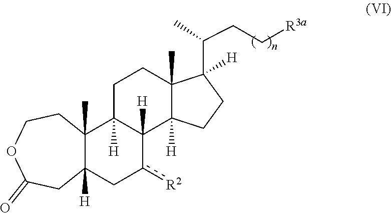

wherein R² and n are as defined for formula (I), and R^{3a} is as defined for formula (II);
by reaction with ammonia in an alcoholic solvent such as methanol.

The reaction may be carried out at elevated temperature, for example about 80° to 100° C., suitably in a sealed tube.

Compounds of formula (VI) may be prepared by oxidation of a compound of formula (VII):

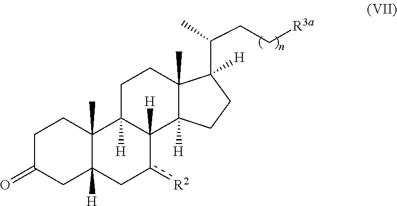

wherein R² and n are as defined for formula (I), and R^{3a} is as defined for formula (II).

Suitable oxidising agents include meta-chloroperbenzoic acid (mCPBA) and the reaction is suitably conducted at room temperature, for example at about 15° to 25° C. and in an organic solvent such as dichloromethane.

A compound of formula (VII) may be prepared by oxidation of a compound of formula (VIII):

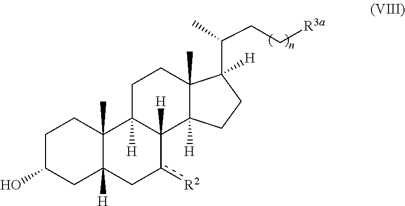

wherein R² and n are as defined for formula (I), and R^{3a} is as defined for formula (II).

Suitable oxidising agents for this reaction include (diacetoxyiodo)benzene together with a catalytic amount of 2,2,6,6-tetramethylpiperidine 1-oxyl radical (TEMPO) or a TEMPO derivative. The reaction may be conducted in dry conditions in an organic solvent such as dichloromethane.

A compound of formula (VIII) in which n is 1 may be prepared from a compound of formula (IX):

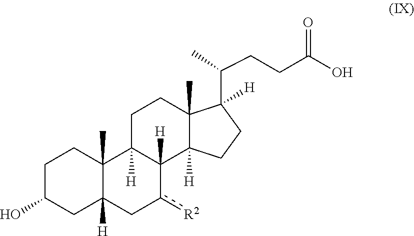

wherein R² is as defined for formula (I):
by reaction with trifluoroacetic acid followed by reaction of the product with an alcohol of formula (X):

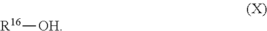

wherein R¹⁶ is as defined for formula (I).

Compounds of formulae (IX) and compounds of formula (X) are readily available or may be prepared by methods known to those of skill in the art. The compounds of formula (IX) in which [symbol] is a single bond and R² is OH are ursodeoxycholic acid and chenodeoxycholic acid. The compound of formula (IX) in which [symbol] is a double bond and R² is O is 7-keto lithocholic acid (7-KLCA).

Compounds of formula (VIII) in which n is 2 may be prepared from compounds of formula (IX) according to the following reaction scheme.

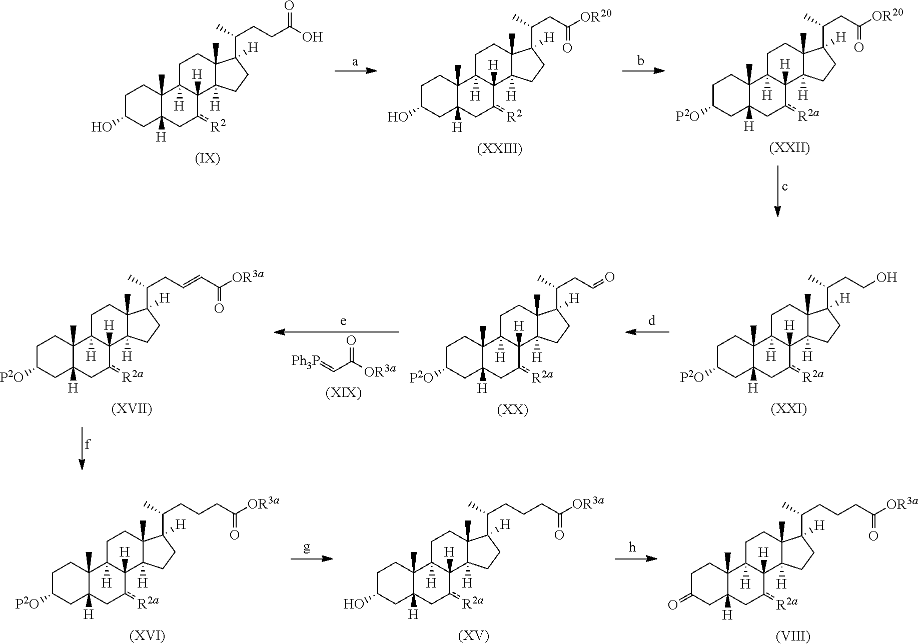

Step a: this step may be carried out according to the procedure described in D'Amore et al, 2014. The product is a compound of formula (XXIII) in which R² is as defined for formula (I) and R²⁰ is C₁₋₆ alkyl, for example methyl.
Step b: in this step, the compound of formula (XXIII) is reacted with a compound of formula:

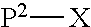

where P² is a protecting group, for example a silyl protecting group such as t-butyl dimethyl silyl chloride in the presence of a base such as imidazole. The reaction may be carried out at room temperature. The product is a protected compound of formula (XXII) in which R²⁰ is as defined for formula (XXIII), P² is as defined above, and R^{2a} is O when [symbol] is a double bond and R² is OP² when [symbol] is a single bond.
Step c: in this step, the compound of formula (XXII) is reduced to give an alcohol of formula (XXI), wherein P² and R^{2a} are as defined for formula (XXII). Suitable reducing agents include lithium borohydride and the reaction is suitably carried out in an organic solvent such as tetrahydrofuran at reduced temperature, typically from about −5° to 5° C., for example 0° C.
Step d: the compound of formula (XXI) is oxidised using a suitable oxidising agent such as oxalyl chloride to give a product of formula (XX) in which P² and R^{2a} are as defined for formula (XXII). The reaction may be carried out at a temperature of about −78° C.
Step e: the compound of formula (XX) is reacted with a compound of formula (XIX), wherein R^{3a} is as defined for formula (II). The reaction may be carried out in a polar organic solvent such as dichloromethane and at a temperature of about 15° to 25° C., typically at room temperature. The product is a compound of formula (XVII) in which R^{3a} is as defined for formula (II) and P² and R^{2a} are as defined for formula (XXII).
Step f: the compound of formula (XVII) is reduced to give a compound of formula (XVI) in which R^{3a} is as defined for formula (II) and P² and R^{2a} are as defined for formula (XXII). Suitably the reduction is carried out by hydrogenation over a suitable catalyst, for example palladium on carbon. Suitable reaction solvents include organic solvents such as ethyl acetate and the reaction may be carried out at a temperature of about 15° to 25° C., typically at room temperature.
Step g: the compound of formula (XVI) is deprotected to give a compound of formula (XV) in which R^{3a} is as defined for formula (II). Deprotection may be achieved by treatment with an acid, for example aqueous hydrochloric acid.
Step h: the compound of formula (XV) may be converted to a compound of formula (VIII) by oxidation. Suitable oxidising agents for this reaction include (diacetoxyiodo)benzene together with a catalytic amount of TEMPO or a TEMPO derivative. The reaction may be conducted in dry conditions in an organic solvent such as dichloromethane. The reaction may be conducted at a temperature of about 15° to 25° C., typically at room temperature.

Compounds of formula (I) may be converted to other compounds of formula (I). For example, compounds of formula (I) in which R¹ is H may be prepared from compounds of formula (I) in which R¹ is C(O)OR⁵, wherein R⁵ is as defined above for formula (IZ); by hydrogenation over a palladium catalyst. Suitably, the hydrogenation is carried out in an alcoholic solvent such as methanol.

In the compounds of formula (I), C(O)OR⁵ is suitably an amine protecting group and forms a carbamate with the nitrogen atom to which it is linked. R⁵ is still more suitably C₁₋₆ alkyl optionally substituted with a 6- to 14-membered aryl or 5- to 14-membered aryl group. For example, R⁵ may be t-butyl, benzyl or fluorenylmethyl.

Specific compounds of formula (I) in which R¹ is C(O)OR⁵ include:
tert-Butyl N-(benzyloxycarbony)-3-aza-7β-hydroxy-5β-cholan-24-oate (25a);
tert-Butyl N-(benzyloxycarbonyl)-3-aza-7β-hydroxy-25-homo-5β-cholan-25-oate (25b)
N-(benzyloxycarbonyl)-3-aza-7β-hydroxy-5β-cholan-24-oic acid (50a);
N-(benzyloxycarbonyl)-3-aza-7β-hydroxy-24-homo-5β-cholan-25-oic acid (50b);
N-{(benzyloxycarbonyl)-3-aza-7β-hydroxy-5β-cholan-24-oyl}-2-oxa-6-azospiro{3,3}heptane (78a);
N-{(benzyloxycarbonyl)-3-aza-7β-hydroxy-5β-cholan-24-oyl}-4-piperidone (79a);
N-{(benzyloxycarbonyl)-3-aza-7β-hydroxy-25-homo-5β-cholan-25-oy}-3-aminotetrahydrofuran (80b); and
   salts and solvates thereof.

A compound of formula (I) in which R¹ is H and R³ is C(O)OR¹⁶ may be converted to a compound of formula (I) in which R¹ is C₁₋₆ alkyl and R³ is C(O)OR¹⁶ by reaction with a compound of formula (XX):

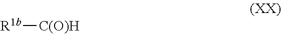

wherein R^{1b} is C₁₋₅ alkyl;
followed by hydrogenation over a palladium/carbon catalyst.

The reaction may be carried out in an aqueous solvent.

A compound of formula (I) in which R¹ is H and R³ is C(O)OR¹⁶ may be converted to a compound of formula (I) in which R¹ is C₂₋₆ alkenyl or C₂₋₆ alkynyl and R³ is C(O)OR¹⁶ by the reaction of the starting material with a compound of formula (XVIII):

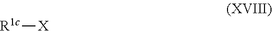

wherein R^{1c} is C₂₋₆ alkenyl or C₂₋₆ alkynyl and X is a leaving group such as halo, for example chloro or bromo;
in the presence of a base.

For compounds of formula (I) in which R² is OH, the process may further comprise an initial step of reacting the starting compound of formula (I) with a suitable protecting group and a final step of deprotecting the product of the reaction with the compound of formula (XVIII.

If required, the product in which R¹ is H and R³ is C(O)OR¹⁶ may be converted to a compound of formula (I) with a different R³ group by one of the methods described below.

A compound of formula (I) in which R³ is C(O)OR¹⁶ may be hydrolysed with an acid or a base to give a compound of formula (I) in which R³ is C(O)OH. In particular, hydrolysis may be carried out using an acid such as trifluoroacetic acid. Suitably, the reaction is carried out at reduced temperature, for example at about −5° to 5° C. under anhydrous conditions and in a polar organic solvent such as dichloromethane.

A compound of formula (I) in which R¹ is H may be converted to a salt of formula (I) in which the nitrogen atom to which R¹ is attached is quaternised by treatment with an acid such as hydrochloric acid or trifluoroacetic acid. This reaction is particularly useful for converting compounds of formula (I) in which R¹ is H and R³ is C(O)OH or C(O)OR¹⁶ to quaternary ammonium salts of formula (I) in which R³ is C(O)OH.

A compound of formula (I) in which R¹ is C(O)R⁴ and R³ is C(O)OR¹⁶ may be prepared from a compound of formula (I) in which R¹ is H and R³ is C(O)OR¹⁶ by reaction with a compound of formula (XXI):

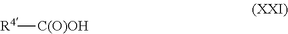

wherein R^{4′} is C₁₋₆ alkyl optionally substituted with one or more substituents selected from OR^{14a}, N(R^{14a})(R^{14b}), NH₃′, C(O)N(R^{14a})(R^{14b}), SR¹⁴ a protected OH group, a protected NH₂ group, a protected C(O)NH₂ group, a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl);
under basic conditions, for example using N,N-diisopropylethylamine, and in the presence of a coupling agent; and
where necessary, removal of a protecting group to give a group R⁴ comprising an OH, NH₂ or C(O)NH₂ group.

Suitable coupling reagents include known peptide coupling agents such as O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(7-azabenzotriazol-1-yl)-N,N,N′,N′-tetramethyluronium tetrafluoroborate (TATU), (benzotriazol-1-yloxy)tris(dimethylamino)phosphonium hexafluorophosphate (BOP), (benzotriazol-1-yloxy)tripyrrolidinophosphonium hexafluorophosphate (PyBOP) carbodiimides such as 1-ethyl-3-(3-dimethylaminopropyl)carbodiimide (EDCI) and triazoles such as 1-hydroxy-7-azabenzotriazole (HOAt) or hydroxybenzotriazole (HOBt); and chloroformates such as isobutyl chloroformate.

The product may be converted to a compound of formula (I) in which R³ is C(O)OH by reaction with an acid as described above. In this case, the treatment with trifluoroacetic acid will also lead to the removal of protecting groups from protected OH and NH₂ groups in R⁴.

The product may be a compound of formula (IP2):

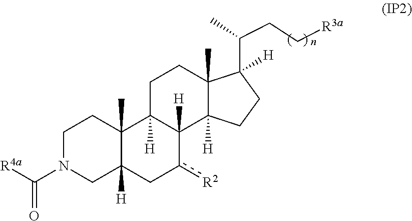

wherein R² and n are as defined for formula (I), R³ is as defined for formula (II) and R^{4a} is C₁₋₆ alkyl substituted with a protected NH₂ group, a protected OH group or a protected C(O)NH₂ group.

The protected NH₂ group, a protected OH group or a protected C(O)NH₂ groups are chosen such that treatment with the acid results in the removal of the protecting group to give an acid addition salt of a compound of formula (I) in which R² and n are as defined above, R³ is C(O)OH and R¹ is C(O)R⁴, where R⁴ is C₁₋₆ alkyl substituted with NH₂ or OH or C(O)NH₂. Suitable protected NH₂ groups include acid labile carbamates, for example t-butyl carbamate. Suitable protected OH groups include ethers, for example monomethyl ether or tetrahydropyran Suitable protected C(O)NH₂ groups include alkyl substituted amides, for example C(O)NH-tBu

Compounds of formula (IP2) are new and form a further aspect of the invention in which there is provided a compound of formula (IP2):

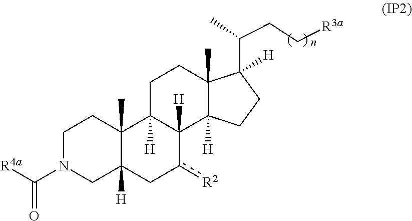

wherein R² and n are as defined for formula (I), R³ is as defined for formula (II) and R^{4a} is C₁₋₆ alkyl substituted with a protected NH₂ group, a protected OH group or a protected C(O)NH₂ group.

Specific compounds of formula (IP2) include:
tert-Butyl N-{(2S)-2-[(tert-butoxycarbonyl)amino]-3-methylbutanoyl}-3-aza-7β-hydroxy-5β-cholan-24-oate (35a);
tert-Butyl N-{(2S,3S)-2-[(tert-butoxycarbonyl)amino]-3-methylpentanoyl}-3-aza-7β-hydroxy-5β-cholan-24-oate (36a);
tert-Butyl N-{(2S)-2-[(tert-butoxycarbonyl)amino]-4-methylpentanoyl}-3-aza-7β-hydroxy-5β-cholan-24-oate (37a);
tert-Butyl N-{(2S)-2-[(tert-butoxycarbonyl)amino]-3-[1H-indol-3-yl]propanoyl}-3-aza-7β-hydroxy-5β-cholan-24-oate (38a);
tert-Butyl N-{(2S)-2-[(tert-butoxycarbonyl)amino]-3-[4-hydroxyphenyl]propanoyl}-3-aza-7β-hydroxy-5β-cholan-24-oate (39a);
tert-Butyl N-{(2S)-2-[(tert-butoxycarbonyl)amino]-3-methylbutanoyl}-3-aza-7β-hydroxy-25-homo-5β-cholan-25-oate (35b);
tert-Butyl N-{(2S,3S)-2-[(tert-butoxycarbonyl)amino]-3-methylpentanoyl}-3-aza-7β-hydroxy-25-homo-5β-cholan-25-oate (36b);
tert-Butyl N-{(2S)-2-[(tert-butoxycarbonyl)amino]-4-methylpentanoyl}-3-aza-7β-hydroxy-25-homo-5β-cholan-25-oate (37b);
tert-Butyl N-{(2S)-2-[(tert-butoxycarbonyl)amino]-3-[1H-indol-3-yl]propanoyl}-3-aza-7β-hydroxy-25-homo-5β-cholan-25-oate (38b); and
   salts and solvates thereof.

Compounds of formula (I) in which R³ is C(O)N(R⁶)—X¹—R⁷ may be prepared from compounds of formula (I) in which R³ is C(O)OH by reaction with a compound of formula (XXV):

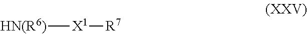

wherein R⁶, R⁷ and X¹ are as defined for formula (I);
in the presence of a coupling reagent and under basic conditions, for example in the presence of an amine such as diisopropylethylamine (DIPEA) or triethylamine (TEA) and in an organic solvent such as DMF. Suitable coupling agents are as described above.

Similarly, compounds of formula (I) in which R³ is C(O)N(R⁸)(R⁹) may be prepared by reacting a compound formula (I) in which R³ is C(O)OH with a compound of formula (XXVI):

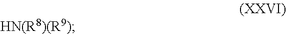

where R⁸ and R⁹ are as defined for formula (I);
under basic conditions and in the presence of a coupling agent. Suitable coupling agents are as described above.

Compounds of formula (I) in which R³ is C(O)S—R¹⁰ may be prepared by reacting a compound formula (I) in which R³ is C(O)OH with a compound of formula (XXVII):

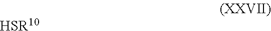

where R¹⁰ is as defined for formula (I).

Compounds of formulae (XXV), (XXVI) and (XXVII) are known and are readily available or may be synthesised by methods known to those of skill in the art.

### Therapeutic Methods

Surprisingly, it has been shown that the compounds of the invention are able to restore mitochondrial function and can cross the blood brain barrier. They are therefore of use in the treatment or prevention of neurodegenerative disorders including Parkinson's disease, mild cognitive impairment, dementia (including Alzheimer's disease, vascular dementia, dementia with Lewy bodies and frontotemporal dementia (FTD)), Huntington's disease, amyotrophic lateral sclerosis (motor neurone disease), progressive supranuclear palsy and Wilson's disease.

In the discussion below, references to compounds of formula (I) and formula (IZ) for use in medicine, the use of compounds of formula (I) and formula (IZ) in the preparation of a medicament, methods of treatment employing compounds of formula (I) and pharmaceutical compositions comprising compounds of formula (I) and formula (IZ) apply equally to the pharmaceutically acceptable salts and solvates of compounds of formula (I) and formula (IZ).

Where a compound of formula (IZ) is used in medical applications:
R¹ is suitably not C(O)OR⁵; and
when R¹ is C(O)R⁴, R⁵ is suitably not substituted with a protected OH group, a protected NH₂ group, a protected C(O)NH₂ group; and
when R³ is C(O)N(R⁶)—X¹—R⁷, X¹ is suitably not substituted with a protected OH group, a protected NH₂ group or a protected C(O)NH₂ group.

The compounds of formula (I) and formula (IZ) are also useful in treating or preventing conditions in which modulating mitochondrial function is advantageous, particularly neurodegenerative disorders such as Parkinson's disease, mild cognitive impairment, dementia (including Alzheimer's disease, vascular dementia, dementia with Lewy bodies and FTD), Huntington's disease, amyotrophic lateral sclerosis (motor neurone disease), progressive supranuclear palsy and Wilson's disease.

The compounds of formula (I) and formula (IZ) are also of use in treating or preventing acute radiation syndrome, for example treating a human or animal patient who has been exposed to radiation or who is likely to exposed to radiation, for example as in the case of an astronaut undertaking space travel.

The compounds of the invention are also of use in the treatment or prevention of myalgic encephalomyelitis (ME, chronic fatigue syndrome) and post viral syndrome, including chronic symptoms arising from infection with SARS-CoV2 (long COVID).

In a further aspect of the invention, there is provided a compound of formula (I) or formula (IZ) for use in medicine.

There is also provided:
A compound of formula (I) or formula (IZ) for use in the treatment of a neurodegenerative disorder;
A compound of formula (I) or formula (IZ) for use in the prevention of a neurodegenerative disorder
A compound of formula (I) or formula (IZ) for use in the treatment of acute radiation syndrome;
A compound of formula (I) or formula (IZ) for use in the prevention of acute radiation syndrome;
A compound of formula (I) or formula (IZ) for use in the treatment of myalgic encephalomyelitis (ME, chronic fatigue syndrome) or post viral syndrome, including chronic symptoms arising from infection with SARS-CoV2 (long COVID);
A compound of formula (I) or formula (IZ) for use in the prevention of myalgic encephalomyelitis (ME, chronic fatigue syndrome) or post viral syndrome, including chronic symptoms arising from infection with SARS-CoV2 (long COVID).

The invention also provides:
The use of a compound of formula (I) or formula (IZ) in the preparation of an agent for the treatment of a neurodegenerative disorder;
The use of a compound of formula (I) or formula (IZ) in the preparation of an agent for the prevention of a neurodegenerative disorder;
The use of a compound of formula (I) or formula (IZ) in the preparation of an agent for the treatment of acute radiation syndrome;
The use of a compound of formula (I) or formula (IZ) in the preparation of an agent for the prevention of acute radiation syndrome;
The use of a compound of formula (I) or formula (IZ) in the preparation of an agent for the treatment of myalgic encephalomyelitis (ME, chronic fatigue syndrome) or post viral syndrome, including chronic symptoms arising from infection with SARS-CoV2 (long COVID);
The use of a compound of formula (I) or formula (IZ) in the preparation of an agent for the prevention of myalgic encephalomyelitis (ME, chronic fatigue syndrome) or post viral syndrome, including chronic symptoms arising from infection with SARS-CoV2 (long COVID).

The invention further provides:
A method for the treatment of a neurodegenerative disorder, the method comprising administering to a patient in need of such treatment an effective amount of a compound of formula (I) or formula (IZ);
A method for the prevention of a neurodegenerative disorder, the method comprising administering to a patient in need of such treatment an effective amount of a compound of formula (I) or formula (IZ);
A method for the treatment of acute radiation syndrome, the method comprising administering to a patient in need of such treatment an effective amount of a compound of formula (I) or formula (IZ);
A method for the prevention of acute radiation syndrome, the method comprising administering to a patient in need of such treatment an effective amount of a compound of formula (I) or formula (IZ);
A method for the treatment of myalgic encephalomyelitis (ME, chronic fatigue syndrome) or post viral syndrome, including chronic symptoms arising from infection with SARS-CoV2 (long COVID), the method comprising administering to a patient in need of such treatment an effective amount of a compound of formula (I) or formula (IZ);
A method for the prevention of myalgic encephalomyelitis (ME, chronic fatigue syndrome) or post viral syndrome, including chronic symptoms arising from infection with SARS-CoV2 (long COVID), the method comprising administering to a patient in need of such treatment an effective amount of a compound of formula (I) or formula (IZ).

Examples of neurodegenerative disorders include Parkinson's disease, mild cognitive impairment, dementia (including Alzheimer's disease, vascular dementia, dementia with Lewy bodies and FTD), Huntington's disease, amyotrophic lateral sclerosis (motor neurone disease), progressive supranuclear palsy and Wilson's disease. Disorders which are particularly suitable for treatment with the compounds of the present invention include Parkinson's disease, mild cognitive impairment, dementia (including Alzheimer's disease, vascular dementia, dementia with Lewy bodies and FTD), Huntington's disease and amyotrophic lateral sclerosis and especially Parkinson's disease, mild cognitive impairment and dementia (including Alzheimer's disease, vascular dementia, dementia with Lewy bodies and FTD).

### Pharmaceutical Compositions

The compounds of formula (I) and formula (IZ) will generally be administered as part of a pharmaceutical composition.

Therefore, in a further aspect of the invention, there is provided a pharmaceutical composition comprising a compound of formula (I) or formula (IZ) or a pharmaceutically acceptable salt or solvate thereof and a pharmaceutically acceptable excipient or carrier.

The composition may be formulated for administration by any route, for example parenteral, including intravenous, intramuscular, subcutaneous or intradermal; or oral, rectal, nasal, topical (including transdermal, eye drops, topical administration to the lung, buccal and sublingual) or vaginal administration.

More suitably, the composition is formulated for parenteral administration or for oral administration, topical administration to the skin (transdermal administration) or topical administration to the lung (by inhalation).

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of formula (I) in conjunction or association with a pharmaceutically acceptable excipient or carrier.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

In some cases, the compositions may be formulated for delayed, slow or controlled release of the compound of formula (I) or formula (IZ).

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, compounds of formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC). Suitable CFC propellants include trichloromonofluoromethane (propellant 11), dichlorotetrafluoromethane (propellant 114), and dichlorodifluoromethane (propellant 12). Suitable HFC propellants include tetrafluoroethane (HFC-134a) and heptafluoropropane (HFC-227). The propellant typically comprises 40%-99.5% e.g. 40%-90% by weight of the total inhalation composition. The formulation may comprise excipients including co-solvents (e.g. ethanol) and surfactants (e.g. lecithin, sorbitan trioleate and the like). Other possible excipients include polyethylene glycol, polyvinylpyrrolidone, glycerine and the like. Aerosol formulations are packaged in canisters and a suitable dose is delivered by means of a metering valve (e.g. as supplied by Bespak, Valois or 3M or alternatively by Aptar, Coster or Vari).

Topical administration to the lung may also be achieved by use of a non-pressurised formulation such as an aqueous solution or suspension. These may be administered by means of a nebuliser e.g. one that can be hand-held and portable or for home or hospital use (ie non-portable). The formulation may comprise excipients such as water, buffers, tonicity adjusting agents, pH adjusting agents, surfactants and co-solvents. Suspension liquid and aerosol formulations (whether pressurised or unpressurised) will typically contain the compound of the invention in finely divided form, for example with a D₅₀ of 0.5-10 μm e.g. around 1-5 μm. Particle size distributions may be represented using D₁₀, D₅₀ and D₉₀ values. The D₅₀ median value of particle size distributions is defined as the particle size in microns that divides the distribution in half. The measurement derived from laser diffraction is more accurately described as a volume distribution, and consequently the D₅₀ value obtained using this procedure is more meaningfully referred to as a Dv₅₀ value (median for a volume distribution). As used herein Dv values refer to particle size distributions measured using laser diffraction. Similarly, D₁₀ and D₉₀ values, used in the context of laser diffraction, are taken to mean Dv₁₀ and Dv₉₀ values and refer to the particle size whereby 10% of the distribution lies below the D₁₀ value, and 90% of the distribution lies below the D₉₀ value, respectively.

Topical administration to the lung may also be achieved by use of a dry-powder formulation. A dry powder formulation will contain the compound of the disclosure in finely divided form, typically with a mass mean diameter (MMAD) of 1-10 μm or a D₅₀ of 0.5-10 μm e.g. around 1-μm. Powders of the compound of the invention in finely divided form may be prepared by a micronization process or similar size reduction process. Micronization may be performed using a jet mill such as those manufactured by Hosokawa Alpine. The resultant particle size distribution may be measured using laser diffraction (e.g. with a Malvern Mastersizer 2000S instrument). The formulation will typically contain a topically acceptable diluent such as lactose, glucose or mannitol (preferably lactose), usually of comparatively large particle size e.g. a mass mean diameter (MMAD) of 50 μm or more, e.g. 100 μm or more or a D₅₀ of 40-150 μm. As used herein, the term “lactose” refers to a lactose-containing component, including α-lactose monohydrate, β-lactose monohydrate, α-lactose anhydrous, β-lactose anhydrous and amorphous lactose. Lactose components may be processed by micronization, sieving, milling, compression, agglomeration or spray drying. Commercially available forms of lactose in various forms are also encompassed, for example Lactohale® (inhalation grade lactose; DFE Pharma), InhaLac® 70 (sieved lactose for dry powder inhaler; Meggle), Pharmatose® (DFE Pharma) and Respitose® (sieved inhalation grade lactose; DFE Pharma) products. In one embodiment, the lactose component is selected from the group consisting of α-lactose monohydrate, α-lactose anhydrous and amorphous lactose. Preferably, the lactose is α-lactose monohydrate.

Dry powder formulations may also contain other excipients. Thus in one embodiment a dry powder formulation according the present disclosure comprises magnesium or calcium stearate. Such formulations may have superior chemical and/or physical stability especially when such formulations also contain lactose.

A dry powder formulation is typically delivered using a dry powder inhaler (DPI) δevice. Example dry powder delivery systems include SPINHALER®, DISKHALER®, TURBOHALER®, DISKUS®, SKYEHALER®, ACCUHALER® and CLICKHALER®. Further examples of dry powder delivery systems include ECLIPSE, NEXT, ROTAHALER, HANDIHALER, AEROLISER, CYCLOHALER, BREEZHALER/NEOHALER, MONODOSE, FLOWCAPS, TWINCAPS, X-CAPS, TURBOSPIN, ELPENHALER, MIATHALER, TWISTHALER, NOVOLIZER, PRESSAIR, ELLIPTA, ORIEL dry powder inhaler, MICRODOSE, PULVINAL, EASYHALER, ULTRAHALER, TAIFUN, PULMOJET, OMNIHALER, GYROHALER, TAPER, CONIX, XCELOVAIR and PROHALER.

In one embodiment a compound of formula (I) is provided as a micronized dry powder formulation, for example comprising lactose of a suitable grade.

Thus, as an aspect of the invention there is provided a pharmaceutical composition comprising a compound of formula (I) or formula (IZ) in particulate form in combination with particulate lactose, said composition optionally comprising magnesium stearate.

In one embodiment a compound of formula (I) or formula (IZ) is provided as a micronized dry powder formulation, comprising lactose of a suitable grade and magnesium stearate, filled into a device such as DISKUS. Suitably, such a device is a multidose device, for example the formulation is filled into blisters for use in a multi-unit dose device such as DISKUS.

In another embodiment a compound of formula (I) or formula (IZ) is provided as a micronized dry powder formulation, for example comprising lactose of a suitable grade, filled into hard shell capsules for use in a single dose device such as AEROLISER.

In another embodiment a compound of formula (I) or formula (IZ) is provided as a micronized dry powder formulation, comprising lactose of a suitable grade and magnesium stearate, filled into hard shell capsules for use in a single dose device such as AEROLISER.

In another embodiment a compound of formula (I) or formula (IZ) is provided as a fine powder for use in an inhalation dosage form wherein the powder is in fine particles with a D₅₀ of 0.5-μm e.g. around 1-5 μm, that have been produced by a size reduction process other than jet mill micronisation e.g. spray drying, spray freezing, microfluidisation, high pressure homogenisation, super critical fluid crystallisation, ultrasonic crystallisation or combinations of these methods thereof, or other suitable particle formation methods known in the art that are used to produce fine particles with an aerodynamic particle size of 0.5-10 μm. The resultant particle size distribution may be measured using laser diffraction (e.g. with a Malvern Mastersizer 2000S instrument). The particles may either comprise the compound alone or in combination with suitable other excipients that may aid the processing. The resultant fine particles may form the final formulation for delivery to humans or may optionally be further formulated with other suitable excipients to facilitate delivery in an acceptable dosage form. The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Parenteral formulations will generally be sterile.

The medical practitioner, or other skilled person, will be able to determine a suitable dosage for the compound of formula (I) or formula (IZ), and hence the amount of the compound of the invention that should be included in any particular pharmaceutical formulation (whether in unit dosage form or otherwise).

Compounds of formula (I) or formula (IZ) may be used in combination with one or more other active agents which are useful in the treatment or prophylaxis of neurodegenerative disorders, acute radiation syndrome or myalgic encephalomyelitis (ME, chronic fatigue syndrome) or post viral syndrome, including chronic symptoms arising from infection with SARS-CoV2 (long COVID).

Therefore, in a further aspect of the invention, there is provided a pharmaceutical composition as described above further comprising an additional active agent useful in the treatment or prophylaxis of neurodegenerative disorders, acute radiation syndrome or myalgic encephalomyelitis (ME, chronic fatigue syndrome) or post viral syndrome, including chronic symptoms arising from infection with SARS-CoV2 (long COVID).

There is also provided a product comprising a compound of formula (I) or formula (IZ) and an additional active agent useful in the treatment or prevention of a neurodegenerative disorder as a combined preparation for simultaneous, sequential or separate use in the treatment or prevention of a neurodegenerative disorder as described above.

There is also provided a product comprising a compound of formula (I) or formula (IZ) and an additional active agent useful in the treatment or prevention of acute radiation syndrome as a combined preparation for simultaneous, sequential or separate use in the treatment or prevention of acute radiation syndrome.

There is also provided a product comprising a compound of formula (I) or formula (IZ) and an additional active agent useful in the treatment or prevention of myalgic encephalomyelitis (ME, chronic fatigue syndrome) or post viral syndrome, including chronic symptoms arising from infection with SARS-CoV2 (long COVID), as a combined preparation for simultaneous, sequential or separate use in the treatment or prevention of ME or long COVID.

### FIGURES AND EXAMPLES

The invention will now be further described with reference to the following examples and to the drawings in which:

FIG. 1 shows data obtained from measuring mitochondrial respiration using the Seahorse Mito Stress test. Data are presented from 3 control fibroblast lines and 3 sporadic Parkinson's disease patient fibroblast lines, each treated both with vehicle and with Compound 28b (Example 18). Each test was repeated at 3 independent passages. The bars represent mean values from the 3 passages and error bars represent the standard deviation. FIG. 1A shows spare respiratory capacity, FIG. 1B shows basal mitochondrial respiration and FIG. 1C shows ATP linked respiration.

FIG. 2 shows mitochondrial respiratory chain complex I activity in WT C57B6 mouse brain hemispheres (3 per group) in an untreated control group (Con un in the figure) and in a group treated with Compound 28b (Example 18) at 1, 4, 8, 12 and 24 hours after dosing. Complex I activity is elevated after dosing with Compound 28b 1 hour post dosing, remained elevated at the same level at 8 hours post dosing. The levels remained elevated, although to a lesser extent by 24 hours post dosing.

### GENERAL EXPERIMENTAL PROCEDURES

Proton (¹H) and carbon (¹³C) NMR-spectra were recorded on Bruker Avance (III)-500 spectrometer. Chemical shifts are reported in ppm relative to Me₄Si (TMS, d 0), or residual solvent peaks as an internal standard set to d 7.26 and 77.00 (CDCl₃), or d 3.34 and 49.05 (CDOD), or d 2.50 and 39.43 (d₆-DMSO). NMR data is reported as follows: chemical shift in ppm, multiplicity (ap=apparent, s=singlet, d=doublet, t=triplet, q=quartet, sp=septet, br=broad, dd=doublet of doublets, td=triplet of doublets, dt=doublet of triplets, m=multiplet), coupling constant in Hz, integration.

Electrospray ionization (ESI) mass spectrometry (MS) experiments were performed on a QTOF Premier mass spectrometer (Micromass, UK) under normal conditions. Sodium formate solution was used as calibrant for high resolution mass spectra (HRMS) measurements. All reactions were monitored by thin layer chromatography (TLC) using 0.2 mm silica gel (Merck Kieselgel 60 F₂₅₄) precoated aluminium plates, using UV light, ammonium molybdate, ninhydrin or potassium permanganate staining solution to visualize. Flash column chromatography was performed on Davisil® silica gel (60, particle size 0.040-0.063 mm), or using Reveleris® silica or C-18 reversed phase flash cartridges on a Grace Reveleris® automated flash system with continuous gradient facility. Solvents for reactions and chromatography were analytical grade and were used as supplied unless otherwise stated. Chiral and achiral high performance liquid chromatography (HPLC) analyses were performed on an Agilent 1100 (Quaternary pump) HPLC system with a refractive index detector, employing columns as indicated. Data was processed with Agilent Cerity System software.

### Example 1

### tert-Butyl N-(benzyloxycarbony)-3-aza-7β-hydroxy-5β-cholan-24-oate (25a)

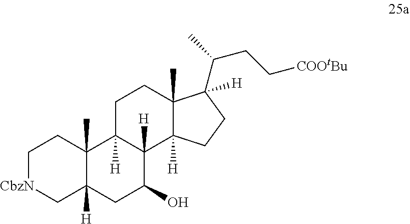

### A. tert-Butyl 3α,7β-dihydroxy-5β-cholan-24-oate (12)

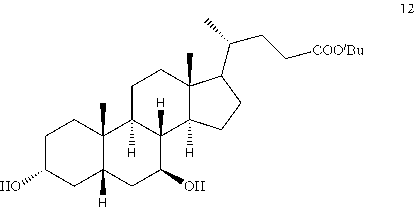

To a solution of ursodeoxycholic acid (10.2 g, 26.0 mmol) in dry THF (225 mL) was added dropwise trifluoroacetic anhydride (30.0 mL, 216 mmol) at 0° C. After complete addition the ice bath was removed and the reaction was stirred for 1.5 h. Subsequently, tert-butanol (63.8 mL) was introduced portionwise at room temperature and the reaction was stirred at room temperature overnight. Then concentrated aqueous ammonia (53 mL) was added at room temperature and again the reaction was left stirring overnight. Saturated bicarbonate solution was added and the aqueous phase was extracted with ethyl acetate (3×). The organic fractions were combined, washed with brine, dried over MgSO₄ and concentrated. The crude product was purified by automated flash column chromatography (silica gel, ethyl acetate/petroleum ether 10-100%) to yield the product 12 quantitatively as a colourless foam.

¹H NMR (500 MHz, CDCl₃) δ 3.64-3.54 (m, 2H), 2.26 (ddd, 15.2, 9.9, 5.3 Hz, 1H), 2.13 (ddd, 15.3, 9.5, 6.8 Hz, 1H), 2.00 (dt, 12.5, 3.1 Hz, 1H), 1.96-1.86 (m, 1H), 1.84-1.72 (m, 4H), 1.71-1.64 (m, 2H), 1.64-1.54 (m, 2H), 1.53-1.36 (m, 6H; 1.44, s, 9H), 1.36-1.20 (m, 5H), 1.15 (td, 12.9, 3.8 Hz, 1H), 1.10-0.98 (m, 2H), 0.95 (s, 3H), 0.92 (d, 6.6 Hz, 3H), 0.68 (s, 3H); HRMS (ESI) m/z calcd for C₂₈H₄₈O₄Na⁺ 471.3445, found 471.3440.

### B. tert-Butyl 7β-Hydroxy-3-oxo-5β-cholan-24-oate (13)

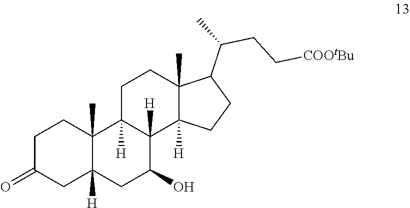

To a solution of 12, the product of step A (26.0 mmol) in dry dichloromethane (180 mL) was added (diacetoxyiodo)benzene (BAIB; 10.3 g, 32.0 mmol) and a catalytic amount of TEMPO (624 mg, 3.99 mmol). After being stirred overnight the reaction was quenched with saturated Na₂S₂O₃ solution and diluted with water. The organic layer was separated and the aqueous layer was extracted another three times with dichloromethane. The organic phases were combined, washed with brine, dried over MgSO₄ and concentrated. The residue was purified by automated flash column chromatography (silica gel, ethyl acetate/petroleum ether 2-100%) to give 11.4 g (98%) of the product 13 as a light yellowish foam.

¹H NMR (500 MHz, CDCl₃) δ 3.61 (ddd, 11.3, 8.7, 5.2 Hz, 1H), 2.52 (dd, 15.0, 13.9 Hz, 1H), 2.32-2.22 (m, 2H), 2.22-2.09 (m, 3H), 2.09-2.00 (overlapping signals: 2.06, dt, 13.1, 3.2 Hz, 1H; 2.02, ddd, ˜14.3, 5.3, 3.3 Hz, 1H), 1.98-1.88 (m, 2H), 1.87-1.73 (m, 3H), 1.62 (ddd, 13.5, 5.1, 2.4 Hz, 1H), 1.58-1.34 (m, 9H; 1.44, s, 9H), 1.34-1.24 (m, 2H), 1.21 (td, 12.6, 4.7 Hz, 1H), 1.09 (dt, 9.7, 9.6 Hz, 1H), 1.05 (s, 3H), 0.94 (d, 6.6 Hz, 3H), 0.72 (s, 3H); HRMS (ESI) m/z calcd for C₂₈H₄₆O₄Na⁺ 469.3288, found 469.3292.

### C. tert-Butyl 7β-Hydroxy-4-oxa-3-oxo-4α-homo-5-cholan-24-oate (Side Product 14a) and tert-butyl 7β-Hydroxy-3-oxa-4-oxo-4α-homo-5-cholan-24-oate (Required Product 15a)

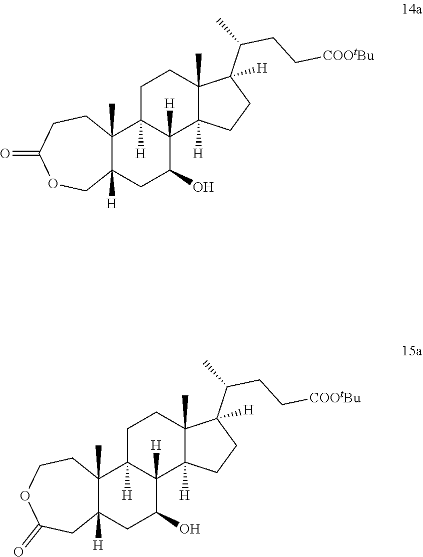

To a solution of 13, the product of step B (11.3 g, 25.3 mmol) in dichloromethane (300 mL)) was added meta-chloroperbenzoic acid (mCPBA, 57-86%; 13.8 g, ˜56.0 mmol) in one portion at room temperature and the resulting reaction mixture was stirred overnight. The reaction was quenched with saturated Na₂S₂O₃ solution which was followed by addition of saturated bicarbonate solution. The organic layer was separated and the aqueous phase was extracted with ethyl acetate (3×). The combined organic fractions were washed with brine, dried over MgSO₄ and concentrated. The crude product was purified by automated flash column chromatography (silica gel, ethyl acetate/petroleum ether 2-80%) to yield 10.6 g (91%) of 14a and 15a as a 1:1 mixture of isomers (colourless foam).

¹H NMR (500 MHz, CDCl₃, mixture of isomers) δ 4.48 (dd, 13.1, 9.6 Hz, 1H), 4.17 (dd, 13.2, 10.2 Hz, 1H), 4.05 (ddd, 13.3, 6.5, 1.1 Hz, 1H), 3.98 (ap d, 12.9 Hz, 1H), 3.48-3.40 (m, 1H), 3.33-3.25 (m, 1H), 3.03 (ap t, 12.9 Hz, 1H), 2.63 (ap dd, 14.4, 13.1 Hz, 1H), 2.42-2.35 (m, 2H), 2.26 (ddd, 15.3, 9.9, 5.4 Hz, 2H), 2.13 (ddd, 15.4, 9.4, 6.8 Hz, 2H), 2.09-2.01 (m, 3H), 1.98-1.85 (m, 7H), 1.85-1.68 (m, 6H), 1.58-1.31 (m, 18H; 1.44, s, 18H), 1.31-1.15 (m, 6H), 1.12-1.03 (overlapping signals: m, 2H; 1.05, s, 3H; 1.04, s, 3H), 0.93 (d, 6.6 Hz, 3H), 0.92 (d, 6.6 Hz, 3H), 0.70 (s, 6H);

HRMS (ESI) m/z calcd for C₂₈H₄₆O₅Na⁺ (mixture of isomers) 485.3237, found 485.3250.

### D. tert-Butyl 4,7β-dihydroxy-3,4-seco-5β-cholan-24-oate-3-amide (Side Product 16a) and tert-butyl 2,7β-dihydroxy-2,3-seco-5β-cholan-24-oate-4-amide (Required Product 17a)

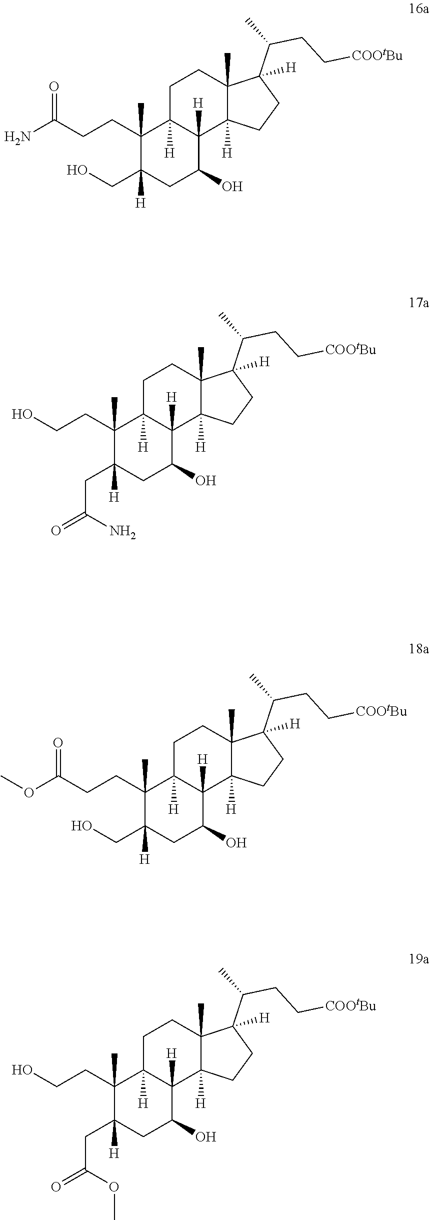

A solution of a mixture of lactones 14a and 15a from step C (10.6 g, 22.9 mmol) in dry 7 N ammonia in methanol (ca. 165 mL) was heated in a 200 mL sealed tube with Teflon screw cap at 90° C. overnight (the sealed tube was filled to 4/5 of its volume and a blast shield was added). The reaction was concentrated and the residue purified by automated flash column chromatography (silica gel, methanol/ethyl acetate 0-25%) to give 8.95 g (81%) of a mixture of amides (16a and 17a) as a colourless oil. The product ratio (16a:17a) was determined to be 1:1.2 by HPLC analysis (Phenomenex Luna C18(2) 5 μm 250×4.6 mm; Phenomenex Security Guard C18 4×3 mm; mobile phase: 45:55:0.05 water/acetonitrile/trifluoroacetic acid; flow rate: 1 mL/min; sample solvent: methanol; column temperature: 35° C.; injection volume: 25 μL; detection: refractive index). In addition 1.55 g (14%) of the corresponding esters 18a and 19a was isolated as colourless foam. The ratio of 18a:19a was determined to be 1:6.8 by HPLC analysis (Phenomenex Luna C18(2) 5 μm 250×4.6 mm; Phenomenex Security Guard C18 4×3 mm; mobile phase: 30:70:0.1 water/acetonitrile/trifluoroacetic acid; flow rate: 1 mL/min; sample solvent: methanol; column temperature: 35° C.; injection volume: 25 μL; detection: refractive index). An analytical sample of each of the four compounds was recovered for spectroscopic characterization.

tert-Butyl 4,7β-dihydroxy-3,4-seco-5β-cholan-24-oate-3-amide (16a).

¹H NMR (500 MHz, CDCl₃) δ 6.44 (s_{br}, 1H, NH₂), 6.06 (s_{br}, 1H, NH₂), 3.76-3.68 (m, 1H), 3.56-3.49 (m, 1H), 3.49-3.41 (m, 1H), 2.36-2.20 (overlapping signals: m, 1H; 2.25, ddd, 15.3, 9.8, 5.5 Hz, 1H), 2.17-2.07 (m, 2H), 1.99-1.94 (m, 2H), 1.93-1.71 (m, 4H), 1.71-1.63 (m, 1H), 1.62-1.52 (m, 2H), 1.51-1.36 (m, 4H; 1.44, s, 9H), 1.36-1.21 (m, 3H), 1.21-1.02 (m, 3H), 1.02-0.94 (overlapping signals: m, 1H; 0.97, s, 3H), 0.91 (d, 6.5 Hz, 3H), 0.68 (s, 3H); H,H—COSY (500 MHz, CDCl₃) δ 3.72 g 1.67, 3.53 g 1.67;

HRMS (ESI) m/z calcd for C₂₈H₄₉NO₅Na⁺ 502.3503, found 502.3511.

tert-Butyl 2,7β-dihydroxy-2,3-seco-5β-cholan-24-oate-3-amide (17a). ¹H NMR (500 MHz, CDCl₃) δ 6.75 (s_{br}, 1H, NH₂), 6.35 (s_{br}, 1H, NH₂), 3.82-3.72 (m, 1H), 3.72-3.63 (m, 1H), 3.63-3.54 (m, 1H), 3.49-2.39 (m, 1H), 2.25 (ddd, 15.3, 9.5, 5.2 Hz, 1H), 2.21-2.08 (m, 2H), 2.06-1.96 (m, 2H), 1.92-1.59 (m, 6H), 1.58-1.35 (m, 5H; 1.44, s, 9H), 1.35-1.11 (m, 5H), 1.11-1.01 (overlapping signals: m, 1H; 1.05, s, 3H), 1.00-0.87 (overlapping signals: m, 1H; 0.92, d, 6.4 Hz, 3H), 0.68 (s, 3H); H,H—COSY (500 MHz, CDCl₃) δ 3.75 g 1.71/1.39, 3.67 g 1.71/1.39;

HRMS (ESI) m/z calcd for C₂₈H₄₉NO₅Na⁺ 502.3503, found 502.3510. tert-Butyl 4,7β-dihydroxy-3,4-seco-5β-cholan-24-oate-3-methyl ester (18a). ¹H NMR (500 MHz, CDCl₃) δ 3.70 (dd, 10.8, 4.6 Hz, 1H), 3.67 (s, 3H), 3.60-3.50 (m, 2H), 2.37 (ddd, 15.5, 12.1, 4.5 Hz, 1H), 2.31-2.20 (m, 2H), 2.16-2.01 (overlapping signals: 2.13, ddd, 15.4, 9.5, 6.8 Hz, 1H; m, 3H), 1.98 (dt, 12.7, 3.2 Hz, 1H), 1.94-1.85 (m, 1H), 1.84-1.71 (m, 3H), 1.68-1.63 (m, 1H), 1.61-1.53 (m, 2H), 1.52-1.35 (m, 4H; 1.44, s, 9H), 1.35-1.22 (m, 3H), 1.19 (ddd, 10.6, 12.3, 7.3 Hz, 1H),), 1.15-0.95 (overlapping signals: 1.11, td, 13.0, 3.9 Hz, 1H; m, 2H; 0.99, s, 3H), 0.91 (d, 6.6 Hz, 3H), 0.68 (s, 3H); H,H—COSY (500 MHz, CDCl₃) δ 3.70 g 1.66, 3.56 g 1.66;

HRMS (ESI) m/z calcd for C₂₉H₅₀O₆Na⁺ 517.3500, found 517.3512.

tert-Butyl 2,7β-dihydroxy-2,3-seco-5β-cholan-24-oate-3-methyl ester (19a). ¹H NMR (500 MHz, CDCl₃) δ 3.77-3.68 (m, 2H), 3.68 (s, 3H), 3.58 (dt, 7.4, 9.4 Hz, 1H), 2.44 (dd, 14.8, 3.4 Hz, 1H), 2.33 (dd, 14.8, 11.4 Hz, 1H), 2.25 (ddd, 15.2, 9.9, 5.3 Hz, 1H), 2.12 (ddd, 15.3, 9.5, 6.8 Hz, 1H), 2.07-1.97 (m, 2H), 1.95-1.86 (m, 1H), 1.82-1.64 (m, 5H), 1.58-1.34 (m, 7H; 1.44, s, 9H), 1.34-1.24 (m, 3H), 1.21 (ddd, 10.6, 12.3, 7.3 Hz, 1H), 1.13 (td, 13.1, 4.0 Hz, 1H), 1.10-1.02 (overlapping signals: m, 1H; 1.08, s, 3H), 1.00-0.93 (m, 1H), 0.92 (d, 6.6 Hz, 3H), 0.68 (s, 3H); H,H—COSY (500 MHz, CDCl₃) δ 3.72 (2H) g 1.74/1.39;

HRMS (ESI) m/z calcd for C₂₉H₅₀O₆Na⁺ 517.3500, found 517.3510.

### E. tert-Butyl 2-amino-4,7β-dihydroxy-3-nor-3,4-seco-5β-cholan-24-oate (Side Product, 20a) and tert-butyl 4-amino-2,7β-dihydroxy-3-nor-3,4-seco-5β-cholan-24-oate (Required Product, 21a)

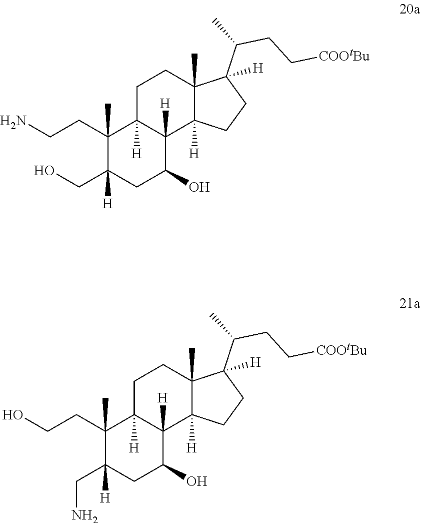

To a solution of the mixture of amide products from step D 16a and 17a (11.7 a. 24.4 mmol) in a 3:1 mixture of acetonitrile and water (590 mL/197 mL; ca. 30 mL of ethyl acetate was added to help solubilize the starting material) was added (diacetoxyiodo)benzene (BAIB; 9.09 g, 28.2 mmol) in one portion at room temperature. After being stirred overnight the reaction mixture was transferred into a 2 L flask and carefully concentrated to dryness. The residue was re-dissolved in methanol, passed along A 26-resin and concentrated. The crude product was then purified by automated flash column chromatography [silica gel, ethyl acetate/(methanol:28% aqueous ammonia solution 9:1) 0-80%] to yield 9.28 g (84%) of a mixture of the product amines 20a and 21a as a colourless semi solid.

¹H NMR (500 MHz, CD₃OD; mixture of isomers) δ 3.69 (dd, 10.9, 3.9 Hz, 1H), 3.68-3.56 (m, 2H), 3.52 (ap t, 10.4 Hz, 1H), 3.49-3.38 (m, 2H), 2.79-2.67 (m, 2H), 2.66-2.58 (m, 2H), 2.25 (ddd, 14.9, 9.2, 5.5 Hz, 2H), 2.14 (ddd, 15.1, 8.5, 7.7 Hz, 2H), 2.11-2.05 (m, 1H), 2.04-1.98 (m, 2H), 1.95-1.73 (overlapping signals: 1.92, ddd, 13.8, 4.9, 2.9 Hz, 1H; m, 6H), 1.68 (ddd, 13.5, 10.3, 5.3 Hz, 1H), 1.63-1.53 (m, 6H), 1.53-1.34 (m, 8H; 1.44, s, 18H), 1.35-1.11 (m, 11H), 1.10-0.96 (overlapping signals: m, 4H; 1.07, s, 3H; 1.05, s, 3H), 0.94 (d, 6.6 Hz, 6H), 0.72 (s, 6H); HRMS (ESI) m/z calcd for C₂₇H₄₉NO₄H⁺452.3734, found 452.3730.

### F. tert-Butyl N-(benzyloxycarbonyl)-2-amino-4,7β-dihydroxy-3-nor-3,4-seco-5β-cholan-24-oate (Side Product 22a) and tert-butyl N-(benzyloxycarbonyl)-4-amino-2,7β-dihydroxy-3-nor-3,4-seco-5β-cholan-24-oate (Required Product 23a)

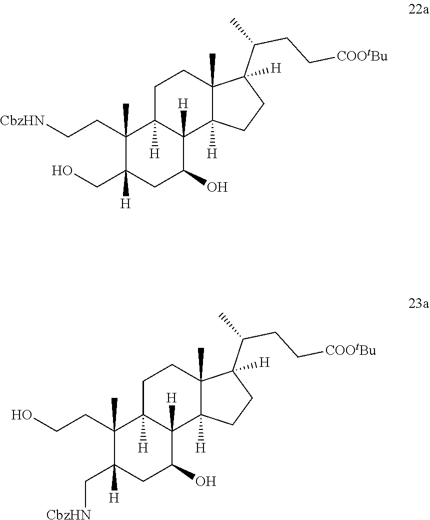

To a solution of a mixture of 20a and 21a, the products of Step E (10.9 g, 24.1 mmol) in a 1:1 mixture of dichloromethane (160 mL) and water (160 mL) was added sodium carbonate (13.1 g, 124 mmol) and the mixture was cooled to 0° C. Then, benzyl chloroformate (CbzCl 95%; 4.00 mL, 28.0 mmol) was introduced dropwise and the reaction was stirred for 40 min at 0° C. After complete reaction (TLC analysis) 28% aqueous ammonia solution (50 mL) was added and the resulting mixture was diluted with water. The mixture was extracted with ethyl acetate (3×) and the combined organic phases were washed with brine, dried over MgSO₄ and concentrated. The crude product mixture was purified by automated column chromatography (silica gel, ethyl acetate/petroleum ether 2-100%) to afford 5.95 g (42%) of the products 22a and 5.11 g (36%) of 23a, both as colourless foams.

### tert-Butyl N-(benzyloxycarbonyl)-2-amino-4,7β-dihydroxy-3-nor-3,4-seco-5β-cholan-24-oate (22a)

¹H NMR (500 MHz, CDCl₃) δ 7.40-7.29 (m, 5H), 5.12-5.05 (m, 2H), 4.88-4.79 (m, 1H, NH), 3.92-3.84 (m, 1H), 3.61-3.52 (m, 1H), 3.51-3.44 (m, 1H), 3.44-3.34 (m, 1H), 3.14-3.04 (m, 1H), 2.37-2.20 (overlapping signals: s_{br}, 1H, OH; 2.25, ddd, 15.3, 9.9, 5.3 Hz, 1H), 2.12 (ddd, 15.4, 9.3, 6.7 Hz, 1H), 2.06-1.94 (overlapping signals: m, 1H; 1.97, dt, 12.6, 3.1 Hz, 1H), 1.94-1.84 (m, 1H), 1.82-1.71 (m, 3H), 1.68-1.54 (m, 2H), 1.54-1.35 (m, 6H; 1.44, s, 9H), 1.35-1.21 (m, 3H), 1.17 (ddd, 12.1, 10.7, 7.4 Hz, 1H), 1.13-0.94 (overlapping signals: 1.10, td, 13.1, 3.8 Hz, 1H; m, 2H; 1.03, s, 3H), 0.91 (d, 6.6 Hz, 3H), 0.68 (s, 3H); H,H—COSY (500 MHz, CDCl₃) δ 3.88 g 1.75, 3.57 g 1.75;

tert-Butyl N-(Benzyloxycarbonyl)-4-amino-2,7β-dihydroxy-3-nor-3,4-seco-5β-cholan-24-oate (23a). ¹H NMR (500 MHz, CDCl₃) δ 7.41-7.29 (m, 5H), 5.09 (s, 2H), 4.96-4.90 (m, 1H, NH), 3.91-3.83 (m, 1H), 3.75-3.66 (m, 1H), 3.63-3.53 (m, 1H), 3.46-3.38 (m, 1H), 3.11 (ddd, 13.4, 11.5, 6.7 Hz, 1H), 2.25 (ddd, 15.3, 9.9, 5.3 Hz, 1H), 2.12 (ddd, 15.3, 9.5, 6.8 Hz, 1H), 2.02-1.95 (m, 1H), 1.96-1.86 (m, 1H), 1.83 (ddd, 13.7, 4.9, 2.7 Hz, 1H), 1.80-1.71 (m, 3H), 1.70-1.62 (m, 2H), 1.62-1.30 (m, 8H; 1.44, s, 9H), 1.30-1.17 (m, 3H), 1.17-1.08 (m, 1H), 1.08-0.97 (overlapping signals: m, 2H; 1.04, s, 3H), 0.91, d, 6.6 Hz, 3H), 0.68 (s, 3H); H,H—COSY (500 MHz, CDCl₃) δ 3.87 g 1.76, 1.49, 3.71 g 1.76, 1.49;

HRMS (ESI) m/z calcd for C₃₅H₅₅NO₆Na⁺ 608.3922, found 608.3931.

### G. tert-Butyl N-(benzyloxycarbony)-3-aza-7β-hydroxy-5β-cholan-25-oate (25a)

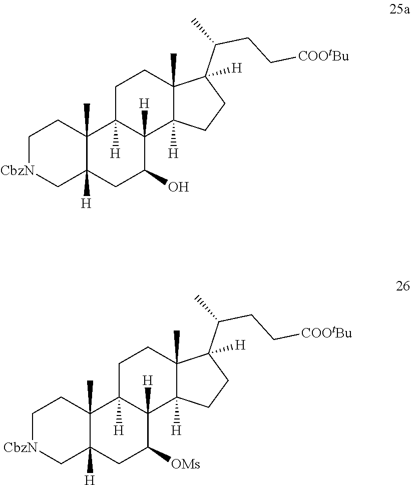

To a solution of 23a (2.83 g, 4.83 mmol) in dry pyridine (90 mL) was added dropwise methanesulfonyl chloride (0.45 mL, 5.81 mmol) at 0° C. The resulting reaction mixture was stirred for 2 h at this temperature and once only traces of staring material were detectable (TLC analysis) saturated bicarbonate solution (5.8 mL) was introduced. The reaction was allowed to warm to room temperature and subsequently stirred at 74° C. overnight. The solvent was evaporated and the residue was taken up in water. The mixture was extracted with ethyl acetate (3×), the combined organic fractions were washed with brine, dried over MgSO₄, filtered through a plug of celite and concentrated. The crude product was purified by automated column chromatography (silica gel, ethyl acetate/petroleum ether 0-60%) to yield 2.02 g (74%) of 25a as a colourless foam. In addition, a sample of the over-mesylated by-product 26 (24.2 mg) was recovered.

tert-Butyl N-(benzyloxycarbonyl)-3-aza-7β-hydroxy-5β-cholan-25-oate (25a). ¹H NMR (500 MHz, CDCl₃) δ 7.39-7.28 (m, 5H), 5.18-5.05 (m, 2H), 3.98-3.75 (m, 2H), 3.57-3.45 (m, 1H), 3.08-2.91 (m, 1H), 2.89-2.73 (m, 1H), 2.25 (ddd, 15.3, 9.9, 5.3 Hz, 1H), 2.12 (ddd, 15.4, 9.5, 6.8 Hz, 1H), 2.01 (dt, 12.8, 3.1 Hz, 1H), 1.96-1.86 (m, 1H), 1.84-1.70 (m, 4H), 1.70-1.58 (m, 2H), 1.55-1.39 (m, 6H; 1.44, s, 9H), 1.39-1.11 (overlapping signals: m, 5H; 1.15, td, 12.9, 4.1 Hz, 1H), 1.07 (dt, 9.7, 9.7 Hz, 1H), 0.99 (s, 3H), 0.92 (d, 6.6 Hz, 3H), 0.69 (s, 3H);

HRMS (ESI) m/z calcd for C₃₅H₅₃NO₅Na⁺ 590.3816, found 590.3815.

tert-Butyl N-(benzyloxycarbonyl)-3-aza-7β-mesyloxy-5β-cholan-25-oate (26). ¹H NMR (500 MHz, CDCl₃) δ 7.39-7.28 (m, 5H), 5.12 (s_{br}, 2H), 4.65-4.57 (m, 1H), 4.00-3.80 (m, 2H), 3.04-2.94 (overlapping signals: m, 1H; 2.98, s, 3H), 2.87-2.71 (m, 1H), 2.24 (ddd, 15.2, 9.7, 5.4 Hz, 1H), 2.16-2.05 (m, 3H), 2.01 (dt, 12.8, 3.2 Hz, 1H), 1.93-1.81 (m, 2H), 1.81-1.71 (m, 3H), 1.71-1.62 (m, 1H), 1.62-1.55 (m, 1H), 1.52-1.20 (m, 8H; 1.44, s, 9H), 1.16 (td, 12.9, 3.8 Hz, 1H), 1.07 (dt, 9.5, 9.0 Hz, 1H), 1.01 (s, 3H), 0.91 (d, 6.5 Hz, 3H), 0.68 (s, 3H);

HRMS (ESI) m/z calcd for C₃₆H₅₅NO₇SNa⁺ 668.3591, found 668.3590.

### Example 2

### tert-Butyl 3-aza-7β-hydroxy-5β-cholan-24-oate (27a)

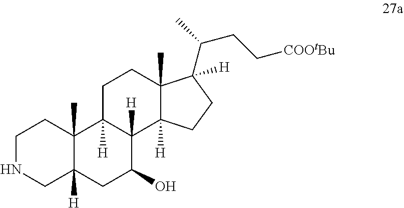

To a solution of 25a from Example 1 (1.47 g, 2.58 mmol) in methanol (30 mL) was added 10% palladium on charcoal (92.7 mg) and the atmosphere was exchanged for hydrogen. The resulting reaction mixture was stirred at room temperature overnight. After complete deprotection (TLC analysis) the reaction was filtered through celite and concentrated. The residual colourless oil was purified by automated column chromatography [silica gel, (methanol/28% aqueous ammonia solution 9:1)/ethyl acetate 0-60%] to give 1.04 g (93%) of 27a as a colourless foam.

¹H NMR (500 MHz, CDCl₃) δ 3.48 (ddd, 10.9, 9.0, 5.2 Hz, 1H), 3.00-2.87 (m, 3H), 2.76 (td, 13.1, 2.0 Hz, 1H), 2.26 (ddd, 15.3, 9.9, 5.3 Hz, 1H), 2.13 (ddd, 15.4, 9.4, 6.8 Hz, 1H), 2.01 (dt, 12.7, 3.2 Hz, 1H), 1.97-1.87 (m, 1H), 1.87-1.72 (m, 5H), 1.64-1.56 (m, 1H), 1.55-1.19 (m, 10H; 1.44, s, 9H), 1.15 (td, 12.9, 4.3 Hz, 1H), 1.08 (dt, 9.7, 9.7 Hz, 1H), 1.03 (s, 3H), 0.92 (d, 6.6 Hz, 3H), 0.68 (s, 3H);

HRMS (ESI) m/z calcd for C₂₇H₄₇NO₃H⁺ 434.3629, found 434.3627.

### Example 3

### 3-Aza-7β-hydroxy-5β-cholan-24-oic acid hydrochloride salt (28a)

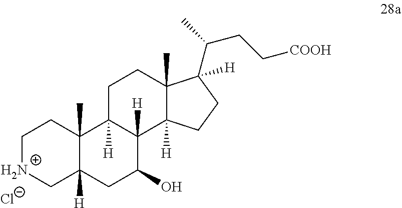

To a solution of the compound of Example 2 (27a) (153 mg, 0.353 mmol) in dry dichloromethane (4 mL) was added trifluoroacetic acid (TFA; 3 mL) at 0° C. After being stirred at this temperature for 2.5 h the reaction was concentrated and the residue co-evaporated with a 3 M aqueous hydrochloric acid solution (3×) followed by THF (1×). The crude product was dried under high vacuum and re-dissolved in THF (˜60 mL). The resulting mixture was concentrated to a small volume in an open 100 mL round bottomed flask. The precipitate was filtered off, washed with THF and dried under high vacuum. Residual THF was subsequently removed by co-evaporation with methanol. The sample was then dried under high vacuum at 80° C. to afford 110 mg (75%) of the product 28a as an amorphous colourless solid.

¹H NMR (500 MHz, CD₃OD) δ 3.42-3.34 (m, 1H), 3.24 (t, 12.8 Hz, 1H), 3.15-3.05 (m, 2H), 3.01 (td, 13.5, 2.3 Hz, 1H), 2.33 (ddd, 15.3, 9.8, 5.4 Hz, 1H), 2.20 (ddd, 15.6, 9.2, 6.8 Hz, 1H), 2.10-1.99 (m, 2H), 1.96-1.76 (m, 5H), 1.66-1.38 (m, 8H), 1.38-1.22 (m, 4H), 1.16-1.05 (overlapping signals: m, 1H; 1.09, s, 3H), 0.96 (d, 6.6 Hz, 3H), 0.73 (s, 3H);

¹³C NMR (125 MHz, CD₃OD) δ 178.17, 71.03, 56.92, 56.58, 45.02, 44.78, 44.35, 42.06, 41.16, 40.80, 39.98, 36.70, 35.17, 34.26, 33.55, 32.40, 32.09, 29.62, 27.91, 23.47, 22.54, 18.95, 12.62;

HRMS (ESI) m/z calcd for C₂₃H₃₉NO₃H⁺ 378.3003, found 378.3004.

### Example 4

### tert-Butyl N-methyl-3-aza-7β-hydroxy-5β-cholan-24-oate (29a)

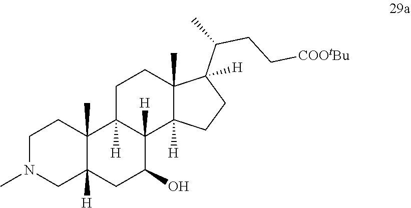

To a solution of 25a, the product of Example 1 (203 mg, 0.358 mmol) in methanol (10 mL) was added formalin (37% in water; 0.15 mL, 2.02 mmol) and the mixture was stirred at room temperature for 1 h. Subsequently, 10% palladium on charcoal was added (37.8 mg) and the atmosphere was exchanged for hydrogen. After being stirred overnight the reaction was filtered through a plug of celite and the filtrate was concentrated. The crude product was purified by automated column chromatography [silica gel, (methanol:28% aqueous ammonia solution 9:1)/ethyl acetate 0-20%] to give 161 mg (quant.) of the product 29a as a colourless oil.

¹H NMR (500 MHz, CDCl₃) δ 3.53 (ddd, 11.0, 9.1, 5.2 Hz, 1H), 2.58-2.48 (m, 2H), 2.29-2.09 (overlapping signals: m, 1H; 2.26, s, 3H; 2.19, ap t, 11.7 Hz, 1H; 2.13, ddd, 15.4, 9.5, 6.7 Hz, 1H), 2.03-1.96 (m, 2H), 1.95-1.86 (m, 1H), 1.85-1.72 (m, 5H), 1.63-1.56 (m, 1H), 1.54-1.21 (m, 10H; 1.44, s, 9H), 1.16 (td, 12.9, 3.8 Hz, 1H), 1.08 (dt, 9.6, 9.6 Hz, 1H), 0.99 (s, 3H), 0.92 (d, 6.5 Hz, 3H), 0.68 (s, 3H);

HRMS (ESI) m/z calcd for C₂₈H₄₉NO₃H⁺ 448.3785, found 448.3782.

### Example 5

### N-Methyl-3-aza-7β-hydroxy-5β-cholan-24-oic acid hydrotrifluoroacetate (31a)

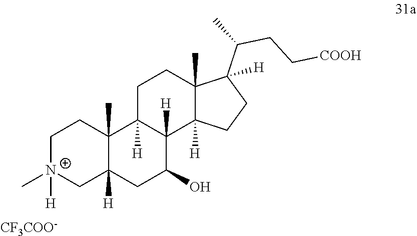

To a solution of 29a, the product of Example 4, (93.0 mg, 0.208 mmol) in dry dichloromethane (5 mL) was added trifluoroacetic acid (4 mL) at 0° C. and the resulting mixture was stirred for 2.5 h. Then the reaction was concentrated and the crude product was purified by automated column chromatography [C18 silica gel, acetonitrile/water (+0.5% trifluoroacetic acid) 2-40%]. The purified product was co-evaporated with dichloromethane (3-4×) and methanol (1×) at 60° C. to give 51.1 mg (51%) of 31a as a colourless foam.

¹H NMR (500 MHz, CD₃OD) δ 3.38 (ddd, 11.2, 9.8, 5.1, 1H), 3.30-3.18 (m, 3H), 3.05-2.97 (m, 1H), 2.86 (s, 3H), 2.33 (ddd, 15.4, 9.8, 5.4 Hz, 1H), 2.21 (ddd, 15.5, 9.3, 6.9 Hz, 1H), 2.10-2.02 (m, 2H), 1.96-1.77 (m, 5H), 1.66-1.55 (m, 2H), 1.53-1.37 (m, 6H), 1.37-1.23 (m, 4H), 1.16-1.06 (overlapping signals: m, 1H; 1.08, s, 3H); 0.96 (d, 6.6 Hz, 3H), 0.73 (s, 3H);

¹⁹F NMR (470 MHz, CD₃OD) δ −77.00;

¹³C NMR (125 MHz, CD₃OD) δ 178.13, 70.94, 56.79, 56.61, 55.41, 51.07, 44.78, 44.37, 43.75, 42.96, 41.10, 39.79, 36.69, 35.06, 34.42, 33.47, 32.39, 32.08, 29.61, 27.90, 23.05, 22.55, 18.95, 12.61;

HRMS (ESI) m/z calcd for C₂₄H₄₁NO₃H⁺ 392.3159, found 392.3169.

### Example 6

### N-Methyl-3-aza-7β-hydroxy-5β-cholan-24-oic acid hydrochloride (33)

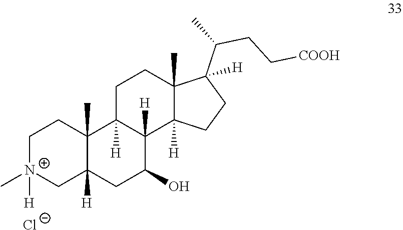

To a solution of 29a, the product of Example 4, (161 mg, 0.360 mmol) in dry dichloromethane (5 mL) was added trifluoroacetic acid (4 mL) at 0° C. After being stirred for 2.5 h the reaction was concentrated and the residue purified by automated column chromatography [C18 silica gel, acetonitrile/water (+0.5% trifluoroacetic acid) 2-40%]. The purified product was co-evaporated with dichloromethane (3-4×) and a solution of 3 M aqueous hydrochloric acid (3×). The resulting product was dried under high vacuum and in a desiccator over potassium carbonate to yield 112 mg (73%) of 33 as a colourless foam.

¹H NMR (500 MHz, CD₃OD) δ 3.38 (ddd, 11.3, 9.8, 5.2, 1H), 3.33-3.17 (m, 3H), 3.06-2.97 (m, 1H), 2.86 (s, 3H), 2.33 (ddd, 15.3, 9.8, 5.4 Hz, 1H), 2.20 (ddd, 15.5, 9.3, 6.9 Hz, 1H), 2.12-2.03 (m, 2H), 1.96-1.77 (m, 5H), 1.67-1.55 (m, 2H), 1.54-1.38 (m, 6H), 1.38-1.23 (m, 4H), 1.16-1.07 (overlapping signals: m, 1H; 1.09, s, 3H), 0.96 (d, 6.6 Hz, 3H), 0.74 (s, 3H);

¹³C NMR (125 MHz, CD₃OD) δ 178.10, 70.93, 56.76, 56.59, 55.43, 51.10, 44.78, 44.36, 43.81, 42.94, 41.10, 39.76, 36.69, 35.07, 34.43, 33.50, 32.39, 32.08, 29.63, 27.91, 23.04, 22.56, 18.96, 12.62;

HRMS (ESI) m/z calcd for C₂₄H₄₁NO₃H⁺ 392.3159, found 392.3174.

### Example 7

### tert-Butyl N-ethyl-3-aza-7β-hydroxy-5β-cholan-24-oate (30)

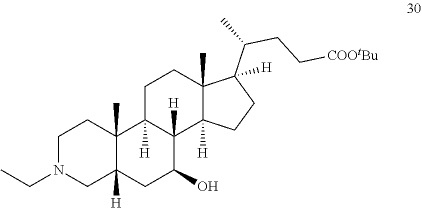

Using an analogous method to that of Example 4, the compound of Example 1 (25a) (111 mg, 0.196 mmol) was dissolved in methanol (6 mL), treated with acetaldehyde (0.06 mL, 1.07 mmol) and subsequently hydrogenated on 10% palladium on charcoal (22.4 mg) to afford 56.4 mg (63%) of the product 30 as a colourless foam.

¹H NMR (500 MHz, CDCl₃) δ 3.53 (ddd, 11.0, 9.3, 5.2, 1H), 2.69-2.58 (m, 2H), 2.40 (q, 7.2H, 2H), 2.25 (ddd, 15.3, 9.9, 5.3 Hz, 1H), 2.19-2.08 (m, 2H), 2.03-1.86 (m, 3H), 1.85-1.72 (m, 5H), 1.64-1.56 (m, 1H), 1.54-1.21 (m, 11H; 1.44, s, 9H), 1.15 (td, 12.9, 3.9 Hz, 1H), 1.12-1.04 (overlapping signals: m, 1H; 1.08, t, 7.2 Hz, 3H), 0.99 (s, 3H), 0.92 (d, 6.6 Hz, 3H), 0.68 (s, 3H);

HRMS (ESI) m/z calcd for C₂₉H₅₁NO₃H⁺ 462.3942, found 462.3942.

### Example 8

### N-Ethyl-3-aza-7β-hydroxy-5β-cholan-24-oic acid hydrotrifluoroacetate (32)

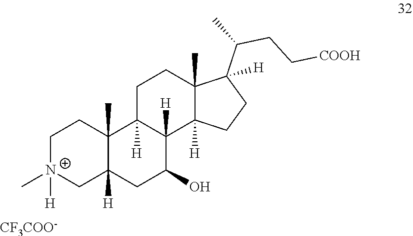

Using an analogous method to that of Example 5, the compound of Example 7 (30) (50.0 mg, 0.108 mmol) was dissolved in dry dichloromethane (5 mL) and reacted with trifluoroacetic acid (4 mL) at 0° C. for 2.5 h to give 26.4 mg (47%) of the product 32 as a colourless foam.

¹H NMR (500 MHz, CD₃OD) δ 3.39 (ddd, 11.3, 9.7, 5.2, 1H), 3.33-3.24 (m, 2H), 3.24-3.13 (overlapping signals: m, 1H; 3.17, q, 7.5 Hz, 2H), 2.97-2.88 (m, 1H), 2.33 (ddd, 15.3, 9.8, 5.4 Hz, 1H), 2.21 (ddd, 15.5, 9.2, 6.9 Hz, 1H), 2.12-2.00 (m, 2H), 1.97-1.77 (m, 5H), 1.69-1.62 (m, 1H), 1.61-1.23 (overlapping signals: m, 11H; 1.33, t, 7.4 Hz, 3H), 1.16-1.04 (overlapping signals: m, 1H; 1.09, s, 3H), 0.96 (d, 6.6 Hz, 3H), 0.73 (s, 3H);

¹⁹F NMR (470 MHz, CD₃OD) δ −76.94;

¹³C NMR (125 MHz, CD₃OD) δ 178.14, 70.94, 56.78, 56.60, 53.29, 53.25, 49.26 (from DEPT 135), 44.78, 44.36, 42.82, 41.10, 39.81, 36.69, 35.12, 34.23, 33.98, 32.39, 32.07, 29.62, 27.90, 23.12, 22.55, 18.95, 12.61, 9.78;

HRMS (ESI) m/z calcd for C₂₅H₄₃NO₃H⁺ 406.3316, found 406.3325.

### Example 9

### tert-Butyl N-qlycolyl-3-aza-7β-hydroxy-5β-cholan-24-oate (34a)

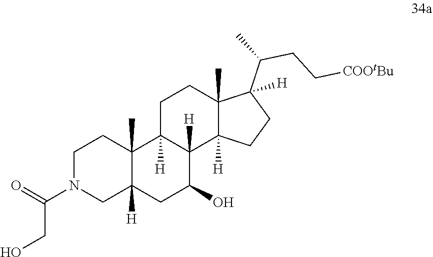

To a solution of the product of Example 2 (27a) (101 mg, 0.233 mmol) in dry dichloromethane (2 mL) was added sequentially glycolic acid (27.2 mg, 0.358 mmol; 1.5 equiv.), 1-hydroxybenzotriazole (HOBt; 34.9 mg, 0.258 mmol; 1.1 equiv.), N,N-diisopropylethylamine (0.089 mL, 0.511 mmol; 2.2 equiv.) and N-(3-dimethylaminopropyl)-N′-ethylcarbodiimide hydrochloride (EDCI; 59.9 mg, 0.313 mmol; 1.3 equiv.) at room temperature. After being stirred overnight the reaction was quenched with saturated NH₄Cl solution and diluted with water and dichloromethane. The mixture was extracted with dichloromethane (3×) and the combined organic phases were dried over MgSO₄ and concentrated. The residue was purified by automated column chromatography (silica gel, petroleum ether/ethyl acetate 2-80%) to yield 85.8 mg (75%) of 34a as a colourless foam.

¹H NMR (500 MHz, CDCl₃) δ 4.33-4.25 (m, 1H), 4.19-4.06 (m, 2H), 3.68-3.63 (m, 1H, OH), 3.57-3.46 (m, 1H), 3.24-3.16 (m, 1H), 3.11 (dd, 13.6, 3.8 Hz, 0.5H), 3.01 (td, 13.4, 2.0 Hz, 0.5H), 2.90 (ap t, 13.2 Hz, 0.5H), 2.73 (td, 13.4, 1.6 Hz, 0.5H), 2.26 (ddd, 15.3, 9.8, 5.3 Hz, 1H), 2.13 (ddd, 15.5, 9.3, 6.7 Hz, 1H), 2.06-2.00 (m, 1H), 1.97-1.87 (m, 1H), 1.87-1.68 (m, 4.5H), 1.68-1.59 (m, 1.5H), 1.55-1.33 (m, 8H; 1.44, s, 9H), 1.33-1.12 (m, 4H), 1.08 (dt, 9.6, 9.6 Hz, 1H), 1.02 (s, 3H), 0.92 (d, 6.5 Hz, 3H), 0.70 (s, 3H);

HRMS (ESI) m/z calcd for C₂₉H₄₉NO₅Na⁺ 514.3503, found 514.3503.

### Example 10

### N-Glycolyl-3-aza-7β-hydroxy-5β-cholan-24-oic acid (40a)

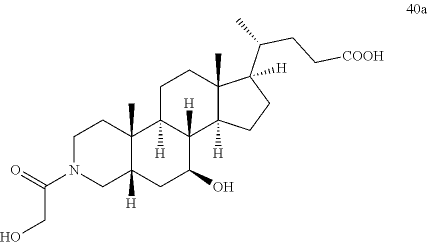

To a solution of the compound of Example 9 (34a) (85.8 mg, 0.175 mmol) in dry dichloromethane (6 mL) was added trifluoroacetic acid (4 mL) at 0° C. After being stirred for 2.5 h the solvent was evaporated in vacuo and the residue purified by automated column chromatography [silica gel, acetone (+1% acetic acid)/dichloromethane (+1% acetic acid) 0-40%]. The purified product was then co-evaporated with dichloromethane (3-4×) and methanol (1×) at 60° C. to give 45.5 mg (60%) of the product 40a as a colourless foam.

¹H NMR (500 MHz, CD₃OD) δ 4.26-4.12 (m, 3H), 3.48-3.38 (m, 1.5H), 3.38-3.27 (m, 1H), 3.08 (td, 13.4, 1.5 Hz, 0.5H), 2.96 (ap t, 13.1 Hz, 0.5H), 2.79-2.71 (m, 0.5H), 2.33 (ddd, 15.3, 9.9, 5.4 Hz, 1H), 2.20 (ddd, 15.5, 9.3, 6.8 Hz, 1H), 2.08-2.01 (m, 1H), 1.95-1.77 (m, 5H), 1.68-1.54 (m, 3H), 1.54-1.36 (m, 5H), 1.36-1.17 (m, 4.5H), 1.16-1.06 (m, 1.5H), 1.03 (s, 3H), 0.96 (d, 6.6 Hz, 3H), 0.73 (s, 3H);

¹³C NMR (125 MHz, CD₃OD) δ 178.16, 171.77, 71.57, 71.54, 61.23, 61.16, 57.23, 56.59, 45.82, 44.97, 44.80, 44.35, 43.86, 41.36, 40.81, 40.19, 38.80, 37.06, 36.71, 36.46, 36.06, 35.91, 35.05, 32.42, 32.08, 29.64, 27.93, 23.78, 23.74, 22.63, 22.60, 18.98, 12.69;

HRMS (ESI) m/z calcd for C₂₅H₄₁NO₅Na⁺ 458.2877, found 458.2877.

### Example 11

### N-[(2S)-2-amino-3-methylbutanoyl]-3-aza-7β-hydroxy-5β-cholan-24-oic acid hydrotrifluoroacetate (41a)

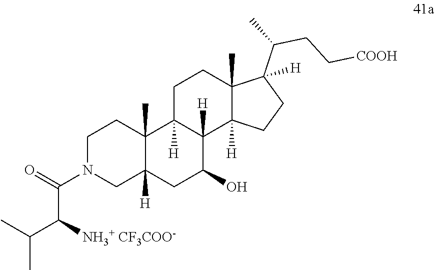

### A. tert-Butyl N-{(2S)-2-[(tert-butoxycarbonyl)amino]-3-methylbutanoyl}-3-aza-7β-hydroxy-5β-cholan-24-oate (35a)

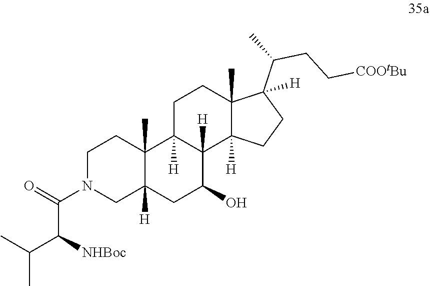

Deploying the same procedure as for Example 9, the compound of Example 2 (27a) (100 mg, 0.231 mmol) was dissolved in dry dichloromethane (2.5 mL) and sequentially treated with Boc-L-valine (Boc-Val-OH; 75.7 mg, 0.348 mmol), 1-hydroxybenzotriazole (HOBt; 34.6 mg, 0.256 mmol), N,N-diisopropylethylamine (0.089 mL, 0.511 mmol) and N-(3-dimethylaminopropyl)-N′-ethylcarbodiimide hydrochloride (EDCI; 58.3 mg, 0.304 mmol) to afford 122 mg (84%) of the product 35a as a colourless foam.

¹H NMR (500 MHz, CDCl₃) δ 5.38-5.30 (m, 1H, NH), 4.49-4.41 (m, 1H), 4.39-4.33 (m, 0.5H), 4.27 (dd, 13.6, 3.7 Hz, 0.5H), 3.69-3.63 (m, 0.5H), 3.63-3.57 (m, 0.5H), 3.56-3.47 (m, 1H), 3.25 (ap t, 13.1 Hz, 0.5H), 3.12 (td, 13.3, 1.5 Hz, 0.5H), 2.81 (ap t, 13.3 Hz, 0.5H), 2.61 (td, 13.3, 1.9 Hz, 0.5H), 2.26 (dddd, 15.2, 9.8, 5.3, 1.6 Hz, 1H), 2.13 (ddd, 15.4, 9.3, 6.8 Hz, 1H), 2.06-1.99 (m, 1H), 1.97-1.74 (m, 6H), 1.74-1.53 (m, 2H), 1.53-1.33 (m, 8H; 1.44, s, 9H; 1.432, s, 4.5H; 1.427, s, 4.5H), 1.33-1.12 (m, 4H), 1.11-1.05 (m, 1H), 1.02 (s, 1.5H), 0.99 (s, 1.5H), 0.96 (d, 6.7 Hz, 1.5H), 0.95 (d, 6.6 Hz, 1.5H), 0.93 (d, 6.5 Hz, 3H), 0.89 (d, 6.8 Hz, 1.5H), 0.85 (d, 6.7 Hz, 1.5H), 0.696 (s, 1.5H), 0.694 (s, 1.5H);

HRMS (ESI) m/z calcd for C₃₇H₆₄N₂O₆H⁺633.4837, found 633.4839.

### B. N-[(2S)-2-amino-3-methylbutanoyl]-3-aza-7β-hydroxy-5β-cholan-24-oic acid hydrotrifluoroacetate (41a)

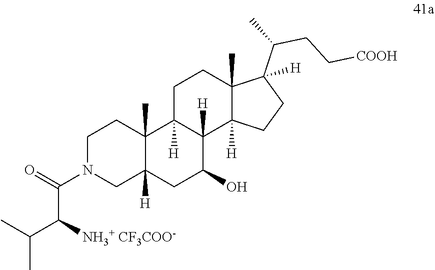

Applying the same method of deprotection as for Example 10, the product of step A (35a) (122 mg, 0.193 mmol) dissolved in dry dichloromethane (5 mL) was treated with trifluoroacetic acid (4 mL) at 0° C. for 2.5 h and the crude product was purified by automated column chromatography [C18 silica gel, acetonitrile/water (+0.5% trifluoroacetic acid) 0-50%]. The purified product was co-evaporated with dichloromethane (3-4×) and methanol (1×) at 60° C. to give 67.9 mg (60%) of 41a as a colourless foam.

¹H NMR (500 MHz, CD₃OD; mixture of rotamers) δ 4.35 (d, 4.9 Hz, 0.5H), 4.32-4.26 (m, 0.5H), 4.24 (d, 5.0 Hz, 0.5H), 4.22-4.16 (m, 0.5H), 3.65-3.59 (m, 0.5H), 3.59-3.53 (m, 0.5H), 3.49-3.38 (m, 1.5H), 3.21 (td, 13.3, 1.5 Hz, 0.5H), 2.99 (ap t, 13.3 Hz, 0.5H), 2.79 (td, 13.3, 1.7 Hz, 0.5H), 2.33 (ddd, 15.3, 9.9, 5.4 Hz, 1H), 2.25-2.12 (m, 2H), 2.09-2.02 (m, 1H), 1.98-1.77 (m, 5H), 1.74-1.59 (m, 2.5H), 1.59-1.37 (m, 5.5H), 1.37-1.16 (m, 5H), 1.16-1.07 (overlapping signals: m, 1H; 1.10, d, 7.3 Hz, 1.5H; 1.09, d, 7.3 Hz, 1.5H), 1.05 (s, 1.5H), 1.03 (s, 1.5H), 1.01 (d, 7.0 Hz, 1.5H), 0.99 (d, 7.0 Hz, 1.5H), 0.96 (d, 6.5 Hz, 3H), 0.74 (s, 3H);

¹⁹F NMR (470 MHz, CD₃OD) δ −77.00;

¹³C NMR (125 MHz, CD₃OD) δ 178.17, 168.04, 167.89, 71.57, 71.52, 57.28, 57.10, 56.59, 56.34, 48.01, 45.75, 44.81, 44.45, 44.37, 44.18, 42.36, 41.38, 41.32, 40.46, 40.15, 39.21, 37.22, 36.71, 36.47, 36.04, 35.78, 35.19, 34.95, 32.41, 32.06, 30.94, 29.64, 27.94, 23.78, 23.67, 22.64, 22.58, 19.31, 19.10, 18.95, 17.04, 16.82, 12.66; HRMS (ESI) m/z calcd for C₂₈H₄₈N₂O₄H⁺ 477.3687, found 477.3696.

### Example 12

### N-[(2S,3S)-2-Amino-3-methylpentanoyl]-3-aza-7β-hydroxy-5β-cholan-24-oic acid hydrotrifluoroacetate (42a)

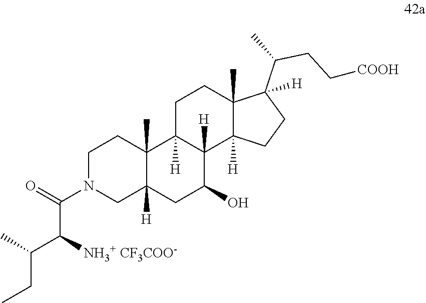

### A. tert-Butyl N-{(2S,3S)-2-[(tert-butoxycarbonyl)amino]-3-methylpentanoyl}-3-aza-7p-hydroxy-5β-cholan-24-oate (36a)

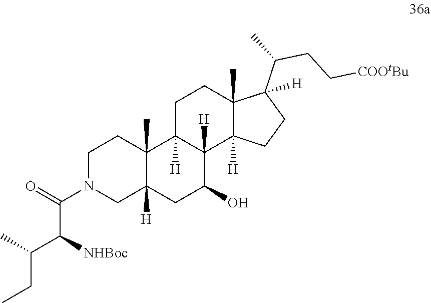

Using the same protocol as for Example 9, the compound of Example 2 (27a) (101 mg, 0.233 mmol) was dissolved in dry dichloromethane (2.5 mL) and sequentially treated with Boc-L-isoleucine (Boc-Ile-OH; 82.4 mg, 0.356 mmol), 1-hydroxybenzotriazole (35.0 mg, 0.259 mmol), N,N-diisopropylethylamine (0.09 mL, 0.517 mmol) and N-(3-dimethylaminopropyl)-N′-ethylcarbodiimide hydrochloride (59.0 mg, 0.308 mmol) to afford 129 mg (86%) of 36a as a colourless foam.

¹H NMR (500 MHz, CDCl₃) δ 5.37-5.30 (m, 1H, NH), 4.51-4.43 (m, 1H), 4.39-4.32 (m, 0.5H), 4.28 (dd, 13.6, 3.7 Hz, 0.5H), 3.75-3.62 (m, 1H), 3.56-3.47 (m, 1H), 3.25 (ap t, 13.0 Hz, 0.5H), 3.18-3.09 (m, 0.5H), 2.82 (apt, 13.3 Hz, 0.5H), 2.62 (td, 13.4, 1.9 Hz, 0.5H), 2.26 (dddd, 15.2, 9.8, 5.3, 1.8 Hz, 1H), 2.13 (ddd, 15.4, 9.3, 6.7 Hz, 1H), 2.06-1.99 (m, 1H), 1.97-1.61 (m, 7.5H), 1.60-1.14 (m, 13.5H; 1.44, s, 9H; 1.424, s, 4.5H; 1.422, s, 4.5H), 1.14-1.04 (m, 2H), 1.02 (s, 1.5H), 0.99 (s, 1.5H), 0.95-0.85 (overlapping signals: m, 3H, 1.25, d, 6.6 Hz, 3H; 0.88, t, 7.4 Hz, 1.5H; 0.87, t, 7.4 Hz, 1.5H), 0.697 (s, 1.5H), 0.694 (s, 1.5H);

HRMS (ESI) m/z calcd for C₃₈H₆₆N₂O₆Na⁺669.4813, found 669.4822.

### B. N-[(2S,3S)-2-Amino-3-methylpentanoyl]-3-aza-7β-hydroxy-5β-cholan-24-oic acid hydrotrifluoroacetate (42a)

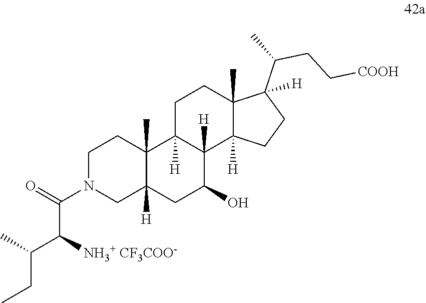

Applying the same method as for Example 11, step B, the compound of step A (36a) (116 mg, 0.179 mmol) dissolved in dry dichloromethane (5 mL) was reacted with trifluoroacetic acid (4 mL) at 0° C. for 2.5 h to give 74.2 mg (68%) of the product 42a as a colourless foam.

¹H NMR (500 MHz, CD₃OD; mixture of rotamers) δ 4.36 (d, 4.9 Hz, 0.5H), 4.32-4.23 (overlapping signals: m, 0.5H; 4.25, d, 4.9 Hz, 0.5H), 4.19 (dd, 13.7, 3.4 Hz, 0.5H), 3.64-3.52 m, 1H), 3.50-3.38 (m, 1.5H), 3.21 (td, 13.3, 1.6 Hz, 0.5H), 2.99 (ap t, 13.3 Hz, 0.5H), 2.79 (td, 13.3, 1.9 Hz, 0.5H), 2.33 (ddd, 15.3, 9.8, 5.4 Hz, 1H), 2.20 (ddd, 15.5, 9.3, 6.8 Hz, 1H), 2.09-2.02 (m, 1H), 1.99-1.77 (m, 6H), 1.73-1.58 (m, 2.5H), 1.58-1.37 (m, 6.5H), 1.37-1.09 (m, 7H), 1.08 (d, 7.1 Hz, 1.5H), 1.06 (d, 7.2 Hz, 1.5H), 1.05 (s, 1.5H), 1.03 (s, 1.5H), 1.00-0.94 (m, 6H), 0.74 (s, 3H);

¹⁹F NMR (470 MHz, CD₃OD) δ −76.95;

¹³C NMR (125 MHz, CD₃OD; mixture of rotamers) δ 178.16, 168.02, 167.87, 71.57, 71.51, 57.28, 57.08, 56.64, 56.58, 56.35, 56.01, 48.12, 45.77, 44.81, 44.52, 44.44, 44.38, 44.24, 42.40, 41.38, 41.31, 40.49, 40.15, 39.24, 37.71, 37.67, 37.33, 36.71, 36.46, 36.04, 35.81, 35.20, 34.97, 32.41, 32.06, 29.64, 27.95, 27.93, 24.68, 24.51, 23.78, 23.73, 22.64, 22.58, 18.96, 15.81, 15.46, 12.68, 12.65, 12.07;

HRMS (ESI) m/z calcd for C₂₉H₅₀N₂O₄H⁺491.3843, found. 491.3857.

### Example 13

### N-[(2S)-2-Amino-4-methylpentanoyl]-3-aza-7β-hydroxy-5β-cholan-24-oic acid hydrotrifluoroacetate (43a)

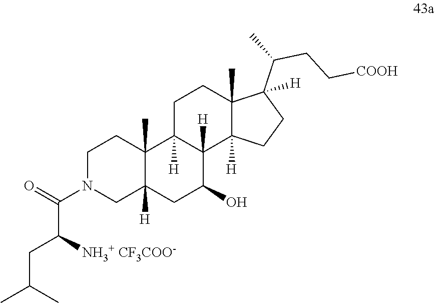

### A. tert-Butyl N-{(2S)-2-[(tert-butoxycarbonyl)amino]-4-methylpentanoyl}-3-aza-7β-hydroxy-5β-cholan-24-oate (37a)

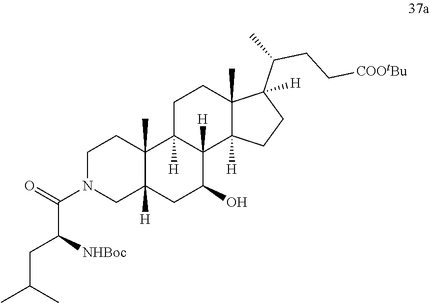

Deploying the same procedure as for Example 9, the compound of Example 2 (27a) (102 mg, 0.235 mmol) was dissolved in dry dichloromethane (2.5 mL) and reacted with Boc-L-leucine (Boc-Leu-OH; 81.7 mg, 0.353 mmol), 1-hydroxybenzotriazole (35.3 mg, 0.261 mmol), N,N-diisopropylethylamine (0.09 mL, 0.517 mmol) and N-(3-dimethylaminopropyl)-N′-ethylcarbodiimide hydrochloride (59.4 mg, 0.310 mmol) to give 134 mg (88%) of the product 37a as a colourless foam.

¹H NMR (500 MHz, CDCl₃) δ 5.31 (s, 0.5H, NH), 5.29 (s, 0.5H, NH), 4.68-4.59 (m, 1H), 4.36-4.29 (m, 0.5H), 4.25 (dd, 13.7, 3.8 Hz, 0.5H), 3.66-3.56 (m, 1H), 3.56-3.46 (m, 1H), 3.23 (ap t, 13.1 Hz, 0.5H), 3.17-3.08 (m, 0.5H), 2.81 (ap t, 13.2 Hz, 0.5H), 2.59 (td, 13.4, 1.9 Hz, 0.5H), 2.26 (dddd, 15.2, 9.8, 5.3, 1.5 Hz, 1H), 2.13 (ddd, 15.4, 9.3, 6.7 Hz, 1H), 2.06-1.99 (m, 1H), 1.97-1.55 (m, 8H), 1.55-1.12 (m, 14H; 1.44, s, 9H; 1.428, s, 4.5H; 1.426, s, 4.5H), 1.12-1.04 (m, 1H), 1.02 (s, 1.5H), 1.01-0.96 (overlapping signals: m, 3H; 1.00, s, 1.5H); 0.94-0.90 (m, 6H), 0.70 (s, 3H);

HRMS (ESI) m/z calcd for C₃₈H₆₆N₂O₆Na⁺ 669.4813, found 669.4803.

### B. N-[(2S)-2-Amino-4-methylpentanoyl]-3-aza-7β-hydroxy-5β-cholan-24-oic acid hydrotrifluoroacetate (43a)

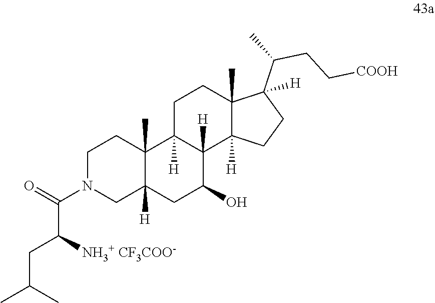

Following the same method as for Example 11, step B, the compound of step A (37a) (120 mg, 0.186 mmol) was dissolved in dry dichloromethane (5 mL) and stirred in the presence of trifluoroacetic acid (4 mL) to give rise to 73.3 mg (65%) of the product 43a as a colourless foam.

¹H NMR (500 MHz, CD₃OD; mixture of rotamers) δ 4.43 (dd, 9.2, 3.7 Hz, 0.5H), 4.35 (dd, 9.1, 3.5 Hz, 0.5H), 4.29-4.22 (m, 0.5H), 4.17 (dd, 13.6, 3.6 Hz, 0.5H), 3.52-3.38 (m, 2.5H), 3.28-3.19 (m, 0.5H), 3.00 (ap t, 13.3 Hz, 0.5H), 2.77 (td, 13.3, 1.9 Hz, 0.5H), 2.33 (ddd, 15.3, 9.8, 5.4 Hz, 1H), 2.20 (ddd, 15.5, 9.2, 6.9 Hz, 1H), 2.10-2.02 (m, 1H), 1.99-1.78 (m, 5H), 1.78-1.54 (m, 6H), 1.54-1.15 (m, 10H), 1.15-1.07 (m, 1H), 1.06 (s, 1.5H), 1.05-0.99 (overlapping signals: 1.04, s, 1.5H; m, 6H), 0.96 (d, 6.5 Hz, 3H), 0.74 (s, 3H);

¹⁹F NMR (470 MHz, CD₃OD) δ −76.95;

¹³C NMR (125 MHz, CD₃OD; mixture of rotamers) δ 178.16, 168.82, 168.73, 71.54, 57.27, 57.10, 56.64, 56.61, 50.46, 47.65, 45.44, 44.81, 44.41, 44.38, 44.25, 44.11, 42.21, 41.55, 41.38, 41.32, 41.02, 40.45, 40.17, 39.18, 37.06, 36.71, 36.42, 35.99, 35.98, 35.12, 35.02, 32.41, 32.07, 29.63, 27.95, 25.51, 25.46, 23.77, 23.64, 23.61, 23.59, 22.64, 22.58, 21.82, 21.72, 18.96, 12.67;

HRMS (ESI) m/z calcd for C₂₉H₅₀N₂O₄H⁺ 491.3843, found 491.3857

### Example 14

### N-[(2S)-2-Amino-3-(1H-indol-3-yl)propanoyl]-3-aza-7β-hydroxy-5β-cholan-24-oic acid hydrotrifluoroacetate (44a)

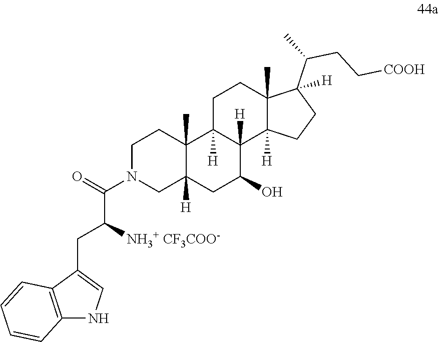

### A. tert-Butyl N-{(2S)-2-[(tert-butoxycarbonyl)amino]-3-[1H-indol-3-yl]propanoyl}-3-aza-7β-hydroxy-5β-cholan-24-oate (38a)

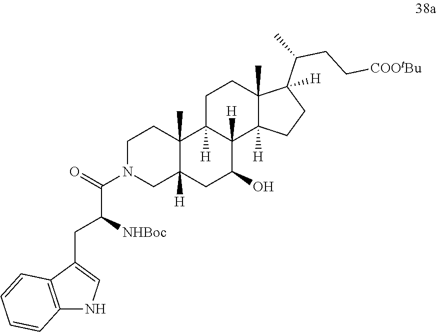

Applying the same protocol as for Example 9, the compound of Example 2 (27a) (90.2 mg, 0.208 mmol) was dissolved in dry dichloromethane (2.5 mL) and treated with Na-Boc-L-tryptophan (Boc-Trp-OH; 95.3 mg, 0.313 mmol), 1-hydroxybenzotriazole (31.1 mg, 0.230 mmol), N,N-diisopropylethylamine (0.08 mL, 0.459 mmol) and N-(3-dimethylaminopropyl)-N′-ethylcarbodiimide hydrochloride (51.9 mg, 0.271 mmol) to afford 122 mg (82%) of the product 38a as a colourless oil.

¹H NMR (500 MHz, CDCl₃; mixture of rotamers) δ 8.20 (s, 0.5H, NH), 8.10 (s, 0.5H, NH), 7.69-7.64 (m, 0.5H), 7.58 (d, 7.8 Hz, 0.5H), 7.36-7.30 (m, 1H), 7.21-7.08 (m, 2H), 7.08-7.03 (m, 1H), 5.66 (d, 8.4 Hz, 0.5H, NH), 5.48 (d, 8.7 Hz, 0.5H, NH), 4.99-4.92 (m, 0.5H), 4.92-4.85 (m, 0.5H), 4.16-4.07 (m, 1H), 3.44-3.35 (m, 0.5H), 3.29-3.19 (m, 1H), 3.18-3.12 (m, 1H), 3.12-3.01 (overlapping signals: 3.08, dd, 14.0, 9.5 Hz, 0.5H; m, 0.5H), 2.85-2.75 (m, 1H), 2.61 (ap t, 13.2 Hz, 0.5H), 2.34-2.06 (overlapping signals: 2.29, ddd, 15.3, 10.0, 5.4 Hz, 0.5H; m, 2H), 2.01-1.84 (m, 2H), 1.84-1.49 (m, 4H), 1.49-0.97 (m, 14H; 1.46, s, 4.5H; 1.45, s, 4.5H; 1.44, s, 4.5H; 1.43, s, 4.5H), 0.94 (d, 6.5 Hz, 1.5H), 0.91-0.82 (overlapping signals: 0.88, d, 6.5 Hz, 1.5H; m, 0.5H; 0.84, s, 1.5H), 0.82-0.73 (m, 0.5H); 0.65-0.59 (overlapping singlets: 0.63, 1.5H; 0.62, 3H), −0.36 (td, 13.4, 2.8 Hz, 0.5H);

HRMS (ESI) m/z calcd for C₄₃H₆₅N₃O₆Na⁺ 742.4766, found 742.4764.

### B. N-[(2S)-2-Amino-3-(1H-indol-3-yl)propanoyl]-3-aza-7β-hydroxy-5β-cholan-24-oic acid hydrotrifluoroacetate (44a)

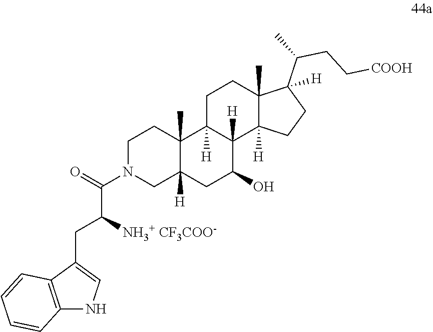

Employing the same reaction conditions as Example 11, step B, the compound of step A (38a) (122 mg, 0.169 mmol) dissolved in dry dichloromethane (5 mL) was treated with trifluoroacetic acid (4 mL) at 0° C. for 2.5 h to give 83.7 mg (73%) of the product 44a as a colourless foam.

¹H NMR (500 MHz, CD₃OD; mixture of rotamers) δ 7.55-7.51 (m, 0.5H), 7.46-7.35 (m, 1.5H), 7.22 (s, 0.5H), 7.20 (s, 0.5H), 7.19-7.07 (m, 1.5H), 7.06-7.02 (m, 0.5H), 4.65-4.57 (m, 1H), 4.04-3.96 (m, 1H), 3.36-3.20 (overlapping signals: m, 2H; 3.23, dd, 13.7, 10.4 Hz, 0.5H), 3.15-3.08 (m, 0.5H), 2.95 (dd, 13.4, 3.8 Hz, 0.5H), 2.84 (td, 13.2, 1.9 Hz, 0.5H), 2.77-2.65 (m, 1H), 2.38 (ddd, 15.3, 9.8, 5.4 Hz, 0.5H), 2.33-2.13 (m, 2H), 2.00-1.91 (m, 1.5H), 1.91-1.74 (m, 2.5H), 1.64-1.00 (m, 14H), 1.00-0.94 (overlapping signals: m, 1H; 0.99, d, 6.5 Hz, 1.5H), 0.91 (d, 6.5 Hz, 1.5H) 0.86 (s, 1.5H), 0.69-0.63 (overlapping signals: m, 1H; 0.66, d, 6.5 Hz, 3H), 0.60 (s, 1.5H), −0.52 (td, 13.8, 3.1 Hz, 0.5H);

¹⁹F NMR (470 MHz, CD₃OD) δ −76.94;

¹³C NMR (125 MHz, CD₃OD; mixture of rotamers) δ 178.20, 178.14, 169.37, 168.65, 138.15, 138.05, 128.81, 128.72, 125.76, 125.50, 123.33, 123.05, 120.53, 120.45, 119.31, 119.11, 112.99, 112.90, 108.63, 108.30, 71.41, 71.07, 57.21, 56.86, 56.77, 56.58, 52.13, 51.27, 46.43, 44.77, 44.73, 44.22, 44.09, 43.98, 43.86, 43.13, 42.59, 41.30, 41.19, 40.16, 40.11, 38.69, 36.75, 36.67, 35.94, 35.85, 35.57, 35.21, 34.11, 32.45, 32.37, 32.11, 32.04, 29.66, 29.57, 29.26, 29.17, 27.87, 27.77, 23.22, 23.03, 22.44, 22.34, 19.00, 18.91, 12.61;

HRMS (ESI) m/z calcd for C₃₄H₄₉N₃O₄H⁺ 564.3796, found 564.3799.

### Example 15

### N—[R(2S)-2-Amino-3-(4-hydroxyphenyl)propanoyl]-3-aza-7β-hydroxy-5β-cholan-24-oic acid hydrotrifluoroacetate (45a)

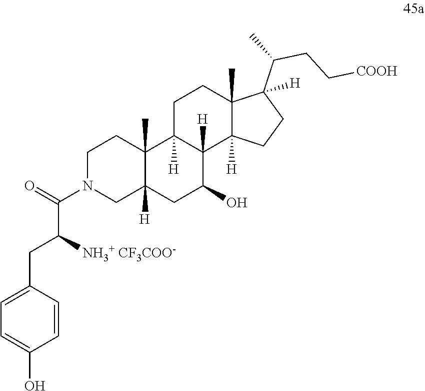

### A. tert-Butyl N-{(2S)-2-[(tert-butoxycarbonyl)amino]-3-[4-hydroxyphenyl]propanoyl}-3-aza-7β-hydroxy-5β-cholan-24-oate (39a)

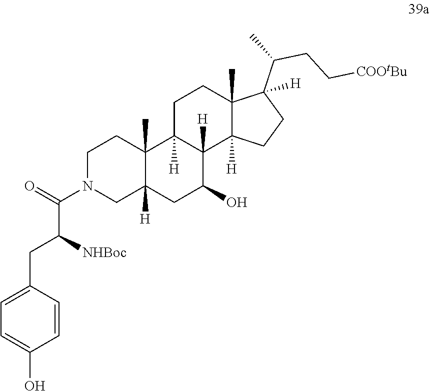

Using the same method as for Example 9, the compound of Example 2 (27a) (91.9 mg, 0.212 mmol) was dissolved in dry dichloromethane (2.5 mL) and reacted with Boc-L-tyrosine (Boc-Tyr-OH; 91.0 mg, 0.324 mmol), 1-hydroxybenzotriazole (31.8 mg, 0.235 mmol), N,N-diisopropylethylamine (0.08 mL, 0.459 mmol) and N-(3-dimethylaminopropyl)-N′-ethylcarbodiimide hydrochloride (53.3 mg, 0.278 mmol) to give 110 mg (75%) of 39a as a colourless foam.

¹H NMR (500 MHz, CDCl₃; mixture of rotamers) δ 7.04 (AA′XX′, J_{AX}=8.3 Hz, 1H), 7.01 (AA′XX′, J_{AX}=8.4 Hz, 1H), 6.78-6.72 (m, 2H), 5.56 (d, 8.6 Hz, 0.5H, NH), 5.41 (d, 8.7 Hz, 1H, NH), 4.85-4.73 (m, 1H), 4.25-4.15 (m, 1H), 3.49-3.39 (m, 1H), 3.30-3.18 (m, 1H), 3.01-2.79 (overlapping signals: m, 2H; 2.82, dd, 13.3, 8.7 Hz, 0.5H), 2.71 (ap t, 13.2 Hz, 0.5H), 2.52 (ap t, 12.9 Hz, 0.5H), 2.32-2.20 (m, 1.5H), 2.18-2.08 (m, 1H), 2.05-1.94 (m, 1H), 1.94-1.84 (m, 1H), 1.84-1.59 (m, 4H), 1.56-1.00 (m, 15.5H; 1.45, s, 4.5H; 1.44, s, 4.5H; 1.43, s, 4.5H; 1.42, s, 4.5H), 0.95-0.89 (overlapping signals: 0.93, d, 6.5 Hz, 1.5H; 0.93, s, 1.5H; 0.91, d, 6.6 Hz, 1.5H), 0.84 (s, 1.5H), 0.67 (s, 1.5H), 0.66 (s, 1.5H), 0.21 (td, 13.4, 2.8 Hz, 0.5H);

HRMS (ESI) m/z calcd for C₄₁H₆₄N₂O₇H⁺697.4786, found 697.4785.

### B. N-[(2S)-2-Amino-3-(4-hydroxyphenyl)propanoyl]-3-aza-7β-hydroxy-5β-cholan-24-oic acid hydrotrifluoroacetate (45a)

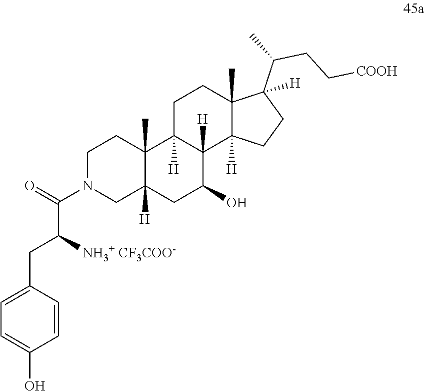

Using the same method as for Example 11, step B, the compound of step A (39a) (110 mg, 0.158 mmol) was dissolved in dry dichloromethane (5 mL) and reacted with trifluoroacetic acid (4 mL) at 0° C. for 2.5 h to yield 56.0 mg (54%) of the product 45a as a colourless foam.

¹H NMR (500 MHz, CD₃OD; mixture of rotamers) δ 7.07 (AA′XX′, J_{AX}=8.5 Hz, 1H), 7.05 (AA′XX′, J_{AX}=8.5 Hz, 1H), 6.79 (AA′XX′, J_{AX}=8.6 Hz, 1H), 6.76 (AA′XX′, J_{AX}=8.6 Hz, 1H), 4.60 (dd, 9.9, 5.7 Hz, 0.5H), 4.54 (dd, 9.7, 5.8 Hz, 0.5H), 4.17-4.09 (m, 1H), 3.40-3.26 (m, 1H), 3.13-2.82 (m, 4H), 2.58 (td, 13.2, 2.2 Hz, 0.5H), 2.39-2.27 (m, 1H), 2.26-2.15 (m, 1H), 2.08-1.98 (m, 1.5H), 1.95-1.68 (m, 4.5H), 1.59 (ddd, 13.6, 5.0, 2.1 Hz, 0.5H), 1.53-1.04 (m, 13.5H), 0.99-0.92 (overlapping signals: 0.97, d, 6.5 Hz, 1.5H; 0.95, s, 1.5H; 0.94, d, 6.6 Hz, 1.5H), 0.85 (s, 1.5H), 0.704 (s, 1.5H), 0.698 (s, 1.5H), 0.05 (td, 13.8, 4.2 Hz, 0.5H); ¹⁹F NMR (470 MHz, CD₃OD) δ −76.98; ¹³C NMR (125 MHz, CD₃OD; mixture of rotamers) δ 178.22, 178.16, 168.60, 168.00, 158.73, 158.60, 132.12, 131.98, 125.88, 125.85, 117.03, 116.68, 71.46, 71.33, 57.24, 56.89, 56.60, 56.35, 52.31, 51.74, 46.72, 44.77, 44.40, 44.32, 44.27, 44.05, 43.79, 42.63, 41.34, 40.54, 40.24, 38.53, 38.49, 38.27, 36.72, 36.69, 36.12, 35.99, 35.85, 35.68, 34.56, 34.51, 32.44, 32.39, 32.06, 29.65, 29.60, 27.91, 27.87, 23.46, 23.27, 22.54, 22.47, 18.97, 18.94, 12.67, 12.65;

HRMS (ESI) m/z calcd for C₃₂H₄₈N₂O₅H⁺ 541.3636, found 541.3632

### Example 16

### tert-Butyl N-(benzyloxycarbonyl)-3-aza-7β-hydroxy-25-homo-5β-cholan-25-oate (25b)

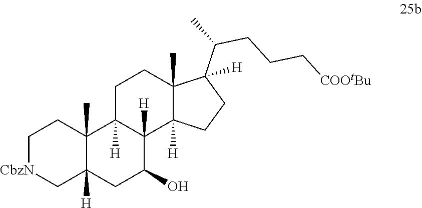

### A. Methyl 3α,7β-3,7-dihydroxy-24-nor-5β-cholan-23-oate

The title compound was prepared from ursodeoxycholic acid by the method of D'Amore et al 2014.

### B. Methyl 3α,7β-bis(tert-butyldimethylsilyloxy)-24-nor-5β-cholan-23-oate (3)

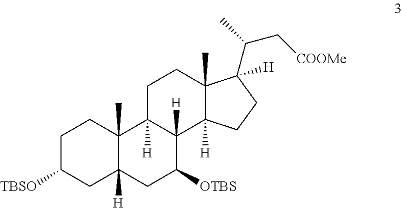

To a solution of the product of step A (diol 2; 37.8 g, 96.3 mmol) in dry DMF (1 L) was added imidazole (33.4 g, 491 mmol) and tert-butyl(chloro)dimethylsilane (60.3 g, 400 mmol) at room temperature. After being stirred at 60° C. overnight the reaction was diluted with water and extracted with ethyl acetate (3×). The combined organic layers were washed with water (2×) and brine, dried over MgSO₄ and concentrated. The crude product was purified by flash column chromatography (silica gel, ethyl acetate/petroleum ether 1:19, 1:9 and 1:6) to yield 53.6 g (90%) of the product 3 as a colourless amorphous solid.

¹H NMR (500 MHz, CDCl₃) δ 3.71-3.62 (overlapping signals: m, 1H; 3.66, s, 3H), 3.52 (tt, 10.7, 4.7 Hz, 1H), 2.43 (dd, 14.5, 3.3 Hz, 1H), 2.03-1.86 (m, 3H), 1.86-1.71 (m, 4H), 1.64 (dt, 11.1, 12.9 Hz, 1H), 1.58-1.33 (m, 8H), 1.32-1.20 (m, 3H), 1.18-1.00 (m, 3H), 1.00-0.91 (overlapping signals: m, 1H; 0.97, d, 6.5 Hz, 3H, 0.92, s, 3H), 0.893 (s, 9H), 0.885 (s, 9H), 0.68 (s, 3H), 0.060 (s, 3H), 0.057 (s, 3H), 0.049 (s, 3H), 0.046 (s, 3H);

HRMS (ESI) m/z calcd for C₃₆H₆₈O₄Si₂Na⁺ 643.4548, found 643.4553.

Procedure in: Yan, S.; Ding, N.; Zhang, W.; Wang, P.; Li, Y.; Li, M. Carbohydr. Research 2012, 354, 6-20.

### C. 3α,7β-bis(tert-Butyldimethylsilyloxy)-24-nor-5β-cholan-23-ol (4)

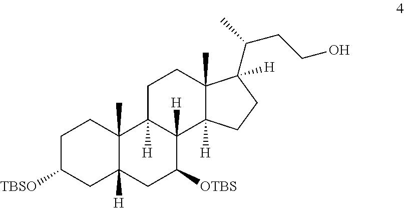

To a solution of the product of step B, the methyl ester 3 (53.5 g, 86.1 mmol) in dry THF (2 L) and methanol (25 mL, 617 mmol) was added lithium borohydride (13.3 g, 611 mmol) at 0° C. After 30 min the ice bath was removed and the reaction was stirred at room temperature overnight. Subsequently, the reaction the reaction was carefully quenched with 1 M sodium hydroxide solution (1 L). The organic phase was separated and the aqueous phase was extracted with ethyl acetate (2×). The organic phases were combined, washed with water and brine, dried over MgSO₄ and concentrated. The crude product was purified by flash column chromatography (silica gel, ethyl acetate 1:19, 1:9 and 1:4) to 50.0 g (98%) of the product, alcohol 4, as a dry colourless gel.

¹H NMR (500 MHz, CDCl₃) δ 3.74-3.60 (m, 3H), 3.52 (tt, 10.6, 4.7 Hz, 1H), 1.96 (dt, 12.5, 3.2 Hz, 1H), 1.86-1.69 (m, 5H), 1.64 (dt, 11.2, 12.9 Hz, 1H), 1.59-1.19 (m, 13H), 1.17-1.00 (m, 4H), 1.00-0.91 (overlapping signals: m, 1H; 0.95, d, 6.6 Hz, 3H; 0.92, s, 3H), 0.894 (s, 9H), 0.889 (s, 9H), 0.66 (s, 3H), 0.061 (s, 3H), 0.059 (s, 3H), 0.054 (s, 3H), 0.049 (s, 3H);

HRMS (ESI) m/z calcd for C₃₅H₆₈O₃Si₂Na⁺ 615.4599, found 615.4609.

### D. 3α,7β-bis(tert-Butyldimethylsilyloxy)-24-nor-5β-cholan-23-al (5)

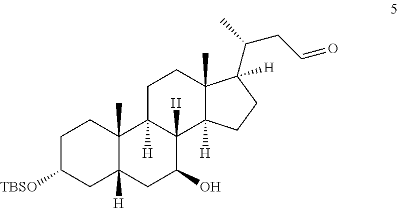

To a solution of oxalyl chloride (5.32 mL, 62.9 mmol) in dry THF (400 mL) was added dropwise dry DMSO (4.75 mL, 66.8 mmol) at −78° C. After 20 min a solution of 4, the product of step C (23.3 g, 39.3 mmol) in dry THF (100 mL) was introduced dropwise via cannula. Then, the reaction mixture was allowed to warm to −60° C. before dry triethylamine (16.4 mL, 118 mmol) was added and the reaction was allowed to slowly warm to room temperature. Water was added and the aqueous phase was extracted with ethyl acetate (3×). The combined organic phases were washed with brine, dried over MgSO₄ and concentrated. The crude product was purified by automated column chromatography (silica gel, ethyl acetate/petroleum ether 2-10%) to afford 19.2 g (83%) of the required product, 5, as an amorphous, colourless solid.

¹H NMR (500 MHz, CDCl₃) δ 9.75 (dd, 3.4, 1.4 Hz, 1H), 3.70-3.63 (m, 1H), 3.52 (tt, 10.7, 4.7 Hz, 1H), 2.46 (ddd, 15.9, 3.1, 1.0 Hz, 1H), 2.16 (ddd, 15.9, 9.2, 3.4 Hz, 1H), 2.07-1.98 (m, 1H), 1.96 (dt, 12.5, 3.3 Hz, 1H), 1.88-1.72 (m, 4H), 1.64 (dt, 10.9, 13.0 Hz, 1H), 1.58-1.35 (m, 8H), 1.35-1.20 (m, 3H), 1.20-1.06 (m, 3H), 1.03-0.91 (overlapping signals: 1.01, d, 6.6 Hz, 3H; m, 1H; 0.93, s, 3H), 0.894 (s, 9H), 0.886 (s, 9H), 0.70 (s, 3H), 0.062 (s, 3H), 0.059 (s, 3H), 0.050 (s, 3H), 0.047 (s, 3H);

HRMS (ESI) m/z calcd for C₃₅H₆₆O₃Si₂Na⁺ 613.4443, found 613.4448.

### E. Methyl 3α,7β-bis(tert-butyldimethylsilyloxy)-25-homo-5β-cholan-23-en-25-oate (7a) and tert-butyl 3α,7β-bis(tert-butyldimethylsilyloxy)-25-homo-5β-cholan-23-en-25-oate (7b)

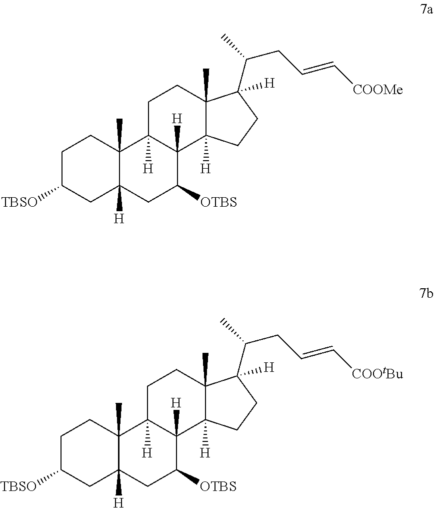

### Methyl Ester

To a solution of aldehyde 5, the product of step D (8.48 g, 14.3 mmol) in dry dichloromethane (188 mL) was added methyl (triphenylphosphoranylidene) acetate (14.9 g, 44.6 mmol) at room temperature. After being stirred overnight the reaction was concentrated to small volume and directly loaded onto a high performance cartridge packed with silica gel. Gradient elution with ethyl acetate and petroleum ether (0-15%) using an automated chromatography system yielded 8.74 g (94%) of the desired methyl ester product 7a as an amorphous, colourless solid.

¹H NMR (500 MHz, CDCl₃) δ 6.95 (ddd, 15.5, 8.8, 6.6 Hz, 1H), 5.84-5.78 (m, 1H), 3.73 (s, 3H), 3.70-3.63 (m, 1H), 3.52 (tt, 10.7, 4.7 Hz, 1H), 2.33-2.26 (m, 1H), 2.01-1.91 (m, 2H), 1.87-1.71 (m, 4H), 1.64 (dt, 11.0, 13.1 Hz, 1H), 1.61-1.34 (m, 9H), 1.34-1.20 (m, 3H), 1.18-1.00 (m, 3H), 1.00-0.91 (overlapping signals: m, 1H; 0.94, d, 6.7 Hz, 3H; 0.92, s, 3H), 0.893 (s, 9H), 0.890 (s, 9H), 0.66 (s, 3H), 0.059 (s, 3H), 0.056 (s, 6H), 0.050 (s, 3H);

HRMS (ESI) m/z calcd for C₃₈H₇₀O₄Si₂Na⁺ 669.4705, found 669.4708.

### Tert-Butyl Ester

Employing the same procedure as for the preparation of methyl ester 7a aldehyde 5 (19.2 g, 32.5 mmol) was dissolved in dry dichloromethane (520 mL) and reacted with (tert-butoxycarbonylmethylene)triphenylphosphorane (43.0 g, 114 mmol) at room temperature for 72 h to give 21.8 g (97%) of the tert-butyl ester 7b as a colourless foam.

¹H NMR (500 MHz, CDCl₃) δ 6.83 (ddd, 15.4, 8.7, 6.5 Hz, 1H), 5.75-5.69 (m, 1H), 3.70-3.63 (m, 1H), 3.52 (tt, 10.6, 4.7 Hz, 1H), 2.31-2.24 (m, 1H), 1.97-1.87 (m, 2H), 1.86-1.71 (m, 4H), 1.64 (dt, 11.0, 13.0 Hz, 1H), 1.59-1.32 (m, 9H), 1.49 (s, 9H), 1.32-1.19 (m, 3H), 1.18-1.00 (m, 3H), 1.00-0.91 (overlapping signals: m, 4H; 0.92, s, 3H), 0.893 (s, 9H), 0.890 (s, 9H), 0.65 (s, 3H), 0.059 (s, 3H), 0.056 (s, 6H), 0.050 (s, 3H);

HRMS (ESI) m/z calcd for C₄₁H₇₆O₄Si₂Na⁺ 711.5174, found 711.5175.

### F. Methyl 3α,7β-dihydroxy-25-homo-5β-cholan-25-oate (9a) and tert-butyl 3α,7β-dihydroxy-25-homo-5β-cholan-25-oate (9b)

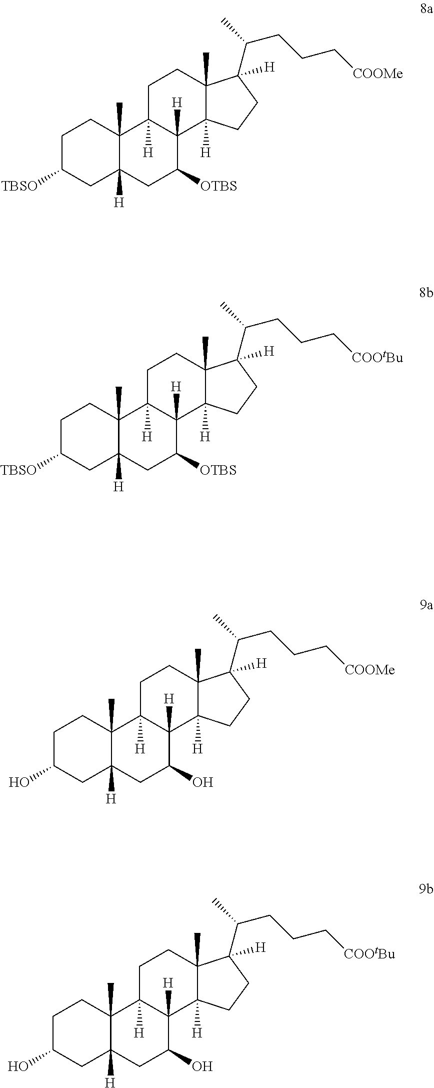

### Methyl Ester

To a solution of alkene 7a, the product of step E (8.07 g, 12.5 mmol) in ethyl acetate (220 mL) was added 10% palladium on charcoal (673 mg) and the atmosphere was exchanged for hydrogen. After being stirred at room temperature overnight the reaction was filtered through celite and concentrated. The crude bis(tert-Butyldimethylsilyloxy reaction product 8a was re-dissolved in a mixture of THF (165 mL) and methanol (250 mL) followed by the addition of a 37% aqueous hydrochloric acid solution (57 mL). After being stirred at room temperature for 2 h the reaction was carefully quenched with saturated bicarbonate solution and extracted with ethyl acetate (3×). The combined organic layers were washed with brine, dried over MgSO₄ and concentrated. The crude product was purified by automated column chromatography (silica gel, ethyl acetate/petroleum ether 2-100%) to yield 4.69 g (89%) of the required product 9a as a colourless foam.

Methyl 3α,7β-bis(tert-Butyldimethylsilyloxy)-25-homo-5β-cholan-25-oate (8a). ¹H NMR (500 MHz, CDCl₃) δ 3.69-3.62 (overlapping signals: m, 1H; 3.67, s, 3H), 3.52 (tt, 10.7, 4.7 Hz, 1H), 2.33-2.20 (m, 2H), 1.95 (dt, 12.5, 3.2 Hz, 1H), 1.84-1.60 (m, 6H); 1.58-1.15 (m, 14H), 1.15-1.03 (m, 3H), 1.03-0.91 (overlapping signals: m, 2H; 0.92, d, 3H; 0.92, s, 3H), 0.894 (s, 9H), 0.887 (s, 9H), 0.64 (s, 3H), 0.061 (s, 3H), 0.058 (s, 3H), 0.051 (s, 3H), 0.046 (s, 3H);

HRMS (ESI) m/z calcd for C₃₈H₇₂O₄Si₂Na⁺ 671.4861, found 671.4858.

Methyl 3α,7β-Dihydroxy-25-homo-5β-cholan-25-oate (9a). ¹H NMR (500 MHz, CDCl₃) δ 3.67 (s, 3H), 3.62-3.55 (m, 2H), 2.34-2.21 (m, 2H), 2.00 (dt, 12.6, 3.2 Hz, 1H), 1.91-1.83 (m, 1H), 1.83-1.75 (m, 3H), 1.75-1.64 (m, 3H), 1.63-1.54 (m, 2H), 1.54-1.35 (m, 10H), 1.33-1.19 (m, 4H), 1.14 (td, 12.9, 3.9 Hz, 1H), 1.11-0.98 (m, 3H), 0.95 (s, 3H), 0.94 (d, 6.7 Hz, 3H), 0.67 (s, 3H);

HRMS (ESI) m/z calcd for C₂₆H₄₄O₄Na⁺ 443.3132, found 443.3138.

### Tert-Butyl Ester

Using the same procedure as outlined for the preparation of methyl ester 8a, alkene 7b from Step E (21.7 g, 31.5 mmol) was dissolved in ethyl acetate (400 mL) and hydrogenated on 10% palladium on charcoal (867 mg) at room temperature overnight to afford 21.9 g (quant.) of 8b as a colourless resin.

¹H NMR (500 MHz, CDCl₃) δ 3.66 (ddd, 11.2, 8.6, 4.9 Hz, 1H), 3.52 (tt, 10.7, 4.7 Hz, 1H), 2.26-2.08 (m, 2H), 1.95 (dt, 12.5, 3.2 Hz, 1H), 1.86-1.71 (m, 4H), 1.70-1.59 (2H), 1.58-1.16 (m, 14H; 1.45, s, 9H), 1.15-0.99 (m, 4H), 0.99-0.91 (overlapping signals: m, 1H; 0.92, d, 3H; 0.92, s, 3H), 0.894 (s, 9H), 0.887 (s, 9H), 0.64 (s, 3H), 0.061 (s, 3H), 0.058 (s, 3H), 0.051 (s, 3H), 0.046 (s, 3H);

HRMS (ESI) m/z calcd for C₄₁H₇₈O₄Si₂Na⁺ 713.5331, found 713.5339.

A solution of silyl protected tert-butyl ester 8b (21.6 g, 31.3 mmol) in THF (100 mL) was treated with a 1 M solution of tetra-butyl ammonium fluoride in THF (144 mL, 144 mmol) at 50° C. for 72 h. After deprotection was complete (TLC analysis) water was added and the aqueous phase was extracted with ethyl acetate (3×). The organic phases were combined, washed with brine, dried over MgSO₄ and concentrated. The crude product was purified by automated column chromatography (silica gel, ethyl acetate/petroleum ether 5-80%) to give 13.9 g (96%) of the required product 9b as a colourless oil.

¹H NMR (500 MHz, CDCl₃) δ 3.63-3.55 (m, 2H), 2.24-2.11 (m, 2H), 2.00 (dt, 12.6, 3.2 Hz, 1H), 1.92-1.83 (m, 1H), 1.83-1.75 (m, 3H), 1.71-1.63 (m, 3H), 1.63-1.54 (m, 2H), 1.53-1.34 (m, 10H; 1.45, s, 9H), 1.33-1.19 (m, 4H), 1.14 (td, 12.9, 3.8 Hz, 1H), 1.11-0.98 (m, 3H), 0.95 (s, 3H), 0.94 (d, 6.6 Hz, 3H), 0.67 (s, 3H);

HRMS (ESI) m/z calcd for C₂₉H₅₀O₄Na⁺ 485.3601, found 485.3611.

### G. tert-Butyl 7p-hydroxy-3-oxo-25-homo-5β-cholan-25-oate (11)

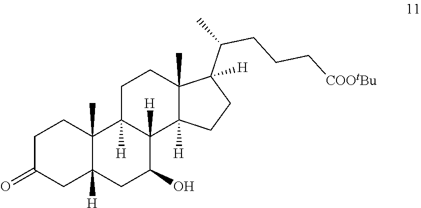

To a solution of 9b, the product of step F (13.9 g, 30.0 mmol) in dry dichloromethane (210 mL) was added (diacetoxyiodo)benzene (BAIB; 10.9 g, 33.8 mmol) followed by a catalytic amount of TEMPO (713 mg, 4.56 mmol) and the resulting reaction mixture was stirred at room temperature overnight. Subsequently, the reaction was quenched with saturated Na₂S₂O₃ solution and diluted with water. The organic layer was separated and the aqueous layer was extracted another three times with dichloromethane. The organic layers were combined, washed with brine, dried over MgSO₄ and concentrated. The residue was purified by automated column chromatography (silica gel, ethyl acetate/petroleum ether 2-80%) to afford 13.3 g (96%) of the required product 11 as a colourless foam.

¹H NMR (500 MHz, CDCl₃) δ 3.61 (ddd, 11.2, 8.8, 5.2 Hz, 1H), 2.52 (dd, 14.9, 14.1 Hz, 1H), 2.28 (td, 14.6, 5.4 Hz, 1H), 2.24-2.11 (m, 4H), 2.10-2.00 (m, 2H), 1.95-1.78 (m, 4H), 1.72-1.60 (m, 2H), 1.59-1.34 (m, 10H; 1.45, s, 9H), 1.34-1.16 (m, 3H), 1.14-1.03 (overlapping signals: m, 2H; 1.05, s, 3H), 0.95 (d, 6.6 Hz, 3H), 0.72 (s, 3H);

HRMS (ESI) m/z calcd for C₂₉H₄₈04Na⁺ 483.3445, found 483.3447.

The product may also be prepared from 9a by the same method.

### H. tert-Butyl 7p-hydroxy-3-oxa-4-oxo-4a,25-bishomo-5β-cholan-25-oate (side Product 14b) and methyl 7p-hydroxy-4-oxa-3-oxo-4a,25-bishomo-5β-cholan-25-oate (Required Product 15b)

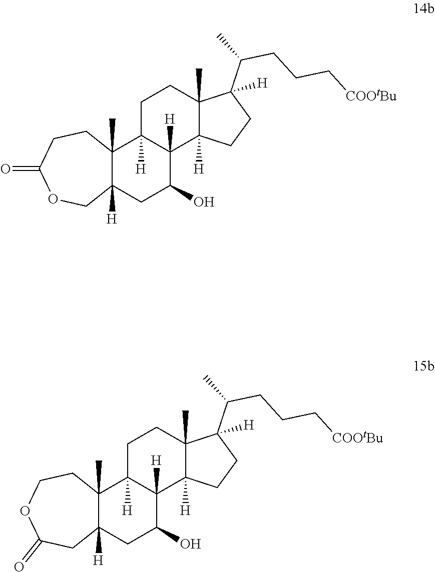

Following the same procedure as outlined for Example 1C, the product of Step G (11) (11.9 g, 25.8 mmol) in dry dichloromethane (200 mL) was treated with meta-chloroperbenzoic acid (mCPBA, 57-86%; 13.5 g, 54.8 mmol) at room temperature overnight to yield 11.1 g (90%) of the products 14b and 15b as a 1:1 mixture of isomers (colourless foam).

¹H NMR (500 MHz, CDCl₃, mixture of isomers) δ 4.48 (dd, 13.1, 9.6 Hz, 1H), 4.18 (dd, 13.3, 10.2 Hz, 1H), 4.05 (ddd, 13.2, 6.5, 1.0 Hz, 1H), 3.98 (ap d, 12.9 Hz, 1H), 3.48-3.40 (m, 1H), 3.33-3.25 (m, 1H), 3.03 (ap t, 12.9 Hz, 1H), 2.63 (ap dd, 14.5, 13.1, 1H), 2.42-2.35 (m, 2H), 2.24-2.11 (m, 4H), 2.08-2.01 (m, 3H), 1.98-1.84 (m, 7H), 1.84-1.75 (m, 2H), 1.74-1.61 (m, 4H), 1.58-1.31 (m, 20H; 1.44, s, 18H), 1.31-1.14 (m, 6H), 1.13-1.02 (overlapping signals: m, 4H; 1.05, s, 3H; 1.04, s, 3H), 0.939 (d, 6.6 Hz, 3H), 0.936 (d, 6.6 Hz, 3H), 0.70 (6H);

HRMS (ESI) m/z calcd for C₂₉H₄₈O₅Na⁺ (mixture of isomers) 499.3394, found 499.3398.

### I. tert-Butyl 4,7β-dihydroxy-25-homo-3,4-seco-5β-cholan-25-oate-3-amide (side Product 16b) and tert-butyl 2,7β-dihydroxy-25-homo-2,3-seco-5β-cholan-25-oate-3-amide (Required Product 17b)

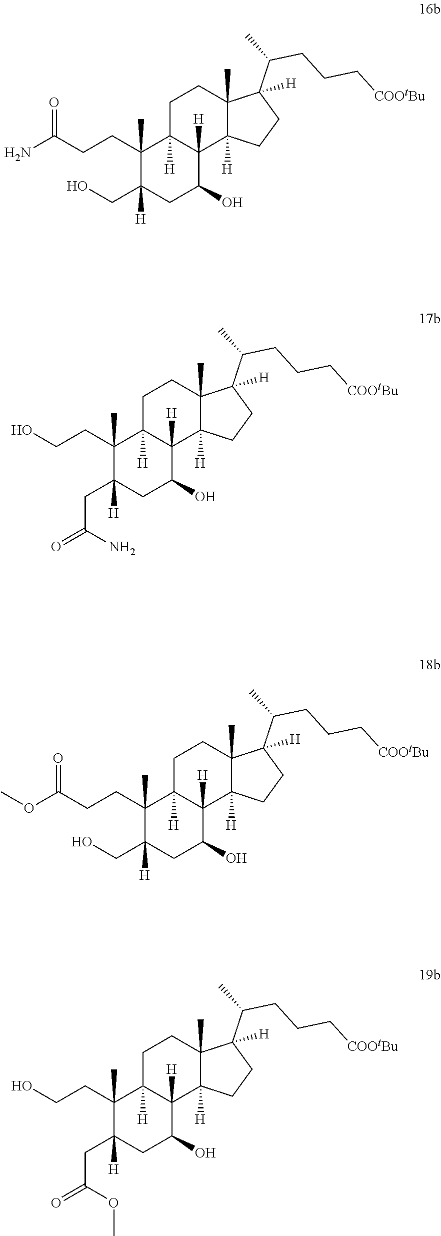

Deploying the same procedure as for Example 1D, the mixture of lactones 14b and 15b from Step H (10.9 g, 22.9 mmol) was dissolved in dry 7 N ammonia in methanol (155 mL) and heated at 92° C. in a 200 mL sealed tube with Teflon screw cap (the sealed tube was filled to 4/5 of its volume and a blast shield was added) overnight to give 8.52 g (76%) of a mixture of isomeric amides 16b and 17b as a colourless foam. The product ratio (16b:17b) was determined to be 1:1.3 by HPLC analysis (Phenomenex Luna C18(2) 5 μm 250×4.6 mm; Phenomenex Security Guard C18 4×3 mm; mobile phase: 45:55:0.05 water/acetonitrile/trifluoroacetic acid; flow rate: 1 mL/min; sample solvent: methanol; column temperature: 35° C.; injection volume: 25 μL; detection: refractive index). In addition, the isomeric methyl esters 18b (230 mg, 2%; colourless oil) and 19b (1.55 g, 13%, colourless foam) were isolated as by-products. An analytical sample of each of the four compounds was recovered for spectroscopic characterization.

tert-Butyl 4,7β-dihydroxy-25-homo-3,4-seco-5β-cholan-25-oate-3-amide (16b). ¹H NMR (500 MHz, CDCl₃) δ 6.00 (s_{br}, 1H, NH₂), 5.66 (s_{br}, 1H, NH₂), 3.76 (dd, 11.0, 5.1 Hz, 1H), 3.56 (dd, 11.0, 7.4 Hz, 1H), 3.49-3.41 (m, 1H), 2.41-2.29 (m, 1H), 2.22-2.08 (m, 3H), 2.03-1.95 (m, 2H), 1.91-1.74 (m, 3H), 1.74-1.68 (m, 1H), 1.68-1.54 (m, 3H), 1.53-1.33 (m, 6H; 1.44, s, 9H), 1.32-1.22 (m, 2H), 1.22-1.14 (m, 1H), 1.14-1.01 (m, 3H), 1.01-0.95 (overlapping signals: m, 1H; 0.98, s, 3H), 0.92 (d, 6.5 Hz, 3H), 0.68 (s, 3H); H,H—COSY (500 MHz, CDCl₃) δ 3.76 g 1.71, 3.56 g 1.71;

HRMS (ESI) m/z calcd for C₂₉H₅₁NO₅Na⁺ 516.3659, found 516.3669.

tert-Butyl 2,7β-dihydroxy-25-homo-2,3-seco-5β-cholan-24-oate-3-amide (17b). ¹H NMR (500 MHz, CDCl₃) δ 6.32 (s_{br}, 1H, NH₂), 6.00 (s_{br}, 1H, NH₂), 3.82-3.74 (m, 1H), 3.74-3.66 (m, 1H), 3.64-3.55 (m, 1H), 2.50-2.42 (m, 1H), 2.25-2.10 (m, 3H), 2.08-1.97 (m, 2H), 1.90-1.59 (m, 6H), 1.57-1.34 (m, 8H; 1.44, s, 9H), 1.33-1.23 (m, 3H), 1.23-1.10 (m, 2H), 1.10-1.00 (overlapping signals: m, 2H; 1.06, s, 3H), 1.00-0.90 (overlapping signals: m, 1H; 0.93, d, 6.4 Hz, 3H), 0.68 (s, 3H); H,H—COSY (500 MHz, CDCl₃) δ 3.78 g 1.72/1.41, 3.70 g 1.72/141;

HRMS (ESI) m/z calcd for C₂₉H₅₁NO₅H⁺494.3840, found 494.3846.

tert-Butyl 4,7β-dihydroxy-25-homo-3,4-seco-5β-cholan-25-oate-3-methyl ester (18b). ¹H NMR (500 MHz, CDCl₃) δ 3.72 (dd, 10.7, 4.6 Hz, 1H), 3.67 (s, 3H), 3.61-3.50 (overlapping signals: 3.58, dd, 10.8, 9.3 Hz, 1H; 3.54, ddd, 9.6, 11.5, 4.9 Hz, 1H), 2.37 (ddd, 15.5, 12.1, 4.5 Hz, 1H), 2.29 (dd, 12.0, 5.2 Hz, 1H), 2.22-2.12 (m, 2H), 2.07 (ddd, 13.4, 4.9, 2.9 Hz, 1H), 1.99 (dt, 12.8, 3.3 Hz, 1H), 1.95-1.71 (m, 3H), 1.71-1.61 (m, 2H), 1.61-1.53 (m, 2H), 1.53-1.33 (m, 8H; 1.44, s, 9H), 1.33-1.22 (m, 2H), 1.18 (ddd, 10.7, 12.4, 7.3 Hz, 1H), 1.15-1.04 (m, 3H), 1.04-0.96 (overlapping signals: m, 1H; 0.99, s, 3H), 0.92 (d, 6.6 Hz, 3H), 0.68 (s, 3H); H,H—COSY (500 MHz, CDCl₃) δ 3.72 g 1.67, 3.58 g 1.67;

HRMS (ESI) m/z calcd for C₃₀H₅₂O₆Na⁺ 531.3656, found 531.3670.

tert-Butyl 2,7β-dihydroxy-25-homo-2,3-seco-5β-cholan-25-oate-3-methyl ester (19b). ¹H NMR (500 MHz, CDCl₃) δ 3.77-3.68 (m, 2H), 3.68 (s, 3H), 3.58 (dt, 7.5, 9.3 Hz, 1H), 2.44 (dd, 14.9, 3.4 Hz, 1H), 2.33 (dd, 14.9, 11.4 Hz, 1H), 2.24-2.11 (m, 2H), 2.06-1.97 (m, 2H), 1.91-1.82 (m, 1H), 1.81-1.70 (m, 2H), 1.70-1.58 (m, 3H), 1.57-1.51 (m, 1H), 1.51-1.32 (m, 8H; 1.44, s, 9H), 1.32-1.24 (m, 2H), 1.20 (ddd, 10.7, 12.4, 7.3 Hz, 1H); 1.17-1.02 (overlapping signals: 1.13, td, 13.1, 4.0 Hz, 1H; m, 2H; 1.08, s, 3H), 1.00-0.91 (overlapping signals: m, 1H; 0.93, d, 6.6 Hz, 3H), 0.68 (s, 3H); H,H—COSY (500 MHz, CDCl₃) δ 3.72 (2H) g 1.74, 1.39;

HRMS (ESI) m/z calcd for C₃₀H₅₂O₆Na⁺ 531.3656, found 531.3662.

### J. tert-butyl 2-Amino-4,7β-dihydroxy-25-homo-3-nor-3,4-seco-5β-cholan-25-oate (Side Product 20b) and tert-butyl 4-Amino-2,7β-dihydroxy-25-homo-3-nor-3,4-seco-5β-cholan-25-oate (Required Product 21b)

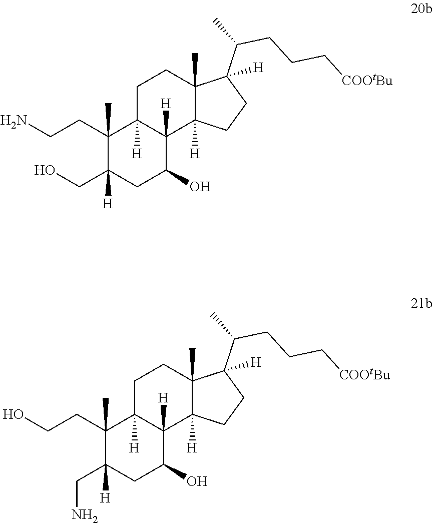

Employing the same procedure as for Example 1E, a mixture of the products of step I (16b and 17b) (7.85 g, 15.9 mmol) dissolved in a 3:1 mixture of acetonitrile and water (400 mL/132 mL; additionally 20 mL of ethyl acetate was added to help solubilize the starting material) was treated with (diacetoxyiodo)benzene (BAIB; 5.92 g, 18.4 mmol) at room temperature overnight to afford 5.55 g (75%) of a mixture of 20b and 21b as a colourless foam.

¹H NMR (500 MHz, CD₃OD; mixture of isomers) δ 3.71-3.57 (overlapping signals: 3.69, dd, 10.9, 4.6 Hz, 1H; m, 2H), 3.53 (dd, 10.9, 9.5 Hz, 1H), 3.46-3.37 (m, 2H), 2.84-2.75 (overlapping signals: 2.82, dd, 12.1, 4.8 Hz, 1H; m, 1H), 2.73-2.62 (overlapping signals: 2.70, td, 12.0, 4.7 Hz, 1H; 2.65, dd, 12.7, 10.9 Hz, 1H), 2.23-2.12 (m, 4H), 2.09-1.99 (m, 3H), 1.94-1.79 (m, 5H), 1.72-1.52 (m, 9H), 1.52-1.28 (m, 15H; 1.44, s, 18H), 1.28-1.11 (m, 6H), 1.11-0.98 (overlapping signals: m, 6H; 1.07, s, 3H; 1.06, s, 3H), 0.95 (d, 6.5 Hz, 6H), 0.72 (s, 6H);

HRMS (ESI) m/z calcd for C₂₈H₅₁NO₄H⁺ 466.3891, found 466.3894.

### K. tert-Butyl N-(benzyloxycarbonyl)-2-amino-4,7β-dihydroxy-25-homo-3-nor-3,4-seco-5β-cholan-25-oate (Side Product 22b) and tert-butyl N-(benzyloxycarbonyl)-4-amino-2,7β-dihydroxy-25-homo-3-nor-3,4-seco-5β-cholan-25-oate (Required Product 23b)

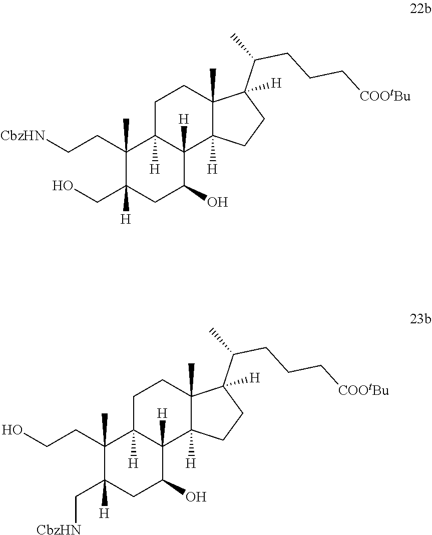

Following the same procedure as for Example 1F, the mixture of amines from Step J (20b and 21b) (5.48 g, 11.8 mmol) was dissolved in a 1:1 mixture of dichloromethane (80 mL) and water (80 mL) and treated with benzyl chloroformate (CbzCl 95%, 2.00 mL, 14.0 mmol) in the presence of sodium carbonate (6.44 g, 60.8 mmol) at 0° C. to yield 3.53 g (50%) of product 22b and 2.60 g (37%) of product 23b, both as colourless foams.

N-(Benzyloxycarbonyl)-2-amino-4,7β-dihydroxy-25-homo-3-nor-3,4-seco-5β-cholan-25-oate (22b) ¹H NMR (500 MHz, CDCl₃) δ 7.39-7.29 (m, 5H), 5.12-5.05 (m, 2H), 4.89-4.80 (m, 1H, NH), 3.92-3.83 (m, 1H), 3.60-3.52 (m, 1H), 3.51-3.44 (m, 1H), 3.44-3.34 (m, 1H), 3.14-3.04 (m, 1H), 2.34 (s_{br}, 1H, OH), 2.23-2.10 (m, 2H), 2.06-1.94 (m, 2H), 1.90-1.81 (m, 1H), 1.81-1.71 (m, 2H), 1.70-1.55 (m, 3H), 1.53-1.33 (m, 8H; 1.44, s, 9H), 1.32-1.21 (m, 2H), 1.20-1.13 (m, 1H), 1.13-0.94 (overlapping signals: m, 4H; 1.03, s, 3H), 0.92 (d, 6.6 Hz, 3H), 0.67 (s, 3H); H,H—COSY (500 MHz, CDCl₃) δ 3.88 g 1.74, 3.56 g 1.74;

HRMS (ESI) m/z calcd for C₃₆H₅₇NO₆H⁺ 600.4259, found 600.4269.

tert-Butyl N-(Benzyloxycarbonyl)-4-amino-2,7β-dihydroxy-25-homo-3-nor-3,4-seco-5β-cholan-25-oate (23b). ¹H NMR (500 MHz, CDCl₃) δ 7.41-7.29 (m, 5H), 5.09 (s, 2H), 4.98-4.90 (m, 1H, NH), 3.92-3.82 (m, 1H), 3.75-3.66 (m, 1H), 3.63-3.53 (m, 1H), 3.46-3.38 (m, 1H), 3.11 (ddd, 13.3, 11.5, 6.7 Hz, 1H), 2.23-2.11 (m, 2H), 2.02-1.95 (m, 1H), 1.90-1.80 (m, 3H), 1.80-1.70 (m, 2H), 1.70-1.61 (m, 2H), 1.61-1.33 (m, 9H; 1.44, s, 9H), 1.33-1.16 (m, 3H), 1.16-0.97 (overlapping signals: m, 4H, 1.04, s, 3H), 0.93 (d, 6.5 Hz, 3H), 0.67 (s, 3H); H,H—COSY (500 MHz, CDCl₃) δ 3.87 g 1.75, 1.49, 3.71 g 1.75, 1.49;

HRMS (ESI) m/z calcd for C₃₆H₅₇NO₆Na⁺ 622.4078, found 622.4082.

### L. tert-Butyl N-(benzyloxycarbonyl)-3-aza-7β-hydroxy-25-homo-5β-cholan-25-oate

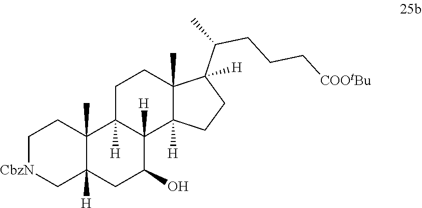

According to the protocol outlined for Example 1G, compound 23b of Step K (2.51 g, 4.18 mmol) dissolved in dry pyridine (80 mL) was treated with methanesulfonyl chloride (0.39 mL, 5.01 mmol) at 0° C. for 5 h, until only traces of starting material were detectable by TLC analysis. Then, saturated bicarbonate solution (4.5 mL) was added and the reaction mixture was stirred at 74° C. overnight. The crude product was purified by automated column chromatography (silica gel, ethyl acetate/petroleum ether 0-50%) to give 1.75 g (72%) of the product 25b as a colourless foam.

¹H NMR (500 MHz, CDCl₃) δ 7.39-7.28 (m, 5H), 5.18-5.06 (m, 2H), 3.98-3.75 (m, 2H), 3.57-3.45 (m, 1H), 3.08-2.90 (m, 1H), 2.90-2.73 (m, 1H), 2.23-2.11 (m, 2H), 2.02 (dt, 12.7, 3.01 Hz, 1H), 1.92-1.83 (m, 1H), 1.83-1.71 (m, 3H), 1.70-1.59 (m, 3H), 1.53-1.26 (m, 10H; 1.44, s, 9H), 1.26-1.02 (m, 5H), 0.99 (s, 3H), 0.93 (d, 6.6 Hz, 3H), 0.68 (s, 3H);

HRMS (ESI) m/z calcd for C₃₆H₅₅NO₅H⁺582.4153, found 582.4154.

### Example 17

### tert-Butyl 3-aza-7β-hydroxy-25-homo-5β-cholan-25-oate (27b)

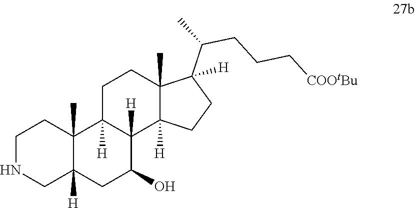

Using the same procedure as described for Example 2, the compound of Example 16 (25b) (865 mg, 1.48 mmol) dissolved in methanol (35 mL) was hydrogenated on 10% palladium on charcoal (93.8 mg) to yield 675 mg (quant.) of the product 27b as a colourless foam.

¹H NMR (500 MHz, CDCl₃) δ 3.51 (ddd, 11.6, 9.5, 5.1 Hz, 1H), 2.88 (ap t, 12.5 Hz, 1H), 2.77-2.63 (m, 3H), 2.24-2.11 (m, 2H), 2.04-1.98 (m, 1H), 1.92-1.73 (m, 6H), 1.71-1.62 (m, 1H), 1.62-1.54 (m, 3H), 1.52-1.33 (m, 6H; 1.44, s, 9H), 1.33-1.22 (m, 3H), 1.22-1.02 (m, 4H), 0.99 (s, 3H), 0.94 (d, 6.6 Hz, 3H), 0.68 (s, 3H);

HRMS (ESI) m/z calcd for C₂₈H₄₉NO₃H⁺ 448.3785, found 448.3787.

### Example 18

### 3-Aza-7β-hydroxy-25-homo-5β-cholan-25-oic acid hydrochloride (28b)

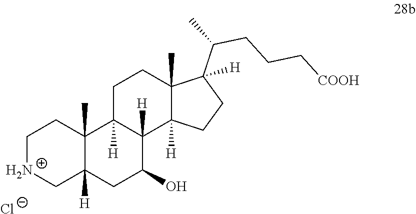

Applying the same protocol as for Example 3, tert-butyl ester 27b from Example 17 (151 mg, 0.337 mmol) dissolved in dry dichloromethane (4 mL) was deprotected with trifluoroacetic acid (TFA; 3 mL) at 0° C. to yield 113 mg (78%) of 28b as a colourless foam.

¹H NMR (500 MHz, CD₃OD) δ 3.41-3.34 (m, 1H), 3.24 (apt, 13.0 Hz, 1H), 3.15-3.05 (m, 2H), 3.01 (td, 13.5, 2.3 Hz, 1H), 2.31-2.18 (m, 2H), 2.09-1.99 (m, 2H), 1.95-1.81 (m, 4H), 1.74-1.65 (m, 1H), 1.65-1.60 (m, 1H), 1.60-1.36 (m, 9H), 1.35-1.21 (m, 3H), 1.16-1.05 (overlapping signals: m, 2H; 1.09, s, 3H), 0.97 (d, 6.6 Hz, 3H), 0.73 (s, 3H);

¹³C NMR (125 MHz, CD₃OD) δ 177.78, 71.06, 56.95, 56.61, 45.03, 44.75, 44.37, 42.08, 41.19, 40.80, 40.00, 36.87, 36.64, 35.41, 35.17, 34.26, 33.56, 29.70, 27.91, 23.48, 22.75, 22.55, 19.27, 12.61;

HRMS (ESI) m/z calcd for C₂₄H₄₁NO₃H⁺ 392.3159, found 392.3162.

### Example 19

### tert-Butyl N-methyl-3-aza-7β-hydroxy-25-homo-5β-cholan-25-oate (29b)

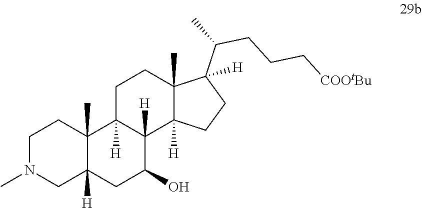

Using the same procedure as for Example 4, compound 25b of Example 16 (70.1 mg, 0.121 mmol) dissolved in methanol (5 mL) was treated with formalin (37% in water, 0.1 mL, 1.34 mmol) and stirred under an atmosphere of hydrogen in the presence of 10% palladium on charcoal (20.3 mg) to give 52.2 mg (94%) of 29b as a colourless oil.

¹H NMR (500 MHz, CDCl₃) δ 3.53 (ddd, 10.8, 9.3, 5.0, 1H), 2.56-2.46 (m, 2H), 2.25 (s, 3H), 2.23-2.11 (m, 3H), 2.03-1.94 (m, 2H), 1.91-1.83 (m, 1H), 1.83-1.73 (m, 4H), 1.71-1.56 (m, 2H), 1.52-1.33 (m, 8H; 1.44, s, 9H), 1.33-1.20 (m, 3H), 1.15 (td, 13.0, 3.9 Hz, 1H), 1.12-1.02 (m, 2H), 0.99 (s, 3H), 0.94 (d, 6.6 Hz, 3H), 0.68 (s, 3H);

HRMS (ESI) m/z calcd for C₂₉H₅₁NO₃H⁺ 462.3942, found 462.3953.

### Example 20

### N-Methyl-3-aza-7β-hydroxy-25-homo-5β-cholan-25-oic acid hydrotrifluoroacetate (31b)

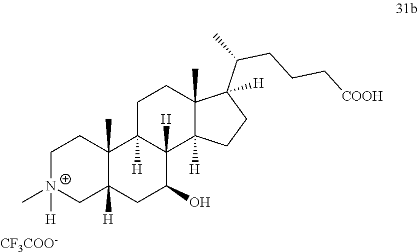

Employing the same reaction protocol as for Example 5, the tert-butyl ester 29b of Example 19 (52.2 mg, 0.113 mmol) dissolved in dry dichloromethane (5 mL) was treated with trifluoroacetic acid (4 mL) at 0° C. to afford 48.8 mg (83%) of the product 31 b as a colourless foam.

¹H NMR (500 MHz, CD₃OD) δ 3.38 (ddd, 11.4, 9.7, 5.1, 1H), 3.30-3.17 (m, 3H), 3.05-2.97 (m, 1H), 2.85 (s, 3H), 2.31-2.18 (m, 2H), 2.10-2.02 (m, 2H), 1.95-1.80 (m, 4H), 1.75-1.66 (m, 1H), 1.66-1.54 (m, 2H), 1.54-1.37 (m, 8H), 1.327-1.22 (m, 3H), 1.16-1.04 (overlapping signals: m, 2H; 1.08, s, 3H), 0.97 (d, 6.6 Hz, 3H), 0.73 (s, 3H);

¹⁹F NMR (470 MHz, CD₃OD) δ −76.92;

¹³C NMR (125 MHz, CD₃OD) δ 177.71, 70.95, 56.81, 56.63, 55.40, 51.06, 44.74, 44.36, 43.74, 42.94, 41.12, 39.79, 36.86, 36.63, 35.39, 35.06, 34.40, 33.47, 29.70, 27.91, 23.06, 22.75, 22.55, 19.27, 12.60;

HRMS (ESI) m/z calcd for C₂₅H₄₃NO₃H⁺ 406.3316, found 406.3315.

### Example 21

### tert-Butyl N-glycolyl-3-aza-7β-hydroxy-25-homo-5β-cholan-25-oate (34b)

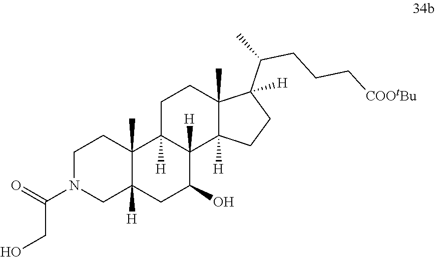

Using the same procedure as for Example 9, compound 27b of Example 17 (102 mg, 0.228 mmol) was dissolved in dry dichloromethane (2.5 mL) and sequentially treated with glycolic acid (27.7 mg, 0.364 mmol), 1-hydroxybenzotriazole (HOBt; 33.8 mg, 0.250 mmol), N,N-diisopropylethylamine (0.087 mL, 0.499 mmol) and N-(3-dimethylaminopropyl)-N′-ethylcarbodiimide hydrochloride (EDCI; 60.8 mg, 0.317 mmol) to yield 107 mg (93%) of 34b as a colourless foam.

¹H NMR (500 MHz, CDCl₃; mixture of rotamers) δ 4.34-4.24 (m, 1H), 4.19-4.06 (m, 2H), 3.66 (s_{br}, 1H, OH), 3.57-3.47 (m, 1H), 3.25-3.15 (m, 1H), 3.11 (dd, 13.5, 4.2 Hz, 0.5H), 3.01 (td, 13.4, 1.9 Hz, 0.5H), 2.90 (ap t, 13.2 Hz, 0.5H), 2.77-2.68 (m, 0.5H), 2.24-2.11 (m, 2H), 2.07-2.00 (m, 1H), 1.93-1.75 (m, 4H), 1.76-1.60 (m, 3H), 1.59-1.34 (m, 9H; 1.44, s, 9H), 1.34-1.05 (6H), 1.02 (s, 3H), 0.94 (d, 6.3 Hz, 3H), 0.70 (s, 3H);

HRMS (ESI) m/z calcd for C₃₀H₅₁NO₅Na⁺ 528.3659, found 528.3658.

### Example 22

### N-Glycolyl-3-aza-7β-hydroxy-25-homo-5β-cholan-25-oic acid (40b)

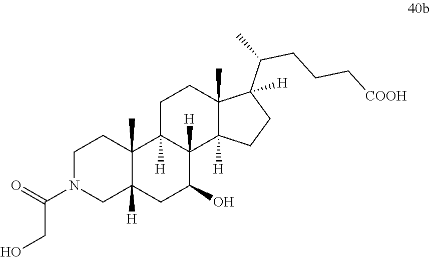

Employing the same protocol as for Example 10, compound 34b of Example 21 (93.8 mg, 0.186 mmol) dissolved in dry dichloromethane (5 mL) was treated with trifluoroacetic acid (4 mL) at 0° C. for 2.5 h to afford 45.1 mg (54%) of the product 40b as a colourless foam.

¹H NMR (500 MHz, CD₃OD; mixture of rotamers) δ 4.25-4.12 (m, 3H), 3.48-3.38 (m, 1.5H), 3.38-3.27 (m, 1H), 3.09 (td, 13.4, 1.6 Hz, 0.5H), 2.96 (apt, 13.1 Hz, 0.5H), 2.80-2.71 (m, 0.5H), 2.31-2.17 (m, 2H), 2.08-2.02 (m, 1H), 1.94-1.80 (m, 4H), 1.74-1.54 (m, 4H), 1.54-1.35 (m, 7H), 1.33-1.05 (m, 6H), 1.03 (s, 3H), 0.97 (d, 6.5 Hz, 3H), 0.73 (s, 3H);

¹³C NMR (125 MHz, CD₃OD; mixture of rotamers) δ 177.75, 171.78, 71.61, 71.57, 61.24, 61.17, 57.27, 57.25, 56.66, 45.84, 44.99, 44.77, 44.38, 43.87, 41.40, 40.82, 40.22, 38.81, 37.07, 36.90, 36.66, 36.47, 36.07, 35.93, 35.42, 35.06, 29.72, 27.95, 23.78, 23.73, 22.79, 22.64, 22.60, 19.29, 12.67;

HRMS (ESI) m/z calcd for C₂₆H₄₃NO₅Na⁺ 472.3033, found 472.3028.

### Example 23

### N-[(2S)-2-Amino-3-methylbutanoyl]-3-aza-7β-hydroxy-25-homo-5β-cholan-25-oic acid hydrotrifluoroacetate (41b)

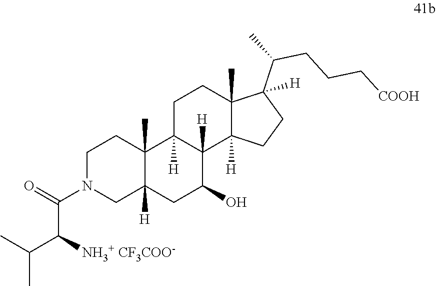

### A. tert-Butyl N-{(2S)-2-[(tert-butoxycarbonyl)amino]-3-methylbutanoyl}-3-aza-7β-hydroxy-25-homo-5β-cholan-25-oate (35b)

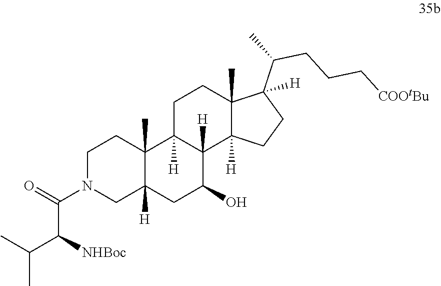

Following the same procedure as for Example 5, compound 27b of Example 17 (100 mg, 0.223 mmol) was dissolved in dry dichloromethane (2.5 mL) and reacted with Boc-L-valine (74.6 mg, 0.343 mmol), 1-hydroxybenzotriazole (33.4 mg, 0.247 mmol), N,N-diisopropylethylamine (0.086 mL, 0.494 mmol) and N-(3-dimethylaminopropyl)-N′-ethylcarbodiimide hydrochloride (56.3 mg, 0.294 mmol) to give 154 mg (quant.) of the product 35b as a colourless oil.

¹H NMR (500 MHz, CDCl₃; mixture of rotamers) δ 5.68-5.57 (m, 1H, NH), 4.52-4.41 (m, 1H), 4.38-4.31 (m, 0.5H), 4.27 (dd, 13.6, 3.6 Hz, 0.5H), 3.78-3.67 (m, 1H), 3.60-3.49 (m, 1H), 3.28 (ap t, 13.0 Hz, 0.5H), 3.23-3.14 (m, 0.5H), 2.87 (ap t, 13.3 Hz, 0.5H), 2.67 (td, 13.3, 1.9 Hz, 0.5H), 2.25-2.12 (m, 2H), 2.07-2.01 (m, 1H), 1.97-1.75 (m, 5H), 1.75-1.54 (m, 3H), 1.53-1.34 (m, 8H; 1.45, s, 9H; 1.429, s, 4.5H; 1.425, s, 4.5H), 1.34-1.12 (m, 4H), 1.12-1.04 (m, 2H), 1.03 (s, 1.5H), 1.00 (s, 1.5H), 0.97-0.91 (overlapping signals: m, 1.5H; 0.96, d, 6.8 Hz, 1.5H; 0.94, d, 6.7 Hz, 3H; 0.93, d, 6.8 Hz, 1.5H); 0.89 (d, 6.8 Hz, 1.5H), 0.695 (s, 1.5H), 0.692 (s, 1.5H);

HRMS (ESI) m/z calcd for C₃₈H₆₆N₂O₆Na⁺669.4813, found 669.4817.

### B. N-[(2S)-2-Amino-3-methylbutanoyl]-3-aza-7β-hydroxy-25-homo-5β-cholan-25-oic acid hydrotrifluoroacetate (41b)

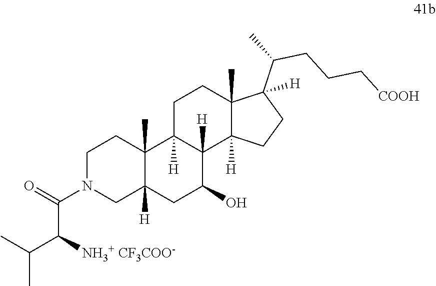

Following the same method as or Example 12, step B, compound 35b of step A (137 mg, 0.212 mmol) was dissolved in dry dichloromethane (5 mL) and treated with trifluoroacetic acid (4 mL) to yield 80.1 mg (63%) of the product 41b as a colourless foam.

¹H NMR (500 MHz, CD₃OD; mixture of rotamers) δ 4.35 (d, 3.9 Hz, 0.5H), 4.32-4.26 (m, 0.5H), 4.24 (d, 5.0 Hz, 0.5H), 4.21-4.16 (m, 0.5H), 3.64-3.59 (m, 0.5H), 3.59-3.53 (m, 0.5H), 3.49-3.38 (m, 1.5H), 3.20 (td, 13.5, 1.6 Hz, 0.5H), 2.99 (ap t, 13.3 Hz, 0.5H), 2.79 (td, 13.4, 1.9 Hz, 0.5H), 2.31-2.12 (m, 3H), 2.09-2.03 (m, 1H), 1.97-1.80 (m, 4H), 1.74-1.58 (m, 3.5H), 1.58-1.35 (m, 7.5H), 1.34-1.19 (m, 3.5H), 1.19-1.07 (overlapping signals: m, 2.5H; 1.10, d, 7.3 Hz, 1.5H; 1.09, d, 7.25 Hz, 1.5H), 1.05 (s, 1.5H), 1.03 (s, 1.5H), 1.01 (d, 7.0 Hz, 1.5H), 0.99 (d, 7.0 Hz, 1.5H), 0.97 (d, 6.5 Hz, 3H), 0.74 (s, 3H);

¹⁹F NMR (470 MHz, CD₃OD) δ −76.97;

¹³C NMR (125 MHz, CD₃OD; mixture of rotamers) δ 177.74, 168.04, 167.88, 71.59, 71.54, 57.31, 57.12, 56.62, 56.34, 48.02, 45.75, 44.77, 44.45, 44.38, 44.18, 42.36, 41.41, 41.35, 40.47, 40.17, 39.21, 37.22, 36.89, 36.65, 36.47, 36.04, 35.79, 35.39, 35.19, 34.96, 30.95, 29.72, 27.96, 23.79, 23.68, 22.75, 22.64, 22.58, 19.29, 19.10, 17.04, 16.83, 12.65;

HRMS (ESI) m/z calcd for C₂₉H₅₀N₂O₄H⁺ 491.3843, found 491.3856.

### Example 24

### N-[(2S,3S)-2-Amino-3-methylpentanoyl]-3-aza-7β-hydroxy-25-homo-5β-cholan-25-oic acid hydrotrifluoroacetate (42b)

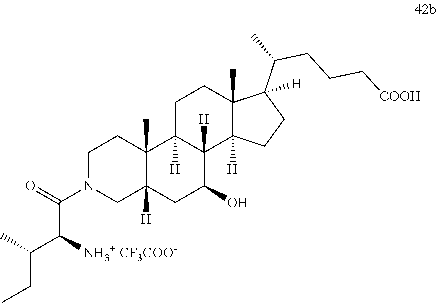

### A. tert-Butyl N-{(2S,3S)-2-[(tert-butoxycarbonyl)amino]-3-methylpentanoyl}-3-aza-7p-hydroxy-25-homo-5β-cholan-25-oate (36b)

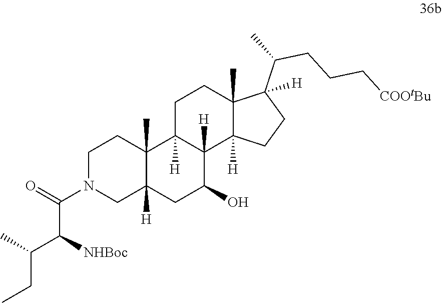

Employing the same protocol as for Example 9, compound 27b of Example 17 (61.5 mg, 0.137 mmol) was dissolved in dry dichloromethane (2 mL) and reacted with Boc-L-isoleucine (48.3 mg, 0.209 mmol), 1-hydroxybenzotriazole (20.9 mg, 0.155 mmol), N,N-diisopropylethylamine (0.053 mL, 0.304 mmol) and N-(3-dimethylaminopropyl)-N′-ethylcarbodiimide hydrochloride (34.8 mg, 0.182 mmol) to afford 88.1 mg (97%) of the product 36b as a colourless oil.

¹H NMR (500 MHz, CDCl₃; mixture of rotamers) δ 5.33-5.25 (m, 1H, NH), 4.50-4.42 (m, 1H), 4.39-4.33 (m, 0.5H), 4.28 (dd, 13.5, 3.5 Hz, 0.5H), 3.74-3.60 (m, 1H), 3.56-3.46 (m, 1H), 3.25 (ap t, 13.0 Hz, 0.5H), 3.16-3.08 (m, 0.5H), 2.81 (ap t, 13.3 Hz, 0.5H), 2.61 (td, 13.3, 1.9 Hz, 0.5H), 2.24-2.11 (m, 2H), 2.04-2.00 (m, 1H), 1.93-1.75 (m, 4H), 1.73-1.59 (m, 3.5H), 1.59-1.32 (m, 10.5H; 1.44; s, 9H; 1.425, s, 4.5H; 1.423, s, 4.5H), 1.32-1.13 (m, 4H), 1.13-1.04 (m, 3H), 1.02 (s, 1.5H), 0.99 (s, 1.5H), 0.96-0.85 (overlapping signals: 0.94, d, 6.5 Hz, 3H; 0.93, d, 7.2 Hz, 1.5H; 0.92, d, 7.2 Hz, 1.5H; 0.88, t, 7.3 Hz, 1.5H; 0.87, t, 7.3 Hz, 1.5H), 0.694 (s, 1.5H), 0.692 (s, 1.5H);

HRMS (ESI) m/z calcd for C₃₉H₆₈N₂O₆H⁺661.5150, found 661.5152.

### B. N-[(2S,3S)-2-Amino-3-methylpentanoyl]-3-aza-7β-hydroxy-25-homo-5β-cholan-25-oic acid hydrotrifluoroacetate (42b)

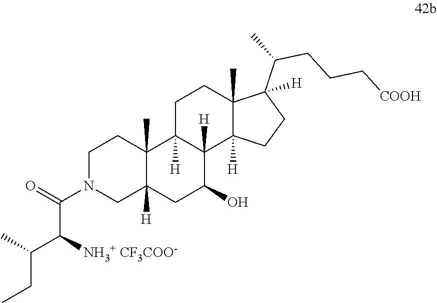

According to the method of Example 11, step B, compound 36b of step A (88.1 mg, 0.133 mmol) was dissolved in dry dichloromethane (4 mL) and treated with trifluoroacetic acid (3 mL) to give 38.8 mg (47%) of the product 42b as a colourless foam.

¹H NMR (500 MHz, CD₃OD; mixture of rotamers) δ 4.36 (d, 4.8 Hz, 0.5H), 4.32-4.24 (overlapping signals: m, 0.5H; 4.25, d, 4.9 Hz, 0.5H), 4.18 (dd, 13.7, 3.6 Hz, 0.5H), 3.63-3.52 (overlapping signals: m, 0.5H; 3.55, dd, 13.5, 3.0 Hz, 0.5H), 3.50-3.38 (m, 1.5H), 3.25-3.17 (m, 0.5H), 2.99 (ap t, 13.3 Hz, 0.5H), 2.79 (td, 13.2, 1.6 Hz, 0.5H), 2.31-2.18 (m, 2H), 2.09-2.03 (m, 1H), 1.99-1.81 (m, 5H), 1.74-1.57 (m, 3.5H), 1.57-1.35 (m, 8.5H), 1.34-1.15 (m, 5H), 1.15-1.04 (overlapping signals: m, 2H; 1.08, d, 7.0 Hz, 1.5H; 1.06, d, 7.0 Hz, 1.5H; 1.05, s, 1.5H), 1.03 (s, 1.5H), 1.00-0.94 (m, 6H), 0.74 (s, 3H);

¹⁹F NMR (470 MHz, CD₃OD) δ −76.96;

¹³C NMR (125 MHz, CD₃OD; mixture of rotamers) δ 177.73, 168.03, 167.87, 71.59, 71.53, 57.30, 57.11, 56.67, 56.62, 56.33, 56.00, 48.13, 45.76, 44.77, 44.52, 44.44, 44.38, 44.24, 42.40, 41.41, 41.34, 40.50, 40.16, 39.25, 37.71, 37.67, 37.33, 36.88, 36.65, 36.45, 36.04, 35.80, 35.39, 35.20, 34.97, 29.73, 27.96, 24.69, 24.51, 23.78, 23.74, 22.75, 22.64, 22.58, 19.28, 15.80, 15.46, 12.64, 12.06, 11.86;

HRMS (ESI) m/z calcd for C₃₀H₅₂N₂O₄H⁺505.4000, found 505.4010.

### Example 25

### N-[(2S)-2-Amino-4-methylpentanoyl]-3-aza-7β-hydroxy-25-homo-5β-cholan-25-oic acid hydrotrifluoroacetate (43b)

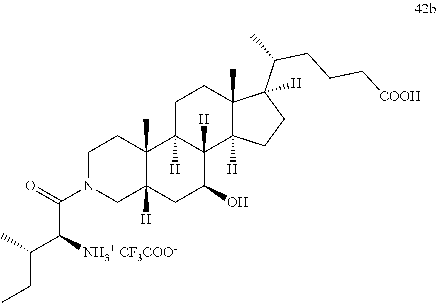

### A. tert-Butyl N-{(2S)-2-[(tert-butoxycarbonyl)amino]-4-methylpentanoyl}-3-aza-7β-hydroxy-25-homo-5β-cholan-25-oate (37b)

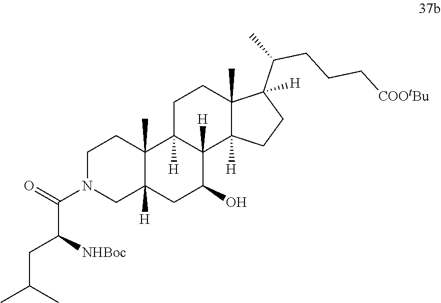

Using the same procedure as for Example 9, compound 27b of Example 17 (60.4 mg, 0.135 mmol) was dissolved in dry dichloromethane (2 mL) and reacted with Boc-L-isoleucine (46.9 mg, 0.203 mmol), 1-hydroxybenzotriazole (20.5 mg, 0.152 mmol), N,N-diisopropylethylamine (0.052 mL, 0.299 mmol) and N-(3-dimethylaminopropyl)-N′-ethylcarbodiimide hydrochloride (33.9 mg, 0.177 mmol) to give 71.1 mg (80%) of the product 37b as a colourless oil.

¹H NMR (500 MHz, CDCl₃; mixture of rotamers) δ 5.33 (s, 0.5H, NH), 5.32 (s, 0.5H, NH), 4.68-4.60 (m, 1H), 4.36-4.29 (m, 0.5H), 4.25 (dd, 13.6, 3.8 Hz, 0.5H), 3.65-3.56 (m, 1H), 3.56-3.46 (m, 1H), 3.23 (ap t, 13.1 Hz, 0.5H), 3.17-3.08 (m, 0.5H), 2.81 (ap t, 13.2 Hz, 0.5H), 2.59 (td, 13.3, 1.9 Hz, 0.5H), 2.24-2.11 (m, 2H), 2.06-2.00 (m, 1H), 1.93-1.75 (m, 4H), 1.75-1.54 (m, 4H), 1.54-1.13 (m, 15H; 1.44, s, 9H; 1.429, s, 4.5H; 1.425, s, 4.5H); 1.13-1.04 (m, 2H); 1.02 (s, 1.5H), 1.01-0.97 (overlapping signals: 1.00, s, 1.5H; m, 3H), 0.96-0.90 (overlapping signals: 0.94, d, 6.6 Hz, 3H; m, 3H), 0.69 (s, 3H);

HRMS (ESI) m/z calcd for C₃₉H₆₈N₂O₆Na⁺ 683.4970, found 683.4970.

### B. N-[(2S)-2-Amino-4-methylpentanoyl]-3-aza-7β-hydroxy-25-homo-5β-cholan-25-oic acid hydrotrifluoroacetate (43b)

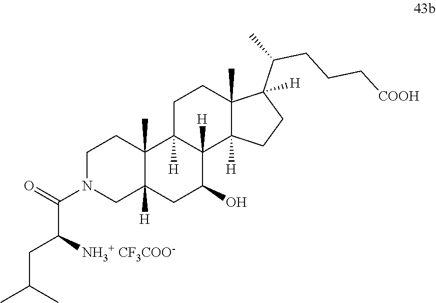

Employing the same reaction conditions as for Example 11, step B, compound 37b of step A (71.1 mg, 0.108 mmol) was dissolved in dry dichloromethane (3 mL) was treated with trifluoroacetic acid (2 mL) at 0° C. for 2.5 h to yield 50.8 mg (76%) of the product 43b as a colourless foam.

¹H NMR (500 MHz, CD₃OD; mixture of rotamers) δ 4.43 (dd, 9.3, 4.0 Hz, 0.5H), 4.35 (dd, 9.1, 3.7 Hz, 0.5H), 4.28-4.22 (m, 0.5H), 4.17 (dd, 13.6, 3.8 Hz, 0.5H), 3.52-3.38 (m, 2.5H), 3.27-3.19 (m, 0.5H), 2.99 (ap t, 13.2 Hz, 0.5H), 2.77 (td, 13.3, 1.5 Hz, 0.5H), 2.31-2.18 (m, 2H), 2.10-2.02 (m, 1H), 1.99-1.93 (m, 0.5H), 1.93-1.81 (m, 3.5H), 1.80-1.55 (m, 7H), 1.55-1.36 (m, 7H), 1.35-1.16 (m, 4H), 1.16-1.07 (m, 2H), 1.06 (s, 1.5H), 1.05-0.99 (overlapping signals: 1.04, s, 1.5H; m, 6H), 0.97 (d, 6.5 Hz, 3H), 0.74 (s, 3H);

¹⁹F NMR (470 MHz, CD₃OD) δ −76.90;

¹³C NMR (125 MHz, CD₃OD; mixture of rotamers) δ 177.73, 168.82, 168.74, 71.56, 71.53, 57.29, 57.12, 56.68, 56.64, 50.45, 47.65, 45.43, 44.77, 44.41, 44.39, 44.25, 44.11, 42.21, 41.55, 41.40, 41.34, 41.02, 40.46, 40.18, 39.18, 37.06, 36.88, 36.65, 36.42, 36.00, 35.97, 35.39, 35.12, 35.02, 29.72, 27.96, 25.51, 25.45, 23.77, 23.64, 23.60, 23.58, 22.75, 22.63, 22.58, 21.83, 21.72, 19.28, 12.66;

HRMS (ESI) m/z calcd for C₃₀H₅₂N₂O₄H⁺ 505.4000, found 505.4011.

### Example 26

### N-[(2S)-2-Amino-3-(1H-indol-3-yl)propanoyl]-3-aza-7β-hydroxy-25-homo-5β-cholan-25-oic acid hydrotrifluoroacetate (44b)

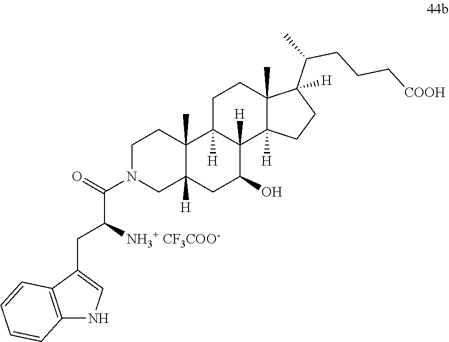

### A. tert-Butyl N-{(2S)-2-[(tert-butoxycarbonyl)amino]-3-[1H-indol-3-yl]propanoyl}-3-aza-7β-hydroxy-25-homo-5β-cholan-25-oate (38b)

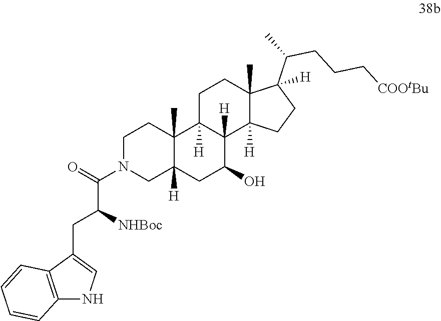

Applying the same method as for Example 9, compound 27b of Example 17 (49.8 mg, 0.111 mmol) was dissolved in dry dichloromethane (2 mL) and treated with Na-Boc-L-tryptophan (51.3 mg, 0.169 mmol), 1-hydroxybenzotriazole (16.6 mg, 0.123 mmol), N,N-diisopropylethylamine (0.043 mL, 0.247 mmol) and N-(3-dimethylaminopropyl)-N′-ethylcarbodiimide hydrochloride (30.4 mg, 0.159 mmol) to give 75.5 mg (93%) of the product 38b as a colourless oil.

¹H NMR (500 MHz, CDCl₃; mixture of rotamers) δ 8.32 (s, 0.5H, NH), 8.22 (s, 0.5H, NH), 7.68-7.62 (m, 0.5H), 7.59-7.55 (m, 0.5H), 7.36-7.29 (m, 1H), 7.20-7.07 (m, 2H), 7.06-7.01 (m, 1H), 5.69 (d, 8.4 Hz, 0.5H, NH), 5.52 (d, 8.7 Hz, 0.5H, NH), 4.99-4.92 (m, 0.5H), 4.92-4.84 (m, 0.5H), 4.15-4.05 (m, 1H), 3.43-3.33 (m, 0.5H), 3.29-3.19 (m, 1H), 3.18-3.12 (m, 1H), 3.12-2.99 (overlapping signals: 3.08, dd, 14.0, 9.5 Hz, 0.5H; 3.03, dd, 13.4, 3.5 Hz, 0.5H), 2.84-2.74 (m, 1H), 2.60 (ap t, 13.2 Hz, 0.5H), 2.27-2.09 (m, 2.5H), 1.99-0.98 (m, 31H; 1.46, s, 4.5H, 1.43, s, 4.5H), 0.95 (d, 6.6 Hz, 1.5H), 0.92-0.80 (overlapping signals: 0.90, d, 6.6 Hz, 1.5H; m, 0.5H; 0.83, s, 1.5H), 0.80-0.72 (m, 0.5H), 0.64-0.59 (overlapping singlets: 0.62, 1.5H; 0.61, 3H), −0.34 (td, 13.2, 2.8 Hz, 0.5H);

### B. N-[(2S)-2-Amino-3-(1H-indol-3-yl)propanoyl]-3-aza-7β-hydroxy-25-homo-5β-cholan-25-oic acid hydrotrifluoroacetate (44b)

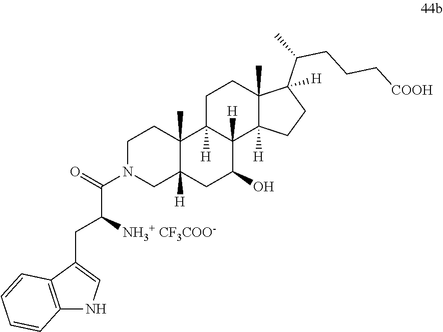

Deploying the same reaction conditions as for Example 11, step B compound 38b of step A (75.5 mg, 0.103 mmol) dissolved in dry dichloromethane (5 mL) was treated with trifluoroacetic acid (4 mL) to afford 47.6 mg (67%) of the product 44b as a colourless foam.

¹H NMR (500 MHz, CD₃OD; mixture of rotamers) δ 7.55-7.51 (m, 0.5H), 7.46-7.36 (m, 1.5H), 7.22 (s, 0.5H), 7.20 (s, 0.5H), 7.19-7.07 (m, 1.5H), 7.06-7.02 (m, 0.5H), 4.65-4.58 (m, 1H), 4.03-3.96 (m, 1H), 3.36-3.20 (overlapping signals: m, 2H; 3.23, dd, 13.8, 10.5 Hz, 0.5H), 3.14-3.08 (m, 0.5H), 2.94 (dd, 13.5, 3.7 Hz, 0.5H), 2.84 (td, 13.2, 1.9 Hz, 0.5H), 2.77-1.65 (m, 1H), 2.34-2.15 (m, 2.5H), 2.00-1.63 (m, 4H), 1.63-1.01 (m, 16.5H), 1.01-0.93 (overlapping signals: m, 0.5H; 1.00, d, 6.5 Hz, 1.5H), 0.92 (d, 6.6 Hz, 1.5H), 0.85 (s, 1.5H), 0.70-0.63 (overlapping signals: m, 1H; 0.66, s, 1.5H; 0.65, s, 1.5H), 0.60 (s, 1.5H), −0.52 (td, 13.7, 4.1 Hz, 0.5H);

¹⁹F NMR (470 MHz, CD₃OD) δ −76.92;

¹³C NMR (125 MHz, CD₃OD; mixture of rotamers) δ 177.78, 177.73, 169.38, 168.66,138.14, 138.04, 128.82, 128.73, 125.76, 125.50, 123.32, 123.05, 120.52, 120.44, 119.31, 119.12, 112.98, 112.90, 108.64, 108.31, 71.44, 71.08, 57.23, 56.88, 56.78, 56.61, 52.13, 51.27, 46.43, 44.73, 44.69, 44.22, 44.09, 43.97, 43.86, 43.12, 42.59, 41.32, 41.21, 40.17, 40.13, 38.68, 36.91, 36.83, 36.70, 36.61, 35.94, 35.85, 35.56, 35.42, 35.36, 35.21, 34.10, 29.74, 29.65, 29.26, 29.16, 27.87, 27.77, 23.22, 23.04, 22.79, 22.72, 22.44, 22.34, 19.33, 19.24, 12.60;

HRMS (ESI) m/z calcd for C₃₅H₅₁N₃O₄H⁺ 578.3952, found 578.3958.

### Example 27

### Conjugates of of 3-aza-7β-hydroxy-5β-cholan-24-oic acid (Compound 28a of Example 3) and 3-aza-7β-hydroxy-25-homo-5β-cholan-24-oic acid (Compound 28b of Example 18)

### I. Preparation of N-(benzyloxycarbony)-3-aza-7β-hydroxy-5β-cholan-24-oic acid (50a)

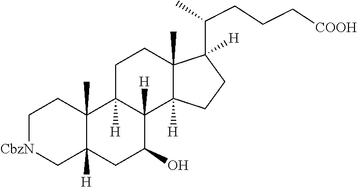

To a solution of tert-butyl N-(benzyloxycarbonyl)-3-aza-7β-hydroxy-5β-cholan-25-oate (Compound 25a of Example 1) (2.6 g, 4.5 mmol) in anhydrous DCM (67 mL, 26 vol) was added trifluoroacetic acid (TFA; 52 mL, 20 vol) at 0° C. After being stirred for 3.5 h at 0° C. the reaction was concentrated and the residue co-evaporated with water (10 mL) followed by THF (10 mL) and DCM (10 mL). The crude product was purified by column chromatography (heptane:ethyl acetate) to afford 1.5 g (2.9 mmol, 64%) of 19 as a colourless foam.

¹H NMR (400 MHz, CDCl₃) δ 7.39-7.28 (m, 5H), 5.12 (s, 2H), 3.87 (s, 2H), 3.51 (s, 1H), 3.00 (s, 1H), 2.81 (s, 1H), 2.40 (ddd, J=15.4, 10.0, 5.1 Hz, 1H), 2.27 (ddd, J=15.8, 9.5, 6.5 Hz, 1H), 2.05-1.98 (m, 1H), 1.94-1.57 (m, 6H), 1.54-1.02 (m, 13H), 0.99 (s, 3H), 0.94 (d, J=6.4 Hz, 3H), 0.69 (s, 3H).

HRMS (ESI) m/z calcd for C₃₁H₄₅NNaO₅⁺ 534.3195, found 534.3193.

### II. Preparation of N-(benzyloxycarbonyl)-3-aza-7β-hydroxy-24-homo-5β-cholan-25-oic acid (50b)

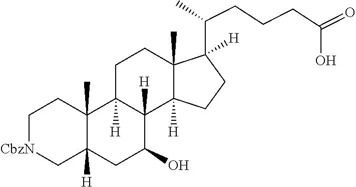

To a solution of tert-butyl N-(benzyloxycarbonyl)-3-aza-7β-hydroxy-25-homo-5β-cholan-25-oate (Compound 25b of Example 21) (930 mg, 1.6 mmol) in anhydrous DCM (25 mL, 27 vol) was added trifluoroacetic acid (TFA; 19 mL, 20 vol) at 0° C. After being stirred for 3.5 h at 0° C. the reaction was concentrated and the residue co-evaporated with water (10 mL) followed by THF (10 mL) and DCM (10 mL). The crude product was purified by column chromatography (heptane:ethyl acetate) to afford 560 mg (1.1 mmol, 69%) of 28 as a colourless foam.

¹H NMR (400 MHz, CDCl₃) δ 7.39-7.27 (m, 5H), 5.12 (s, 2H), 3.88 (s, 2H), 3.51 (s, 1H), 3.00 (s, 1H), 2.82 (s, 1H), 2.40-2.22 (m, 2H), 2.04-1.96 (m, 1H), 1.90-1.57 (m, 6H), 1.56-1.03 (m, 15H), 0.99 (s, 3H), 0.94 (d, J=6.5 Hz, 3H), 0.68 (s, 3H).

HRMS (ESI) m/z calcd for C₃₂H₄₇NNaO₅^{+ 548.3352}, found 548.3346.

### IIIa. General Procedure A for the Formation of Conjugates

Cbz-protected bile acid from step I (50a) or step II (50b) (1 equiv.) was dissolved in anhydrous DMF (25 vol). HATU (2.0 equiv.), DIPEA (3.0 equiv.) and the amine (1.2 equiv.) was added, and the reaction stirred at RT until complete by TLC. Upon completion, the reaction mixture was diluted with H₂O, extracted with ethyl acetate (×3), the organic layers were combined and washed with saturated LiCl (aq.) and concentrated. The crude residue was purified via column chromatography [C18 silica gel, gradient elution of H₂O/MeOH] to yield the conjugate as either a DIPEA salt or the free acid. DIPEA salts were treated with a sodium ion exchange column to yield the desired sodium salts of the residues.

### IIIb. General Procedure B for the Formation of Conjugates

Cbz-protected bile acid from step I (50a) or step II (50b) (1 equiv.) was dissolved in THF (25 vol) and cooled to 0° C. Ethyl chloroformate (1.2 eq) was added followed by triethylamine (1.2 eq) and the reaction stirred for 1 h at 0° C. After complete conversion of the starting material by TLC a solution of amine/thiol (1.5 eq) and NaHCO₃ (1.5 eq) in H₂O (25 vol) is added in one portion then stirred 0° C. for 2 h or until complete by TLC. Upon completion the mixture is concentrated under reduced pressure, diluted with H₂O and acidified to pH<2 with 2M HCl (aq.). The suspension was extracted (ethyl acetate or THF; ×3); the organic layers were combined and washed with brine, dried over magnesium sulphate and concentrated to afford a crude residue. The crude material was purified via column chromatography.

### IV. General Procedure C for Deprotection of Cbz Protecting Group

To a solution of Cbz-protected conjugate of Step IIIa or Step IIIb (1 equiv.) in methanol (8 mL) was added 10% palladium on charcoal (10 mol %) and the atmosphere was exchanged for hydrogen. The resulting reaction mixture was stirred at room temperature overnight. After complete conversion of the starting material by TLC the reaction was filtered and concentrated in vacuo. The crude residue was purified by column chromatography [silica gel, gradient elution of ethyl acetate/methanol:triethylamine (9:1)] to give the conjugate as a colourless foam.

### N-(3-Aza-7β-hydroxy-5β-cholan-24-amide)-ethylsulfonic acid (Compound 51a)

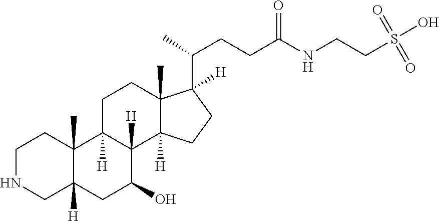

Using general procedure A compound (50a) (50 mg, 0.098 mmol) was conjugated, followed by deprotection using general procedure C, to yield compound (51a) as a colourless glass (8.5 mg, 18%).

¹H NMR (400 MHz, CD₃OD) δ 3.60 (ddt, 17.2, 13.9, 7.0 Hz, 2H), 3.45-3.33 (m, 1H), 3.30-3.01 (m, 4H), 2.96 (t, 6.8 Hz, 2H), 2.27 (ddd, 14.6, 9.6, 5.4 Hz, 1H), 2.17-2.08 (m, 1H), 2.08-1.99 (m, 2H), 1.95-1.10 (m, 18H), 1.11 (s, 3H), 0.98 (d, 6.4 Hz, 3H), 0.73 (s, 3H).

HRMS (ESI) m/z calcd for C₂₅H₄₅N₂O₅S 485.3044, found 485.3053; TLC Rf 0.27.

### N-(3-Aza-7β-hydroxy-5β-cholan-24-amide)-acetic acid (Compound 52a)

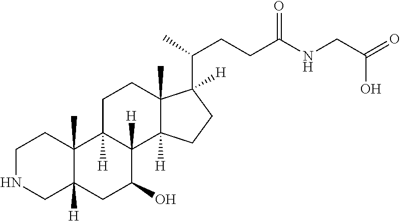

Using general procedure B compound (50a) (40 mg, 0.078 mmol) was conjugated, followed by deprotection using general procedure C, to yield compound (52a) as a colourless powder (10.4 mg, 31%).

¹H NMR (400 MHz, CD₃OD) δ 3.74 (d, 2.8 Hz, 2H), 3.44-3.34 (m, 1H), 3.20-2.93 (m, 4H), 2.30 (td, 10.6, 10.0, 5.3 Hz, 1H), 2.15 (ddd, 12.5, 9.3, 5.3 Hz, 1H), 2.11-1.96 (m, 2H), 1.88-1.21 (m, 18H), 1.09 (d, 5.9 Hz, 3H), 0.98 (d, 6.5 Hz, 3H), 0.74 (s, 3H).

HRMS (ESI) m/z calcd for C₂₅H₄₃N₂O₄⁺ 435.3217, found 435.3223.

TLC: Rf 0.16

### N-(3-Methyl-aza-7β-hydroxy-5β-cholan-24-oyl)-(2S)-2-amino-3-[(2-methylpropan-2-yl)oxy]propanoic acid (Compound 53a)

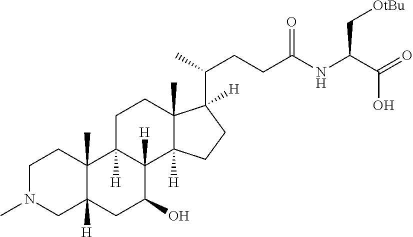

Using general procedure B compound (50a) (40 mg, 0.078 mmol) was conjugated, followed by deprotection using general procedure C, during which N-methylation occurred, to yield compound (53a) as a colourless glass (15.1 mg, 37%).

¹H NMR (400 MHz, CD₃OD) δ 4.34 (t, 3.8 Hz, 1H), 3.75 (dd, 9.1, 4.2 Hz, 1H), 3.65 (dd, 9.0, 3.5 Hz, 1H), 3.39 (dq, 8.8, 4.1, 3.4 Hz, 1H), 3.07-2.99 (m, 3H), 2.78 (t, 13.0 Hz, 1H), 2.70 (s, 3H), 2.32 (ddd, 15.0, 10.5, 5.1 Hz, 1H), 2.23-2.07 (m, 1H), 2.07-1.97 (m, 2H), 1.92-1.78 (m, 3H), 1.65-1.22 (m, 15H), 1.16 (s, 9H), 1.07 (s, 3H), 0.99 (d, 6.5 Hz, 3H), 0.73 (s, 3H).

HRMS (ESI) m/z calcd for C₃₁H₅₅N₂O₅⁺ 535.4105, found 535.4115; TLC: Rf 0.62.

### N-(3-Methyl-aza-7β-hydroxy-5β-cholan-24-oyl)-(R)-3-amino-3-phenylpropanoic acid (Compound 54a)

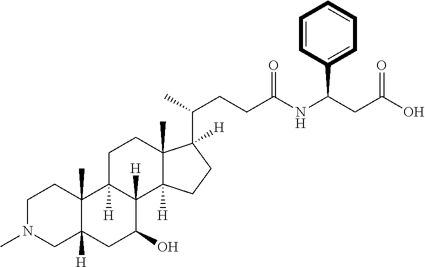

Using general procedure B compound (50a) (40 mg, 0.078 mmol) was conjugated, followed by deprotection using general procedure C, during which N-methylation occurred, to yield compound (54a) as a colourless glass (13.1 mg, 32%).

¹H NMR (400 MHz, CD₃OD) δ 7.39-7.31 (m, 2H), 7.26 (dd, 8.4, 6.8 Hz, 2H), 7.21-7.14 (m, 1H), 5.27 (t, 6.8 Hz, 1H), 3.44-3.33 (m, 1H), 3.02-2.86 (m, 3H), 2.74-2.60 (m, 5H), 2.27 (ddd, 14.2, 9.4, 5.2 Hz, 1H), 2.20-2.10 (m, 1H), 2.00 (dd, 25.4, 13.7 Hz, 2H), 1.90-1.74 (m, 5H), 1.66-1.11 (m, 14H), 1.05 (s, 3H), 0.97 (dd, 6.5, 4.2 Hz, 3H), 0.68 (s, 3H).

HRMS (ESI) m/z calcd for C₃₃H₅₁N₂O₄⁺ 539.3843, found 539.3844; TLC: Rf 0.51.

### N-(3-Methyl-aza-7β-hydroxy-5β-cholan-24-oyl)-1-amino-4-fluorobenzene (Compound 55a)

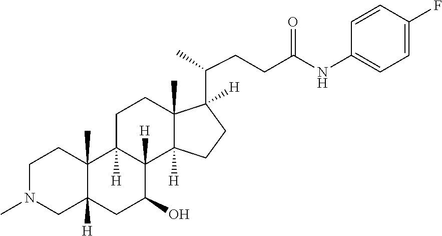

Using general procedure B compound (50a) (40 mg, 0.078 mmol) was conjugated, followed by deprotection using general procedure C, during which N-methylation occurred, to yield compound (55a) as a colourless glass (4.6 mg, 13%).

¹H NMR (400 MHz, CD₃OD) δ 7.68-7.44 (m, 2H), 7.09-6.96 (m, 2H), 3.46-3.35 (m, 1H), 2.65-2.49 (m, 2H), 2.51-2.34 (m, 2H), 2.33-2.21 (m, 4H), 2.20-2.09 (m, 1H), 2.09-1.99 (m, 1H), 1.97-1.69 (m, 6H), 1.65-1.09 (m, 13H), 1.04-0.98 (m, 6H), 0.73 (s, 3H). ¹⁹F NMR (470 MHz, CD₃OD) δ −120.83.

HRMS (ESI) m/z calcd for C₃₀H₄₆FN₂O₂⁺ 485.3538, found 485.3543; TLC Rf 0.62.

### N-(3-Aza-7β-hydroxy-5β-cholan-24-oyl)-morpholine (Compound 56a)

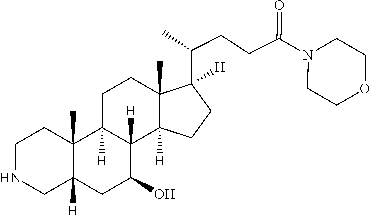

Using general procedure A compound (50a) (40 mg, 0.078 mmol) was conjugated, followed by deprotection using general procedure C, to yield compound (56a) as an off white glass (13.8 mg, 40%).

¹H NMR (400 MHz, CD₃OD) δ 3.65 (ddd, 12.5, 5.4, 3.7 Hz, 4H), 3.60-3.50 (m, 4H), 3.43-3.33 (m, 1H), 3.25-3.07 (m, 1H), 2.92 (dt, 25.2, 14.6 Hz, 3H), 2.44 (ddd, 14.6, 10.6, 5.2 Hz, 1H), 2.31 (ddd, 14.7, 10.3, 6.0 Hz, 1H), 2.06 (dt, 12.8, 2.9 Hz, 1H), 2.01-1.69 (m, 6H), 1.65-1.54 (m, 2H), 1.54-1.18 (m, 9H), 1.18-1.09 (m, 2H), 1.07 (s, 3H), 0.99 (d, 6.5 Hz, 3H), 0.74 (s, 3H).

HRMS (ESI) m/z calcd for C₂₇H₄₇N₂O₃⁺ 447.3581, found 447.3589; TLC Rf 0.20.

### N-(3-Aza-7β-hydroxy-5β-cholan-24-oyl)-(S)-2-amino-3-hydroxypropanoic acid (Compound 57a)

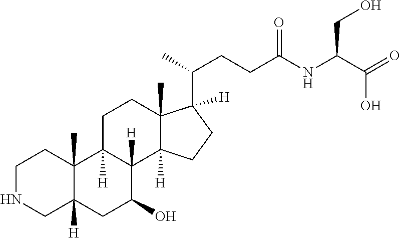

Using general procedure B compound (50a) (40 mg, 0.078 mmol) was conjugated, followed by deprotection using general procedure C, to yield compound (57a) as an off white powder (8.8 mg, 19%).

¹H NMR (400 MHz, CD₃OD) δ 4.29 (t, 4.8 Hz, 1H), 3.80 (dd, 4.8, 2.0 Hz, 2H), 3.45-3.32 (m, 1H), 3.29-2.92 (m, 4H), 2.39-2.26 (m, 1H), 2.23-2.10 (m, 1H), 2.04 (dd, 18.9, 13.2 Hz, 2H), 1.83 (d, 13.0 Hz, 3H), 1.67-1.21 (m, 14H), 1.21-1.07 (m, 4H), 0.99 (d, 6.6 Hz, 3H), 0.74 (s, 3H).

HRMS (ESI) m/z calcd for C₂₆H₄₅N₂O₅⁺ 465.3323, found 465.3322; TLC Rf 0.17.

### N-(3-Aza-7β-hydroxy-5β-cholan-24-oyl)-3-amino-2-fluoropropanoic acid (Compound 58a)

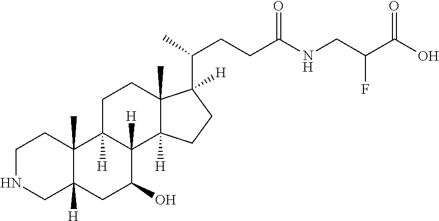

Using general procedure B compound (50a) (40 mg, 0.078 mmol) was conjugated, followed by deprotection using general procedure C, to yield compound (58a) as a colourless glass (11.2 mg, 24%).

¹H NMR (400 MHz, CD₃OD) δ 4.68 (dd, 7.6, 3.3 Hz, 1H), 3.75 (dt, 25.8, 12.4 Hz, 2H), 3.59-3.35 (m, 1H), 3.30-2.89 (m, 4H), 2.27 (ddt, 14.5, 9.8, 4.9 Hz, 1H), 2.14 (ddt, 13.6, 7.1, 3.6 Hz, 1H), 2.04 (t, 14.5 Hz, 2H), 1.86 (dd, 19.5, 9.0 Hz, 4H), 1.68-1.22 (m, 13H), 1.19-1.01 (m, 4H), 0.98 (d, 6.4 Hz, 3H), 0.73 (s, 3H). ¹⁹F NMR (470 MHz, CD₃OD) δ −187.60 (d, 17.7 Hz).

HRMS (ESI) m/z calcd for C₂₆H₄₄FN₂O₄⁺ 467.3280, found 467.3287; TLC Rf 0.22.

### N-(Cyclohexyl)-N-(3-methyl-aza-7β-hydroxy-5β-cholan-24-oyl)-cyclohexanamine (Compound 59a)

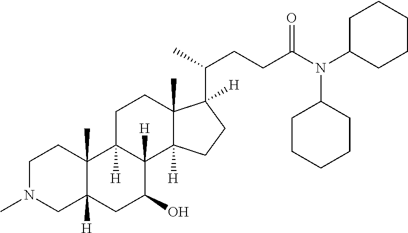

Using general procedure B compound (50a) (40 mg, 0.078 mmol) was conjugated, followed by deprotection using general procedure C, during which methylation occurred, to yield compound (59a) as an off-white glass (12.7 mg, 24%).

HRMS (ESI) m/z calcd for C₃₆H₆₃N₂O₂⁺ 555.4884, found 555.4892; TLC Rf 0.48.

### N-(3-Aza-7β-hydroxy-5β-cholan-24-oyl)-4-aminobenzoic acid (Compound 60a)

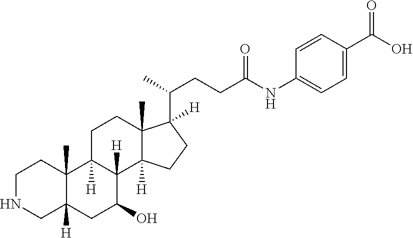

Using general procedure B compound (50a) (40 mg, 0.078 mmol) was conjugated, followed by deprotection using general procedure C, to yield compound (60a) as a yellow glass (14.2 mg, 37%).

¹H NMR (400 MHz, CD₃OD) δ 8.09-7.82 (m, 2H), 7.64-7.47 (m, 2H), 3.27-3.09 (m, 1H), 3.09-2.90 (m, 3H), 2.45 (ddd, 14.1, 9.6, 5.1 Hz, 1H), 2.30 (dt, 14.6, 7.7 Hz, 1H), 2.10-1.96 (m, 2H), 1.85 (d, 9.5 Hz, 1H), 1.61 (dd, 12.2, 4.9 Hz, 1H), 1.56-1.16 (m, 16H), 1.11 (t, 7.3 Hz, 1H), 1.07 (s, 3H), 1.03 (t, 6.4 Hz, 3H), 0.72 (s, 3H).

HRMS (ESI) m/z calcd for C₃₀H₄₅N₂O₄⁺ 497.3374, found 497.3380; TLC RF 0.12.

### N-(3-Aza-7β-hydroxy-5β-cholan-24-oyl)-(S)-2-amino-4-(methylthio)butanoic acid (Compound 61a)

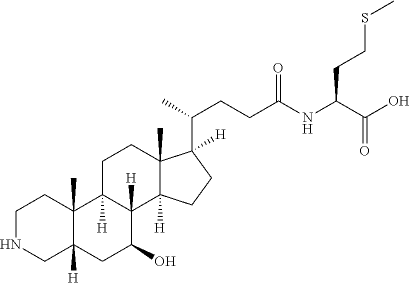

Using general procedure B compound (50a) (40 mg, 0.078 mmol) was conjugated, followed by deprotection using general procedure C, to yield compound (61a) as a colourless glass (5.2 mg, 13%); TLC Rf: 0.62.

### N-(3-Aza-7β-hydroxy-5β-cholan-24-amide)-propanoic acid (Compound 62a)

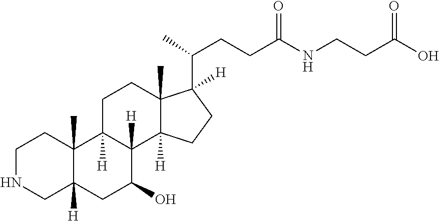

Using general procedure B compound (50a) (40 mg, 0.078 mmol) was conjugated, followed by deprotection using general procedure C, to yield compound (62a) as an off-white glass (13.8 mg, 39%).

¹H NMR (400 MHz, CD₃OD) δ 3.43-3.32 (m, 3H), 3.28-2.93 (m, 5H), 2.35 (t, 6.8 Hz, 2H), 2.23 (ddd, 13.7, 10.0, 5.3 Hz, 1H), 2.13-1.98 (m, 3H), 1.96-1.71 (m, 2H), 1.67-1.21 (m, 14H), 1.14-1.01 (m, 4H), 0.97 (d, 6.5 Hz, 3H), 0.73 (s, 3H).

HRMS (ESI) m/z calcd for C₂₆H₄₅N₂O₄⁺ 449.3374, found 449.3379; TLC Rf 0.16.

### N-(3-Aza-7β-hydroxy-5β-cholan-24-oyl)-(isopropyl-4-aminobenzoate) (Compound 63a)

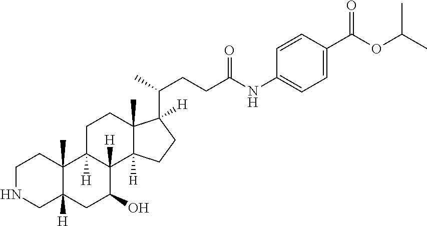

Using general procedure B compound (50a) (40 mg, 0.078 mmol) was conjugated, followed by deprotection using general procedure C, to yield compound (62a) as a colourless glass (3.2 mg, 8%).

¹H NMR (400 MHz, CD₃OD) δ 7.94 (d, 8.8 Hz, 2H), 7.67 (d, 8.8 Hz, 2H), 5.19 (p, 6.3 Hz, 1H), 3.45-3.36 (m, 2H), 3.24-2.76 (m, 6H), 2.46 (ddd, 14.7, 9.9, 5.3 Hz, 1H), 2.38-2.20 (m, 2H), 2.12-1.77 (m, 6H), 1.65-1.41 (m, 4H), 1.4-1.32 (m, 6H), 1.09 (s, 3H), 1.05 (s, 3H), 1.02 (d, 6.5 Hz, 3H), 0.96 (d, 6.5 Hz, 3H), 0.73 (s, 8.0 Hz, 3H).

HRMS (ESI) m/z calcd for C₃₃H₅₁N₂O₄⁺ 539.3843, found 539.3853; TLC Rf 0.16.

### S-(3-Aza-7β-hydroxy-5β-cholan-24-oyl)-phenylmethanethiol (Compound 64a)

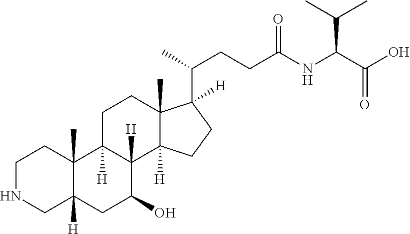

Using general procedure B compound (50a) (40 mg, 0.078 mmol) was conjugated, followed by deprotection using general procedure C, to yield compound (64a) as a colourless glass (7.0 mg, 19%).

¹H NMR (400 MHz, CD₃OD) δ 7.48-7.18 (m, 5H), 5.10 (s, 2H), 3.89-3.75 (m, 2H), 3.41 (br, 1H), 3.10 (br, 1H), 2.91 (br, 1H), 2.36 (ddd, 15.1, 9.7, 5.3 Hz, 1H), 2.30-2.16 (m, 1H), 2.08-1.99 (m, 2H), 1.95-1.72 (m, 5H), 1.63-1.05 (m, 13H), 1.01 (s, 3H), 0.95 (d, 6.5 Hz, 3H), 0.72 (s, 3H); TLC Rf 0.78.

### N-(3-Aza-7β-hydroxy-5β-cholan-24-oyl)-(S)-2-amino-3-methylbutanoic acid (Compound 65a)

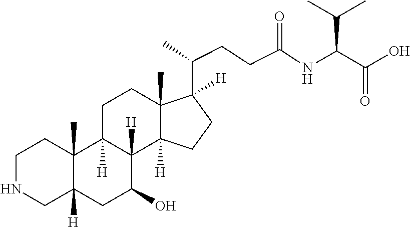

Using general procedure B compound (50a) (40 mg, 0.078 mmol) was conjugated, followed by deprotection using general procedure C, to yield compound (65a) as an off-white powder (14.9 mg, 40%).

¹H NMR (400 MHz, D₂O) δ 4.12 (d, 5.7 Hz, 1H), 3.54 (d, 8.8 Hz, 1H), 3.41-3.12 (m, 4H), 3.07 (t, 13.4 Hz, 1H), 2.52-2.38 (m, 1H), 2.33-2.19 (m, 1H), 2.19-2.04 (m, 3H), 1.93-1.74 (m, 4H), 1.68 (dd, 11.6, 5.1 Hz, 1H), 1.63-1.16 (m, 12H), 1.12 (s, 3H), 1.01 (d, 6.5 Hz, 3H), 0.96 (dd, 13.5, 6.8 Hz, 6H), 0.74 (s, 3H).

HRMS (ESI) m/z calcd for C₂₈H₄₉N₂O₄⁺ 477.3687, found 477.3691; TLC Rf 0.22.

### N-(3-Aza-7β-hydroxy-5β-cholan-24-oyl)-(2S,3S)-2-amino-3-methylpentanoic acid (Compound 66a)

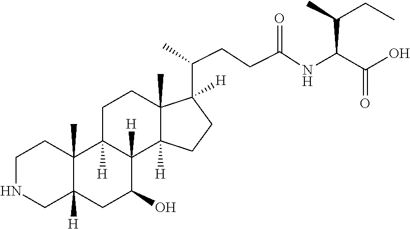

Using general procedure B compound (50a) (40 mg, 0.078 mmol) was conjugated, followed by deprotection using general procedure C, to yield compound (66a) as a colourless glass (8.0 mg, 21%).

¹H NMR (400 MHz, CD₃OD) δ 4.26 (d, 5.2 Hz, 1H), 3.42-3.35 (m, 1H), 3.25-2.93 (m, 6H), 2.38-2.25 (m, 1H), 2.23-2.21 (m, 1H), 2.11-1.97 (m, 2H), 1.91-1.78 (m, 2H), 1.71-1.22 (m, 14H), 1.21-1.06 (m, 6H), 0.99 (d, 6.5 Hz, 3H), 0.97-0.85 (m, 6H), 0.73 (s, 3H).

HRMS (ESI) m/z calcd for C₂₉H₅₁N₂O₄⁺ 491.3843, found 491.3853; TLC Rf 0.24.

### N-Methyl-N-(3-aza-7β-hydroxy-5β-cholan-24-oyl)-glycine (Compound 67a)

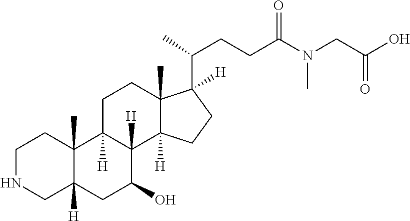

Using general procedure B compound (50a) (40 mg, 0.078 mmol) was conjugated, followed by deprotection using general procedure C, to yield compound (67a) as a colourless glass (9.9 mg, 28%).

¹H NMR (400 MHz, CD₃OD) δ 3.87 (s, 2H), 3.28-3.01 (m, 5H), 2.95 (s, 3H), 2.40-2.26 (m, 1H), 2.26-2.12 (m, 1H), 2.05 (dd, 18.1, 9.3 Hz, 4H), 1.89-1.69 (m, 1H), 1.64-1.23 (m, 15H), 1.19-1.05 (m, 4H), 0.97 (d, 6.5 Hz, 3H), 0.73 (s, 3H).

HRMS (ESI) m/z calcd for C₂₆H₄₅N₂O₄⁺ 449.3374, found 449.3381; TLC Rf 0.16. N-(3-Aza-7β-hydroxy-5β-cholan-24-oyl)-(S)-2-aminopropanoic acid (Compound 68a)

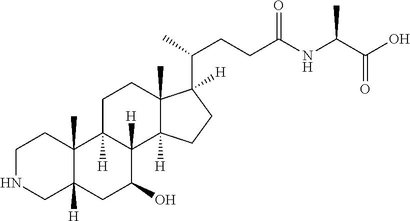

Using general procedure B compound (50a) (40 mg, 0.078 mmol) was conjugated, followed by deprotection using general procedure C, to yield compound (68a) as a colourless glass (10.3 mg, 29%).

¹H NMR (400 MHz, CD₃OD) δ 4.20 (q, 7.1 Hz, 1H), 3.45-3.33 (m, 1H), 3.27-3.17 (m, 2H), 3.17-2.84 (m, 3H), 2.28 (ddd, 13.9, 10.4, 5.3 Hz, 1H), 2.19-2.10 (m, 1H), 2.10-1.96 (m, 2H), 1.96-1.75 (m, 4H), 1.66-1.24 (m, 16H), 1.09 (s, 3H), 0.98 (d, 6.5 Hz, 3H), 0.73 (s, 3H).

HRMS (ESI) m/z calcd for C₂₆H₄₅N₂O₄⁺ 449.3374, found 449.3380; TLC Rf 0.18.

### N-(3-Aza-7β-hydroxy-25-homo-5β-cholan-25-oyl)-(S)-2-aminobutanedioic acid (Compound 70b)

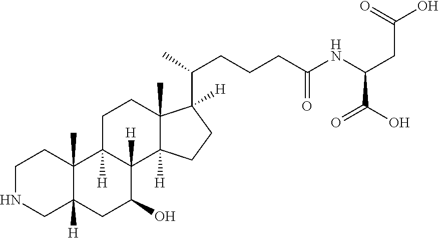

Using general procedure B compound (50b) (40 mg, 0.076 mmol) was conjugated, followed by deprotection using general procedure C, to yield compound (70b) as an off-white powder (7.5 mg, 19%).

HRMS (ESI) m/z calcd for C₂₈H₄₇N₂O₆⁺ 507.3429, found 507.3436; TLC Rf 0.04.

### N-(3-Aza-7β-hydroxy-25-homo-5β-cholan-25-oyl)-(2S)-2-amino-3-[(2-methylpropan-2-yl)oxy]propanoic acid (Compound 71b)

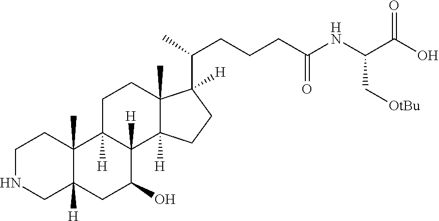

Using general procedure B compound (50b) (40 mg, 0.076 mmol) was conjugated, followed by deprotection using general procedure C, to yield compound (71b) as an off white powder (4.6 mg, 11%).

¹H NMR (400 MHz, CD₃OD) δ 4.35 (t, 3.9 Hz, 2H), 3.75 (dd, 9.0, 4.3 Hz, 1H), 3.65 (dd, 8.8, 3.5 Hz, 1H), 3.39-2.92 (m, 5H), 3.09-2.95 (m, 1H), 2.29-2.16 (m, 2H), 2.10-1.96 (m, 2H), 1.61-1.20 (m, 18H), 1.16 (d, 1.4 Hz, 9H), 1.08 (s, 3H), 0.97 (d, 6.1 Hz, 3H), 0.73 (s, 3H).

HRMS (ESI) m/z calcd for C₃₁H₅₅N₂O₅⁺ 535.4105, found 535.4096; TLC Rf 0.24.

### N-(3-Aza-7β-hydroxy-25-homo-5β-cholan-25-amide)-ethylsulfonic acid (Compound 72b)

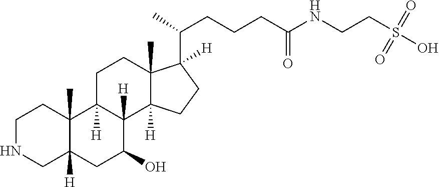

Using general procedure B compound (50b) (40 mg, 0.076 mmol) was conjugated, followed by deprotection using general procedure C, to yield compound (72b) as a colourless glass (5.2 mg, 14%).

¹H NMR (400 MHz, CD₃OD) δ 3.59 (t, 6.8 Hz, 2H), 3.28-3.00 (m, 5H), 2.96 (t, 6.8 Hz, 2H), 2.22-1.97 (m, 4H), 1.96-1.77 (m, 2H), 1.61-1.10 (m, 18H), 1.08 (s, 3H), 0.96 (d, 6.6 Hz, 3H), 0.73 (s, 3H).

HRMS (ESI) m/z calcd for C₂₆H₄₇N₂O₅S 499.3200, found 499.3207; TLC Rf 0.25.

### N-(3-Aza-7β-hydroxy-25-homo-5β-cholan-25-oyl)-(S)-2-amino-3-phenylpropanoic acid (Compound 73b)

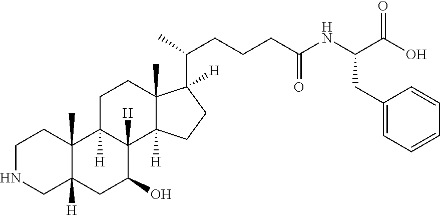

Using general procedure B compound (50b) (40 mg, 0.076 mmol) was conjugated, followed by deprotection using general procedure C, to yield compound (73b) as an off-white powder (18.7 mg, 46%).

¹H NMR (400 MHz, CD₃OD) δ 7.36-7.04 (m, 5H), 4.52 (dd, 7.6, 4.9 Hz, 1H), 3.39 (dd, 10.6, 5.1 Hz, 1H), 3.27-3.14 (m, 4H), 3.11-2.93 (m, 3H), 2.19-1.95 (m, 4H), 1.89-1.74 (m, 4H), 1.68-1.13 (m, 15H), 1.08 (s, 3H), 0.92 (d, 6.5 Hz, 3H), 0.72 (s, 3H).

HRMS (ESI) m/z calcd for C₃₃H₅₁N₂O₄⁺ 539.3843, found 539.3849; TLC Rf 0.21.

### N-(3-Aza-7β-hydroxy-25-homo-5β-cholan-25-oyl)-3-aminobutanoic acid (Compound 74b)

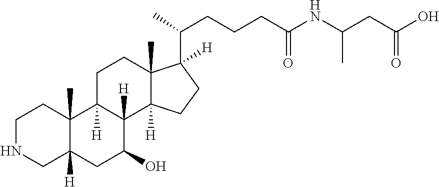

Using general procedure B compound (50b) (40 mg, 0.076 mmol) was conjugated, followed by deprotection using general procedure C, to yield compound (74b) as an off-white glass (12.2 mg, 34%).

¹H NMR (400 MHz, CD₃OD) δ 4.21 (h, 6.6 Hz, 1H), 3.44-3.34 (m, 1H), 3.29-2.89 (m, 6H), 2.38 (dd, 14.3, 5.7 Hz, 1H), 2.26 (dd, 14.3, 7.2 Hz, 1H), 2.19-1.96 (m, 3H), 1.90-1.77 (m, 1H), 1.77-1.22 (m, 18H), 1.18 (d, 6.5 Hz, 3H), 1.09 (s, 3H), 0.96 (d, 6.5 Hz, 3H), 0.73 (s, 3H).

HRMS (ESI) m/z calcd for C₂₈H₄₉N₂O₄⁺ 477.3687, found 477.3678; TLC Rf 0.15.

### N-(3-Aza-7β-hydroxy-25-homo-5β-cholan-25-oyl)-(S)-2-amino-4-methylpentanoic acid (Compound 75b)

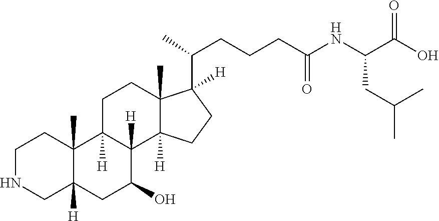

Using general procedure B compound (50b) (40 mg, 0.076 mmol) was conjugated, followed by deprotection using general procedure C, to yield compound (75b) as a white powder (4.5 mg, 12%).

¹H NMR (400 MHz, CD₃OD) δ 4.36 (dd, 9.7, 4.0 Hz, 1H), 3.27-2.91 (m, 5H), 2.28-1.94 (m, 3H), 1.89-1.76 (m, 2H), 1.74-1.20 (m, 22H), 1.08 (s, 3H), 0.99-0.90 (m, 9H), 0.73 (s, 3H).

HRMS (ESI) m/z calcd for C₃₀H₅₃N₂O₄⁺ 505.4000, found 505.4007; TLC Rf 0.20.

### N-(3-Aza-7β-hydroxy-25-homo-5β-cholan-25-oyl)-(2S)-2,6-diaminohexanoic acid (Compound 76b)

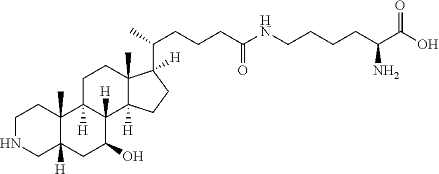

Using general procedure B compound (50b) (40 mg, 0.076 mmol) was conjugated, followed by deprotection using general procedure C, to yield compound (76b) as a yellow glass (12.9 mg, 33%).

¹H NMR (400 MHz, CD₃OD) δ 3.54-3.45 (m, 1H), 3.44-3.35 (m, 1H), 3.28-2.74 (m, 6H), 2.25-1.96 (m, 5H), 1.92-1.70 (m, 3H), 1.70-1.18 (m, 24H), 1.09 (s, 3H), 0.97 (d, 6.4 Hz, 3H), 0.73 (s, 3H).

HRMS (ESI) m/z calcd for C₃₀H₅₄N₃O₄⁺ 520.4109, found 520.4114; TLC Rf 0.07.

### N-(3-Aza-7β-hydroxy-25-homo-5β-cholan-25-oyl)-(2S)-2-amino-3-(4-hydroxyphenyl)propanoic acid (Compound 77b)

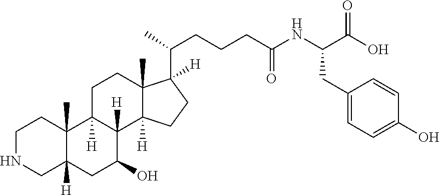

Using general procedure B compound (50b) (40 mg, 0.076 mmol) was conjugated, followed by deprotection using general procedure C, to yield compound (77b) as a yellow glass (11.0 mg, 26%).

¹H NMR (400 MHz, CD₃OD) δ 7.03 (d, 8.5 Hz, 2H), 6.66 (d, 8.5 Hz, 2H), 4.47 (dd, 7.3, 4.9 Hz, 1H), 3.28-2.80 (m, 7H), 2.19-1.97 (m, 2H), 1.90-1.78 (m, 3H), 1.67-1.19 (m, 19H), 1.09 (s, 3H), 0.93 (d, 6.5 Hz, 3H), 0.72 (s, 3H).

HRMS (ESI) m/z calcd for C₃₃H₅₁N₂O₅⁺ 555.3792, found 555.3803; TLC Rf 0.14.

### N-{(benzyloxycarbonyl)-3-aza-7β-hydroxy-5β-cholan-24-oyl}-2-oxa-6-azospiro{3,3}heptane (Compound 78a)

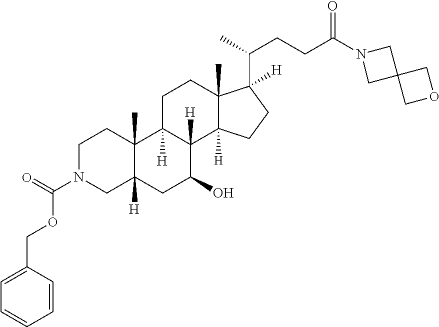

Using general procedure B compound (50a) (33 mg, 0.06 mmol) was conjugated, to yield compound (78a) as a colourless glass (17 mg, 45%).

¹H NMR (400 MHz, CDCl₃) 7.38-7.28 (m, 5H), 5.11 (br. s, 2H), 4.80 (d, J=5.8 Hz, 2H), 4.77 (d, J=5.4 Hz, 2H), 4.26 (s, 2H), 4.13 (s, 2H), 3.97-3.77 (br. m, 2H), 3.57-3.45 (br. m, 1H), 3.07-2.91 (br. m, 1H), 2.89-2.73 (br. m, 1H), 2.15-2.05 (m, 1H), 2.05-1.98 (m, 1H), 1.98-1.86 (m, 2H), 1.83-1.73 (m, 4H), 1.67-1.62 (m, 2H), 1.52-1.38 (m, 5H), 1.35-1.27 (m, 3H), 1.24-1.03 (m, 4H), 0.99 (s, 3H), 0.92 (d, J=6.5, 3H), 0.69 (s, 3H). ¹³C NMR (100 MHz, CDCl₃) 173.6, 155.4, 137.0, 128.5 (2×C), 127.9, 127.8 (2×C), 80.8 (2×C), 71.1, 67.0, 59.8, 57.2, 56.4, 55.7, 55.0, 44.4, 43.8, 43.5, 42.7, 40.0, 39.3, 38.7, 37.6, 35.5, 34.3, 33.6, 30.7, 28.6, 28.4, 26.8, 23.4, 21.3, 18.5, 12.2.

### N-{(benzyloxycarbonyl)-3-aza-7β-hydroxy-5β-cholan-24-oyl}-4-piperidone (Compound 79a)

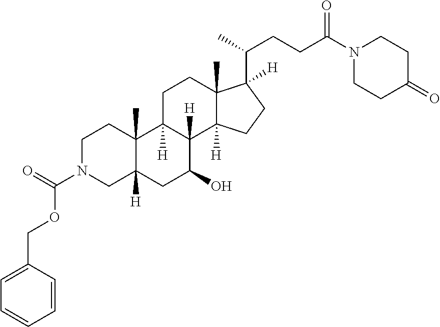

Using general procedure B compound (50a) (40 mg, 0.08 mmol) was conjugated, to yield compound (79a) as a colourless glass (20 mg, 43%).

¹H NMR (400 MHz, CDCl₃) 7.38-7.28 (m, 5H), 5.11 (br. s, 2H), 3.96-3.79 (br. m, 2H), 3.88 (br. t, J=6.0 Hz, 2H), 3.75 (br. t, J=6.0 Hz, 2H), 3.57-3.46 (br. m, 1H), 3.08-2.92 (br. m, 1H), 2.89-2.73 (br. m, 1H), 2.49-2.42 (m, 5H), 2.31 (ddd, J=15.0, 10.5, 5.9 Hz, 1H), 2.05-1.99 (m, 1H), 1.97-1.89 (m, 1H), 1.89-1.71 (m, 3H), 1.69-1.55 (m, 2H⁺H₂O), 1.53-1.30 (m, 8H), 1.28-1.12 (m, 4H), 1.18-1.14 (m 1H), 0.99 (s, 3H), 0.96 (d, J=6.4, 3H), 0.70 (s, 3H).

¹³C NMR (100 MHz, CDCl₃) 206.8, 172.3, 155.4, 136.9, 128.5 (2×C), 127.9, 127.8 (2×C), 71.1, 67.0, 55.7, 55.1, 44.4, 44.2, 43.8, 43.5, 42.7, 41.3 (2×C), 40.9, 40.0, 39.3, 38.7, 35.5, 34.3, 33.6, 31.4, 30.3, 29.7, 28.7, 26.9, 23.4, 21.3, 18.6, 12.2.

### N-{(benzyloxycarbonyl)-3-aza-7β-hydroxy-25-homo-5β-cholan-25-oyl}-3-aminotetrahydrofuran (Compound 80b)

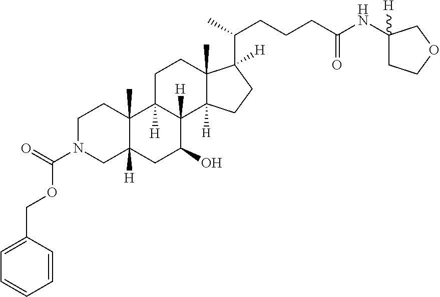

Using general procedure B compound (50b) (35 mg, 0.07 mmol) was conjugated, to yield compound (80b) as a colourless glass (18 mg, 44%).

¹H NMR (400 MHz, CDCl₃) 7.38-7.28 (m, 5H), 5.69 (d, J=7.4 Hz, 1H, NH), 5.12 (br. s, 2H), 4.53 (m, 1H), 3.96-3.75 (br. m, 5H), 3.65 (dd, J=9.4, 2.5, 1H), 3.57-3.46 (br. m, 1H), 3.08-2.92 (br. m, 1H), 2.89-2.73 (br. m, 1H), 2.32-2.21 (m, 1H), 2.19-1.98 (m, 3H), 1.92-1.58 (m, 8H), 1.56-1.33 (m, 5H⁺H₂O), 1.33-1.17 (m, 6H), 1.16-1.02 (m, 3H), 0.99 (s, 3H), 0.94 (d, J=6.5, 3H), 0.68 (s, 3H).

### N-{(3-aza-7β-hydroxy-5β-cholan-25-oyl}-isoindoline (Compound 81a)

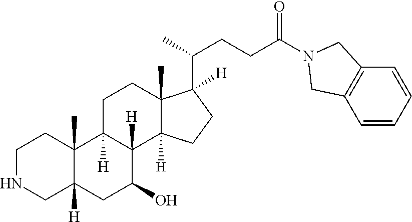

Using general procedure B compound (50a) (40 mg, 0.08 mmol) was conjugated followed by deprotection using general procedure C, to yield compound (81a) as a pale yellow glass (5.6 mg, 15%).

¹H NMR (400 MHz, CD₃OD) 7.37-7.27 (m, 4H), 4.90 (br. s, 2H), 4.74 (br. s, 2H), 3.43-3.34 (m, 1H), 3.28-2.95 (m, 4H), 2.51 (ddd, J=15.6, 10.6, 5.4 Hz, 1H), 2.37 (ddd, J=15.6, 10.1, 6.0 Hz, 1H), 2.13-1.99 (m, 2H), 1.97-1.81 (m, 5H), 1.66-1.22 (m, 1.10, 12H), 1.22-1.14 (m, 1H), 1.10 (s, 3H), 1.04 (d, J=6.6 Hz, 3H), 0.76 (s, 3H).

¹³C NMR (100 MHz, CD₃OD) 175.3, 137.7, 137.3, 128.9, 128.7, 124.0, 123.8, 71.1, 57.0, 56.7, 53.7, 53.2, 45.0, 44.8, 44.3, 42.2, 41.2, 40.8, 40.0, 37.0, 35.2, 34.3, 33.7, 32.4, 32.2, 29.7, 27.9, 23.5, 22.5, 19.2, 12.6.

### N-{(3-aza-7β-hydroxy-25-homo-5β-cholan-25-oyl)}-3-aminotetrahydrofuran (Compound 82b)

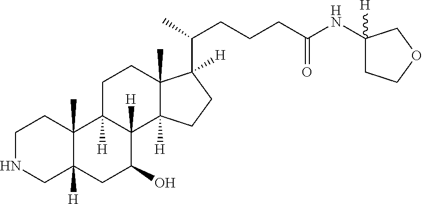

Using general procedure C compound (80b) (17.6 mg, 0.03 mmol) was deprotected to yield compound (82b) as a pale yellow glass (4.0 mg, 29%).

¹H NMR (400 MHz, CD₃OD) 4.38-4.32 (m, 1H), 3.94-3.75 (m, 3H), 3.58 (dd, J=9.1, 3.6 Hz, 1H), 3.42-3.34 (m, 1H), 3.12-2.99 (m, 1H), 2.95-2.76 (m, 3H), 2.25-2.09 (m, 3H), 2.09-2.00 (m, 2H), 1.95-1.77 (m, 4H), 1.76-1.65 (m, 2H), 1.64-1.54 (m, 2H), 1.54-1.38 (m, 7H), 1.38-1.17 (m, 5H), 1.16-1.04 (m, 1H), 1.05 (s, 3H), 0.96 (d J=6.6 Hz, 3H), 0.72 (s, 3H).

¹³C NMR (100 MHz, CD₃OD) 176.3, 74.1, 71.5, 68.0, 57.2, 56.6, 51.5, 46.3, 44.8, 44.4, 43.7, 41.4, 41.3, 40.2, 37.2, 36.8, 35.8, 34.6, 33.3, 30.8, 29.7, 27.9, 24.0, 23.8, 23.6, 22.5, 19.3, 12.6.

### Biological Example

### A. Culture of Primary Fibroblasts, Generation and Culture of iNPC's

Primary fibroblast cells were cultured continuously in Glucose based medium (high glucose (4500 mg/L) Dulbecco's Modified Eagle's medium (DMEM; Sigma-Aldrich) supplemented with 10% fetal bovine serum (Sigma-Aldrich), 100 IU/mL penicillin, 100 μg/mL streptomycin (Lonza), 1 mM sodium pyruvate (Sigma-Aldrich) and 50 μg/mL uridine (Sigma-Aldrich)). Unless otherwise stated, 24 hours prior to analysis, the Glucose based medium was exchanged for Galactose based medium (glucose-free DMEM (Gibco) with the same supplementation and in addition, 5 mM galactose (Sigma-Aldrich)). All cells were assessed between passage 6-10.

Induced neural progenitor cells (iNPC's) were generated as previously described (Meyer et al, “Direct conversion of patient fibroblasts demonstrates non-cell autonomous toxicity of astrocytes to motor neurons in familial and sporadic ALS” Proc Natl Acad Sci USA 2014). iNPC's were maintained in DMEM/Ham F12 (Invitrogen); N₂, B27 supplements (Invitrogen) and FGFb (Peprotech) in fibronectin (Millipore) coated tissue culture dishes and routinely subcultured every 2-3 days using accutase to detach them. The fibroblasts lines and iNPC lines have previously been characterised and published in Carling et al, 2020.

### B. Dopaminergic Neuron Differentiation of iNPC's

Briefly, iNPCs are plated in a 6-well plate and cultured for 2 days in DMEM/F-12 medium with Glutamax™ supplemented with 1% NEAA, 2% B27 (Gibco) and 2.5 μM of DAPT. On day 3, DAPT is removed and the medium is supplemented with 1 μM smoothened agonist (SAG) and FGF8 (75 ng/ml) for additional 10 days. Neurons are replated at this stage. Subsequently SAG and FGF8 are withdrawn and replaced with BDNF (30 ng/ml), GDNF (30 ng/ml), TGF-b3 (2 mM) and dcAMP (2 mM, Sigma) for 15 days, as previously described Schwartzentruber et al, 2020 and Carling et al, 2020. Dopaminergic neurons were treated with compounds at concentrations of 0.1 nM, 1 nM, 3 nM, 10 nM, 30 nM and 100 nM dosing every 3 days for the last 12 days of differentiation.

### C. Immunofluorescence Staining

Cells are plated into 96 well plates and fixed using 4% paraformaldehyde for 30 minutes. After PBS washes cells are permeabilised using 0.1% Triton™ X-100 for 10 minutes and blocked using 5% goat serum for 1 hour. Cells are incubated with primary antibodies βIII tubulin (Millipore); activated caspase 3 (Cell Signaling); at 4° C. for 16 hours. Cells are washed using PBS-Tween® and incubated with Alexa Fluor™-conjugated secondary antibodies 488 and 568 (Invitrogen) and Hoescht (Sigma) 1 μM prior to imaging. Imaging was performed using the Opera Phenix™ high content imaging system (Perkin Elmer).

### MMP Protocol

Fibroblasts were cultured and plated into a greiner black 384 μClear® plate at a concentration of 1000 cells per well in 50 μl of media volume. The plates are left overnight in an incubator to allow the fibroblasts to adhere to the plate surface. The following morning the Glucose based medium is replaced with 25 μl of Galactose based medium. The plates were then dosed with the compounds using an ECHO® 550 liquid handling system. The wells were dosed to provide an 8-point concentration range of 0.06 nM-300 nM of compound. After dosing the wells are topped up with a further 25 μl of Galactose based medium and then left in an incubator for 24 hours. After 24 hours, the medium is removed from the wells and replaced with 25 μl phenol free Minimal essential medium supplemented with 10% FBS, 1% Penicillin-streptomycin, 1% sodium pyruvate, 0.1% uridine, 1% non-essential amino acids and 1% MEM vitamins with 80 nM TMRM (Sigma) and 10 μM Hoechst Stain (Sigma). The plate is returned to the incubator for another hour after which the stain medium is removed and replaced with 25 μl Phenol free MEM. The plate is then imaged using an IN Cell high content microscope (GE Healthcare) with fields of view per well in 2 channels, Cy3 excitation 542 nm, emission 604-64 nm; and the DAPI excitation 350 nm, emission 450-55 nm at 37° C. with CO₂. After imaging the plate is disposed of and the images are Data mined using the INCell developer Toolkit (GE Healthcare).

### Full ATP Protocol

The ATP protocol is generally as described in Mortiboys et al 2008. Briefly, fibroblasts were cultured as and plated into white 384 well plates at a concentration of 5000 cells per well in 50 μl of media volume. The plates are left overnight in an incubator to allow the fibroblasts to adhere to the plate surface. The following morning the Glucose based medium is replaced with 25 μl of Galactose based medium. The plates were then spiked with the compounds using a ECHO 550 liquid handling system. The wells were dosed to provide an 8-point concentration range of 0.06 nM-300 nM of compound. After dosing the wells are topped up with a further 25 μl of Galactose based medium and then left in an incubator for 24 hours. Following this incubation, the medium is removed from the plate and the wells are washed twice with sterile PBS. The wells are filled with 25 μl of Sterile PBS followed by 12.5 μl of Lysis solution from the ATPlite™ Luminescence ATP detection assay system (Perkin Elmer), including 16 cell free wells to use as blank controls. The plate is then placed on a rotary shaker for 5 mins at 700 rpm. Following the shaking 12.5 μl of ATP substrate solution (Perkin Elmer) is added to each well and a further 5 min of shaking. The plate is then placed in darkness for 10 minutes prior to reading. Using a PHERAStar® plate reader, luminescence intensity is recorded. Following the ATP assay the plates are immediately assayed for DNA content in a CyQUANT® assay.

Immediately following The ATP assay DNA content is assessed with the CyQUANT® NF Cell Proliferation Assay Kit (ThermoFisher). CyQUANT® buffer is prepared immediately before the assay and is comprised of 1 μl CyQUANT® dye per ml×1 HBSS solution. 12.5 μl of CyQUANT® buffer is added to each well. Plate left in incubator for 1 hour then read on a PHERAStar© Plate reader with excitation at 497 nm and emission at 520 nm.

ATP Quantification for each well is determined using the following formula:

$$ATP score = \frac{ATPlite blank corrected value}{CyQUANT blank corrected value}$$

### Data Analysis for Primary Screen Assays.

After the assays had been repeated in triplicate per line and compound the data was then inputted into Graph pad Prism 7 software suite where a dose response curve is generated using the default “[Agonist] vs response(three parameters)” equation.

$$y = Bottom + x \times \frac{Top - Bottom}{EC 50 + x}$$

From this EC₅₀ values, lowest response and maximal response were taken and used to calculate the Geometric mean between the 5 different lines assessed.

Results derived from compounds that showed an Ambiguous result from the “[Agonist] vs response (three parameters)” equation were excluded from the Geometric mean calculations due to the high skew that was introduced by their inclusion.

### Preliminary ATP Protocol

In some cases, a preliminary ATP protocol was used in which EC₅₀ was not determined. The preliminary ATP protocol is generally as described in Mortiboys et al 2008. Briefly, fibroblasts were cultured and plated into white, clear-bottom 384 well plates at a concentration of 4000 cells per well in 50 μl of media volume. The plates are left overnight in an incubator to allow the fibroblasts to adhere to the plate surface. The following morning the Glucose based medium is replaced with 25 μl of Galactose based medium. The cells were then dosed with 100 nM and 1 μM concentrations of the compounds using a ECHO 550 liquid handling system. After dosing the wells are topped up with a further 25 μl of Galactose based medium and then left in an incubator for 24 hours. Following this incubation, the medium is removed from the plate and the wells are washed twice with sterile PBS. The wells are filled with 20 μl of Sterile PBS followed by 10 μl of Lysis solution from the ATPlite™ Luminescence ATP detection assay system (Perkin Elmer), including 14 cell free wells to use as blank controls. The plate is then placed on a rotary shaker for 10 mins at 700 rpm. Following the shaking 10 μl of ATP substrate solution (Perkin Elmer) is added to each well and a further 5 min of shaking. The plate is then incubated in the dark for 10 minutes prior to reading. Using a FLUOstar® Omega plate reader, luminescence intensity is recorded. Following the ATP assay the plates are immediately assayed for DNA content in a CyQUANT® assay.

Immediately following The ATP assay DNA content is assessed with the CyQUANT® NF Cell Proliferation Assay Kit (ThermoFisher). CyQUANT® buffer is prepared immediately before the assay and is comprised of 2 μl CyQUANT® dye per ml×1 HBSS solution. 10 μl of CyQUANT® buffer is added to each well. Cell plates were then incubated for 1 hour before being read on a FLUOStar® Plate reader with excitation at 497 nm and emission at 520 nm.

The ATP Quantification for each well is determined as set out above.

### Respiration Measurements

Oxygen consumption rate (OCR) was measured by the Agilent Seahorse Mito Stress test using a 24-well Agilent Seahorse XF analyzer machine (Agilent). Human fibroblasts where plated at a density of 60,000 cells per well. Cells were treated with compound 50 nM for 24 hours prior to measurement. 3

measurements of OCR were taken in each state: basal state, after addition of oligomycin (0.5 μM), FCCP (0.5 μM) and rotenone (1 μM). A cell count was then done on a fixed assay plate using a Hoechst dye (1 μM). Data presented is normalized to cell number.

### Complex I Assay

Ex vivo mice brain was homogenated in a buffer of 250 mM sucrose, 20 mM HEPES, 3 mM EDTA, pH 7.5 at 4° C. Homogenisation was carried out using a Dounce homogenizer, for cortex samples, and by repetitive passage through a 0.5 mm syringe for isolated striatum. Samples were then incubated with 30 μl of detergent from the AbCam colorimetric Complex I assay kit on ice for 20 minutes. Samples are then centrifuged at 13,000 rpm for 30 mins. Triplicate samples per condition were blocked using the kit blocking buffer on the AbCam colorimetric Complex I assay kit plate for 3 hours. Samples are then washed using the kit wash buffer 3 times before the addition of the kit assay buffer containing NADH and colorimetric dye. The assay plate is read on a plate reader in a kinetic assay programme reading 450 nm in a 30 second interval for 50 minutes.

D. Mitochondrial Function and Morphology Measurements in iNeurons

iNeurons were treated every 3 days for the last 12 days of the differentiation protocol. Cells are plated in 96 well plates; for live imaging cells are incubated for one hour at 37° C. with 80 nM tetramethlyrhodamine (TMRM), 1 μM LysoTracker® Green (Invitrogen) and Hoechst Stain solution (Sigma) at 1 μM before imaging using Opera Phenix™. Cellular ATP measurements are undertaken using ATPlite kit (Perkin Elmer) as per manufacturer's instructions. Mitochondrial reactive oxygen species generation was assessed using mitochondrial NpFR2 (probe; a kind gift from Dr Liz New, University of Sydney, Australia) at 20 μM and Hoechst stain solution at 1 μM for 30 mins at 37° C., then the dyes are removed and cells images using Opera Phenix™. Images generated from the live imaging experiments were analysed using Harmony® (Perkin Elmer software). We developed protocols in order to segment nucleus, cell boundary and processes, mitochondria, lysosomes, autophagosomes. We only analysed the z projection images collected from the z stacks.

### Results

### Fibroblasts

The mitochondrial membrane potential was measured in fibroblasts from 3 patients with sporadic Parkinson's disease (sPD) when treated with Compounds of the invention. The results are shown in Table 1, where “Bottom”=max response with lowest dose of compound (0.06 nM) and “top”=max response with highest dose of compound (300 nM).

TABLE 1
Mitochondrial Membrane Potential data
from 3 sporadic PD patient fibroblasts

| | MMP % of vehicle | | |
|---|---|---|---|
| Example | bottom % | top % | EC₅₀ (nM) |
| 28a | 96.8 | 119.8 | 4.58 |
| 28b | 92.8 | 100.6 | 4.03 |
| 40a | 93.5 | 105.6 | 65.3 |
| 41b | 84.6 | 91.13 | 17.6 |
| 40b | 95.3 | 130.83 | 26.08 |
| 41b | 94.3 | 123.23 | 2.8 |
| 42a | 71.3 | 69.2 | 129.6 |
| 42b | 92.2 | 96.6 | 5.1 |
| 43a | 84.1 | 100.1 | 0.2 |
| 43b | 86.9 | 102.6 | 1.1 |
| 44a | 73.5 | 101.5 | 0.09 |
| 44b | 77.8 | 98.8 | 0.11 |
| 45a | 82.3 | 95.1 | 0.18 |
| 33 | 82.3 | 92.2 | 18.3 |
| 31b | 86.5 | 95.1 | 6.1 |
| 32 | 92.6 | 121.8 | 5.4 |

NB: The sPD patients have a mean reduction in MMP compared to controls of 18%; therefore increase of MMP from the vehicle treated sPD patient level of 118% would restore MMP to control levels.

Cellular ATP levels were measured in fibroblasts from 3 patients with sporadic Parkinson's disease when treated with Compounds of the invention. The results are shown in Table 2, where “Bottom”=max response with lowest dose of compound (0.06 nM) and “top”=max response with highest dose of compound (300 nM).

TABLE 2
Cellular ATP levels data from 3 sporadic
PD patient fibroblasts in full ATP assay

| | ATP % of vehicle | | |
|---|---|---|---|
| Example | bottom % | top % | EC₅₀ (nM) |
| 28a | 98.3 | 137.03 | 10.9 |
| 28b | 94.6 | 138.12 | 32.9 |
| 40a | 89.9 | 103.1 | 22.8 |
| 41b | 78.9 | 99.3 | 33.7 |
| 40b | 101.3 | 129.7 | 19.53 |
| 41b | 76.4 | 120.8 | 3.13 |
| 42a | 89.6 | 64.5 | 133.7 |
| 42b | 91.3 | 98.5 | 25.4 |
| 43a | 90.2 | 85.98 | 8.9 |
| 43b | ~ | 80.6 | ~ |
| 44a | 65.1 | 122.4 | 0.23 |
| 44b | ~ | 98.4 | ~ |
| 45a | 83.8 | 97.2 | 0.25 |
| 33 | 88.8 | 103.1 | 0.75 |
| 31b | 91.7 | 160.8 | 0.266 |
| 32 | 93.2 | 140.2 | 1.6 |

NB: sPD fibroblasts have an average reduction of 24% of cellular ATP levels as compared to controls. Therefore a % of vehicle treated sPD fibroblasts of 124% is an increase to control ATP levels.

The MMP and ATP assays described above along with a toxicity measure comprise the primary screen of the Compounds in primary patient fibroblasts. When considering which compound is most active in the primary screens all information is taken into account including EC50 values indicating potency and % maximal responses for both assays; based upon the combined activity expert biologists take decisions for each compound.

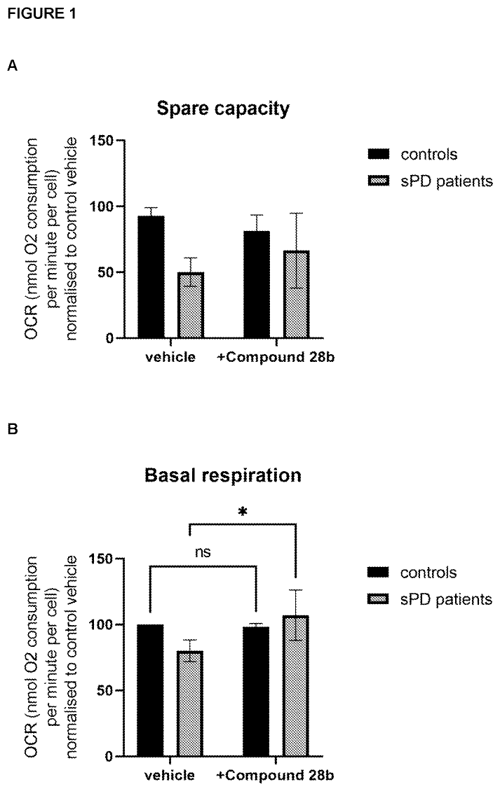

Oxygen Consumption data from 3 sporadic PD patient fibroblast lines and 3 controls are shown in FIGS. 1A, 1B and 1C, from which it can be seen that when treated with vehicle, sPD fibroblasts show a reduction of basal mitochondrial respiration of 30%, spare respiratory capacity of 42% and ATP linked respiration of 23% compared with the control cell lines. Treatment with Compound 28b increases basal respiration in the sPD fibroblasts to control levels (* p<0.05). Increases are also seen in ATP linked respiration and spare capacity in the sPD fibroblasts although to a lesser extent. Note Compound 28b was dosed at 50 nM and therefore it is clear Compound 28b provides an increase in mitochondrial function as measured by oxygen consumption.

The above data shows the mitochondrial protective effects of the compounds in primary fibroblasts from sPD patients however the cell type which is primarily affected in PD is the dopaminergic neuron. The data below shows the results obtained from dopaminergic neurons derived from sPD patients; these cultures are approximately 96% dopaminergic neurons and currently this methodology is the only protocol to generate such a pure dopaminergic culture from patient cells (method developed by Mortiboys, University of Sheffield published previously Schwarztentruber et al, 2020, Carling et al, 2020); therefore this is the patient derived model which represents most closely the neurons affected in PD.

Table 3 below shows the results for mitochondrial function and neuronal morphology measurements in iNeurons from sPD patients vs controls when untreated or when treated with either UDCA, Compound 28a or Compound 28b.

TABLE 3
iNeurons

| | % of level in iNeurons from Controls | | | |
|---|---|---|---|---|
| | | | sPD | sPD |
| | | sPD | patients | patients |
| | Untreated | patients | treated with | treated with |
| | sPD | treated with | Compound | Compound |
| Parameter | patients | UDCA | 28a | 28b |
| ATP normalisation | 50 | 82 | 89.6 | 94.3 |
| (% of controls) | | | | |
| ATP EC₅₀ (nM) | | 5 | 6.5 | 5 |
| MMP normalisation | 52 | 73 | 71 | 76 |
| (% of controls) | | | | |
| MMP EC₅₀ (nM) | | 7.3 | 10.3 | 8.2 |
| ROS normalisation | 115 | 110 | 111 | 109 |
| (% of controls) | | | | |
| Neuronal morphology | 48 | 64 | Not tested | 68 |
| (elongation; % controls) | | | | |
| Activated caspase 3 levels | 162 | 143 | Not tested | 138 |

The data in Table 3 show very clearly that Compound 28b provides a protective effect on both mitochondrial parameters in sPD derived dopaminergic neurons in addition to improving neuronal morphology and reducing apoptosis levels (as measured by activated caspase 3 levels). Apoptosis is a major mechanism of cell death of the dopaminergic neurons in culture and of dopaminergic neurons in patients with PD.

In Vivo Mouse Data with Compound 28b.

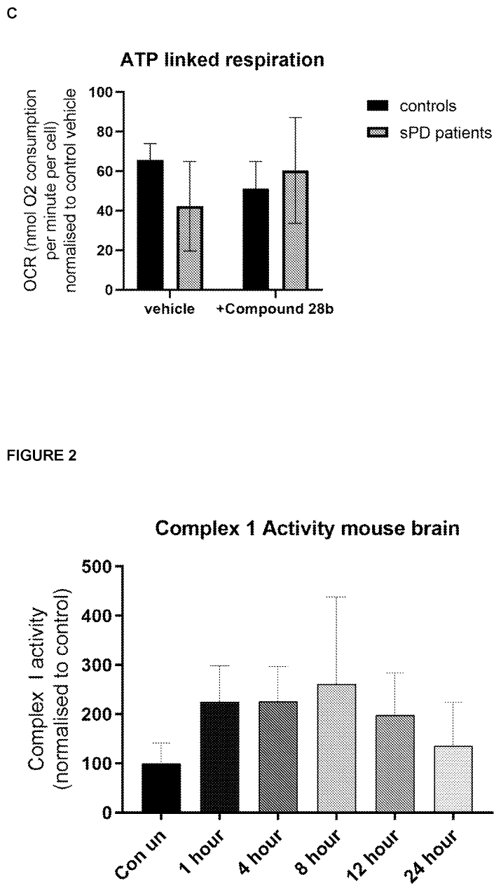

FIG. 2 shows that mitochondrial respiratory chain complex I activity is increased in wild type mouse whole brain homogenate after dosing with Compound 28b 2 mg/kg IV and collecting brain hemisphere samples (from 3 animals per group) at 1, 4, 8, 12 and 24 hours post injection. Complex I activity was increased 125% 1 hour after dosing, the levels remained elevated to a similar extent at 4 and 8 hours post injection. By 12 hours post injection complex I activity began to reduce again and was elevated by 98% above control. By 24 hours post injection, complex I activity was still elevated by 35% above control levels.

### Preliminary Assay of Compounds of Example 27

A preliminary assay with additional compounds of the invention was carried out to determine cellular ATP levels in fibroblasts from 3 patients with sporadic Parkinson's disease. The results are shown in Table 4.

TABLE 4
Cellular ATP levels data from 3 sporadic PD
patient fibroblasts in Preliminary ATP assay

| | Average ATP | Average ATP |
|---|---|---|
| | Response | Response |
| Compound | 100 nM | 1 μM |
| UDCA | 104.8 | 98.91 |
| 51a | 101.3 | 107.3 |
| 52a | 106.4 | 110.3 |
| 53a | 108.7 | 102.2 |
| UDCA | 104.8 | 98.91 |
| 54a | 109.2 | 107.4 |
| 55a | 109.7 | 109.0 |
| 56a | 109.4 | 100.1 |
| 57a | 108.6 | 106.7 |
| 58a | 102.2 | 102.57 |
| 59a | 106.1 | 99.71 |
| 60a | 101.7 | 102.5 |
| 61a | 104.7 | 107.9 |
| 62a | 108.3 | 108.3 |
| 63a | 106.3 | 103.8 |
| 64a | 118.2 | 111.4 |
| 65a | 110.7 | 108.4 |
| 66a | 109.1 | 111.0 |
| 67a | 108.1 | 107.2 |
| 68a | 108.6 | 99.01 |
| 69a | 106.8 | 102.48 |
| 70b | 105.68 | 106.9 |
| 71b | 105.8 | 103.7 |
| 72b | 101.5 | 112.4 |
| 73b | 99.61 | 100.4 |
| 74b | 102.8 | 98.5 |
| 75b | 112.4 | 105.6 |
| 76b | 104.8 | 106.5 |
| 77b | 109.2 | 113.2 |
| 50b | 101.2 | 99.8 |
| 78a | 120.6 | 120.1 |
| 79a | 116.0 | 121.6 |
| 80b | 121.9 | 120.4 |
| 81a | 94.15 | 102.5 |
| 82b | 96.15 | 97.45 |

NB: sPD fibroblasts have an average reduction of 18.33% of cellular ATP levels as compared to controls. Therefore a % of vehicle treated sPD fibroblasts of 118% or more is an increase to control ATP levels or higher than control ATP levels. Most compounds of the invention are more active at lower concentration ranges which may explain the drop off in activity seen at 1 μM versus 100 nM.

The data in Table 4 show that some compounds, for example Compounds 52a, 54a, 55a, 57a, 62a, 64a, 65a, 66a, 67a, 75b, 77b, 78a, 79a and 80b show particularly good recovery of ATP production across both concentrations, with Compound 64a at 100 nM returning ATP production to control levels. ATP is the cellular energy component and important for many processes required for the health of these cells, therefore increasing ATP production is key for prolonging the lifespan of neurons in neurodegenerative diseases. Since, as discussed above, mitochondrial dysfunction is also thought to play a role in acute radiation syndrome, myalgic encephalomyelitis and long COVID, increasing ATP function is also likely to be beneficial for treating these conditions.

### REFERENCES

D'Amore, C.; Di Leva, F. S.; Sepe, V.; Renga, B.; Del Gaudio, C.; D'Auria, M. V.; Zampella, A.; Fiorucci, F.; Limongelli, V. “Design, Synthesis, and Biological Evaluation of Potent Dual Agonists of Nuclear and Membrane Bile Acid Receptors”, J. Med. Chem. 57, 937-954 (2014)
S. M. Bell, K. Barnes, H. Clemmens, A. R. Al-Rafiah, E. A. Al-ofi, V. Leech, O. Bandmann, P. J. Shaw, D. J. Blackburn, L. Ferraiuolo, and H. Mortiboys, “Ursodeoxycholic Acid Improves Mitochondrial Function and Redistributes Drp1 in Fibroblasts from Patients with either Sporadic or Familial Alzheimer's Disease.” Journal of Molecular Biology, 430(21), 3942-3953 (2018). DOI: 10.1016/j.jmb.2018.08.019
Carling P J, Mortiboys H, Green C, Mihaylov S, Sandor C, Schwartzentruber A, Taylor R, Wei W, Hastings C, Wong S, Lo C, Evetts S, Clemmens H, Wyles M, Willcox S, Payne T, Hughes R, Ferraiuolo L, Webber C, Hide W, Wade-Martins R, Talbot K, Hu M T, Bandmann O. “Deep phenotyping of peripheral tissue facilitates mechanistic disease stratification in sporadic Parkinson's disease”. Prog Neurobiol., 187, 101772 (2020). doi: 10.1016/j.pneurobio.2020.101772.
S. D. Mhatre, J. Iyer, S. Puukila, A. M. Paul, C. G. T. Tahimic, L. Rubinstein, M. Lowe, J. S. Alwood, M. B. Sowa, S. Bhattacharya, R. K. Globus, and A. E. Ronca “Neuro-consequences of the spaceflight environment” Neuroscience and Biobehavioural Reviews, 132, 908-935 (2022)
H. Mortiboys, K. J. Thomas, W. J. H. Koopman, S. Klaffke, P. Abou-Sleiman, S. Olpin, N. W. Wood, P. H. G. M. Willems, J. A. M. Smeitink, M. R. Cookson, and O. Bandmann, “Mitochondrial function and morphology are impaired in parkin-mutant fibroblasts”, Ann Neurol. November; 64(5):555-65 (2008)
H. Mortiboys, J. Aasly, and O. Bandmann, “Ursocholanic acid rescues mitochondrial function in common forms of familial Parkinson's disease”, Brain, 136(10), 3038-3050 (2013)
H. Mortiboys, R. Furmston, G. Bronstad, J. Aasly, C. Elliott, and O. Bandmann, “UDCA exerts beneficial effect on mitochondrial dysfunction in LRRK2G2019S carriers and in vivo” Neurology, 85, 846-852 (2015)
Schwartzentruber A, Boschian C, Lopes F M, Myszczynska M A, New E J, Beyrath J, Smeitink J, Ferraiuolo L, Mortiboys H. “Oxidative switch drives mitophagy defects in dopaminergic parkin mutant patient neurons” Sci Rep, 10(1), 15485 (2020 Sep. 23). doi: 10.1038/s41598-020-72345-4
W. A. da Silveira, H. Fazelinia, S. Brin Rosenthal, E. C. Laiakis, M. S. Kim, C. Meydan, Y. Kidane, K. S. Rathi, S. M. Smith, B. Stear, Y. Ying, Y. Zhang, J. Foox, S. Zanello, B. Crucian, D. Wang, A. Nugent, H. A. Costa, S. R. Zwart, S. Schrepfer, R. A. L. Elworth, N. Sapoval, T. Treangen, M. MacKay, N. S. Gokhale, S. M. Horner, L. N. Singh, D. C. Wallace, J. S. Willey, J. C. Schisler, R. Meller, J. T. McDonald, K. M. Fisch, G. Hardiman, D. Taylor, C. E. Mason, S. V. Costes, and A. Beheshti, “Comprehensive Multi-omics Analysis Reveals Mitochondrial Stress as a Central Biological Hub for Spaceflight Impact”, Cell 183, 1185-1201, (2020)
E. Wood, K. H. Hall, and W. Tate “Role of mitochondria, oxidative stress and the response to antioxidants in myalgic encephalomyelitis/chronic fatigue syndrome: A possible approach to SARS-CoV-2 ‘long-haulers’?” Chronic Diseases and Translational Medicine, 7(1), 14-26 (2021)

What is claimed is:

1-93. (canceled)

94. A compound of formula (I): 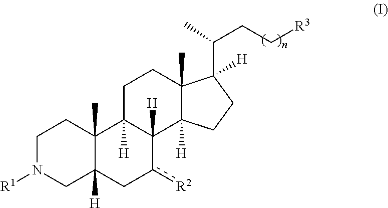
wherein:
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl; and
wherein each of R^{11a} and R^{11b} is independently selected from H and C₁₋₄ alkyl;
R² is selected from O and OH, wherein when R² is O, [symbol] is a double bond and when R² is OH, [symbol] is a single bond;
R³ is selected from C(O)OH, C(O)OR¹⁶, C(O)N(R⁶)—X¹—R⁷, C(O)N(R⁸)(R⁹) and C(O)S—R¹⁰;
R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently as defined above; and
n is 1 or 2;
or a salt or solvate thereof.

95. A compound, salt or solvate according to claim 94, wherein [symbol] is a single bond and the compound is a compound of formula (IA) or (IB): 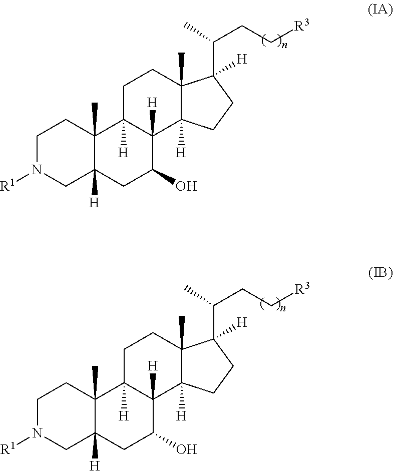
wherein
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl; and
wherein each of R^{11a} and R^{11b} is independently selected from H and C₁₋₄ alkyl;
R³ is selected from C(O)OH, C(O)OR¹⁶, C(O)N(R⁶)—X¹—R⁷, C(O)N(R⁸)(R⁹) and C(O)S—R¹⁰;
R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently as defined above; and
n is 1 or 2.

96. A compound, salt or solvate according to claim 94, wherein [symbol] is a double bond and the compound is a compound of formula (IC): 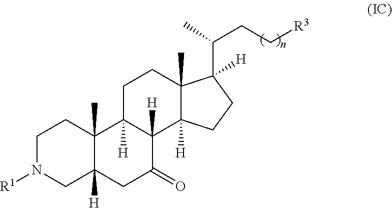
wherein
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl; and
wherein each of R^{11a} and R^{11b} is independently selected from H and C₁₋₄ alkyl;
R³ is selected from C(O)OH, C(O)OR¹⁶, C(O)N(R⁶)—X¹—R⁷, C(O)N(R⁸)(R⁹) and C(O)S—R¹⁰;
R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}) N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
R⁸ is selected from H, C₁₋₆ alkyl and a 3- to 6-membered carbocyclyl group optionally substituted with one or more substituents selected from C₁₋₅ alkyl, OH, O—(C₁₋₅ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₅ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently as defined above; and
n is 1 or 2.

97. A compound, salt or solvate according to claim 94, wherein R¹ is H, methyl or ethyl.

98. A compound, salt or solvate according to claim 94, wherein R¹ is —C(O)R⁴, wherein R⁴ is selected from C₁₋₆ alkyl optionally substituted with one or more substituents selected from OH, NH₂, NH₃′, phenyl optionally substituted with one or more substituents selected from OH and halo, and a nitrogen-containing heteroaryl group selected from pyrrole, pyridine and indole, optionally substituted with one or more substituents selected from OH and halo.

99. A compound, salt or solvate according to claim 98, wherein R¹ is an amino acid residue selected from residues of glycine, valine, isoleucine, leucine, tryptophan and tyrosine and salts of these amino acid residues.

100. A compound, salt or solvate according to claim 94, wherein R¹ is —C(O)OR⁵ wherein R⁵ is selected from C₁₋₆ alkyl, benzyl or fluorenylmethyl.

101. A compound, salt or solvate according to claim 94, which is a salt of formula (ID), (IE), (IF) or (IG): 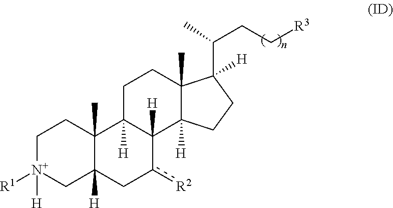 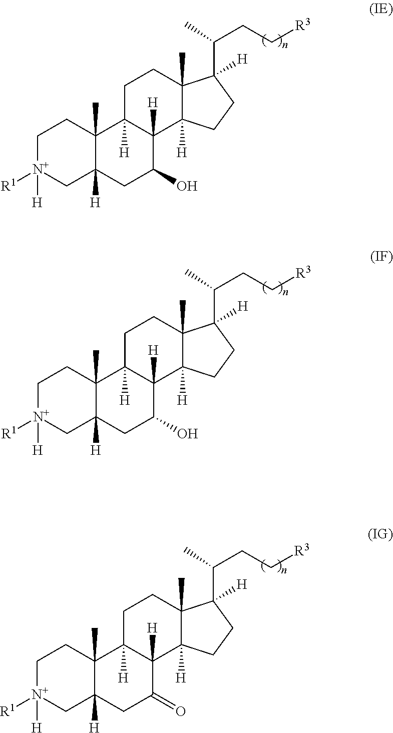
wherein:
R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl; and
wherein each of R^{11a} and R^{11b} is independently selected from H and C₁₋₄ alkyl;
R² is selected from O and OH, wherein when R² is O, [symbol] is a double bond and when R² is OH, [symbol] is a single bond;
R³ is selected from C(O)OH, C(O)OR¹⁶, C(O)N(R⁶)—X¹—R⁷, C(O)N(R⁸)(R⁹) and C(O)S—R¹⁰;
R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently as defined above; and
n is 1 or 2;
wherein the salt comprises a counterion Z⁻ selected from chloride, trifluoroacetate, mesylate, bromide, sulphate, and fumarate is present.

102. A compound, salt or solvate according to claim 94, wherein n is 1.

103. A compound, salt or solvate according to claim 94, wherein n is 2.

104. A compound, salt or solvate according to claim 94, wherein R³ is C(O)OH or C(O)O(C₁₋₄ alkyl).

105. A compound, salt or solvate according to claim 94, wherein R³ is C(O)N(R⁶)—X¹—R⁷, wherein R⁶ is H or methyl; and wherein X¹ is unsubstituted or substituted with one or more substituents selected from fluoro, OH, methoxy, ethoxy, i-propyloxy, s-butyloxy, t-butyloxy, S-methyl, NH₂, C(O)OH, phenyl and phenyl substituted with OH; and
wherein R⁷ is selected from C(O)OH, C(O)O—(C₁₋₃ alkyl) and S(O)₂OH.

106. A compound, salt or solvate according to claim 94 wherein R³ is C(O)N(R⁸)(R⁹), where:
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo.

107. A compound, salt or solvate according to claim 106, wherein R⁸ is selected from H, methyl, ethyl, unsubstituted cyclopentyl and unsubstituted cyclohexyl.

108. A compound, salt or solvate according to claim 106, wherein R⁸ is H and R⁹ is methyl, which is unsubstituted or substituted with a 5- or 6-membered heterocyclyl group selected from morpholinyl, piperidinyl, piperazinyl, pyrrolidinyl and tetrahydrofuryl, wherein the heterocyclyl group is unsubstituted or substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl).

109. A compound, salt or solvate according to claim 106, wherein R⁹ is selected from:
a carbocyclyl group selected from cyclopentyl and cyclohexyl; a heterocyclyl group selected from pyrrolidinyl, tetrahydrofuranyl, piperidinyl, piperazinyl and morpholinyl wherein the heterocyclyl group is unsubstituted or substituted with oxo;
phenyl or naphthyl wherein phenyl or naphthyl is unsubstituted or substituted with one or more substituents selected from halo, C(O)OH and C(O)O—(C₁₋₄ alkyl); and
pyridyl which is unsubstituted or substituted with one or more substituents selected from halo, C(O)OH and C(O)O—(C₁₋₄ alkyl).

110. A compound, salt or solvate according to claim 109, wherein R⁸ is H.

111. A compound, salt or solvate according to claim 109, wherein R⁸ and R⁹ together with the nitrogen atom to which they are attached combine to form a 4- to 10-membered heterocyclic group wherein:
the heterocyclic group is selected from piperidine, pyrrolidine, piperazine, morpholine and isothiazolidine, and is unsubstituted or is substituted as defined in claim 94; or
wherein the heterocyclic group comprises two rings, which are fused or bridged or joined by a spiro linkage; or
wherein the heterocyclic group is a piperidone or a pyrrolidone ring.

112. A compound, salt or solvate according to claim 109, wherein R⁸ and R⁹ together with the nitrogen atom to which they are attached combine to form a 5- to 10-membered heteroaryl group optionally containing one or more further heteroatoms selected from N, O and S, wherein the heteroaryl group is unsubstituted or is substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)OH and C(O)O—(C₁₋₆ alkyl).

113. A compound, salt or solvate according to claim 94, wherein R³ is C(O)S—R¹⁰, where R¹⁰ is selected from C₁₋₆ alkyl optionally substituted with OH, halo or phenyl.

114. A compound, salt or solvate according to claim 94, wherein R² is OH, R³ is C(O)OH and the compound is of formula (IH) or a salt of formula (IJ): 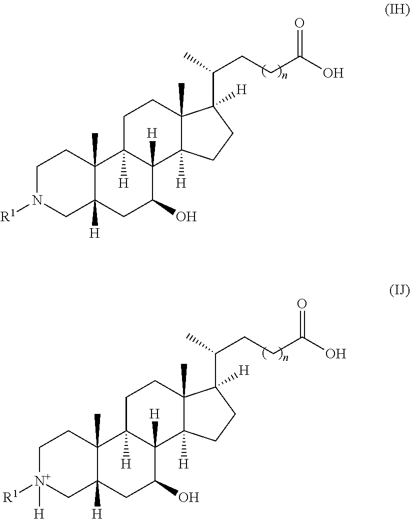
where R¹ is selected from H, C₁₋₆ alkyl, C₂₋₆ alkenyl, C₂₋₆ alkynyl, C(O)R⁴ and —C(O)OR⁵, wherein alkyl, alkenyl and alkynyl R¹ groups are optionally substituted with one or more substituents independently selected from OR^{11a} and N(R^{11a})(R^{11b});
wherein each of R⁴ and R⁵ is independently C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl; and
wherein each of R^{11a} and R^{11b} is independently selected from H and C₁₋₄ alkyl;
or
wherein R¹ is H, R² is OH and n is 1 and the compound is of formula (IK) or a salt of formula (IL) 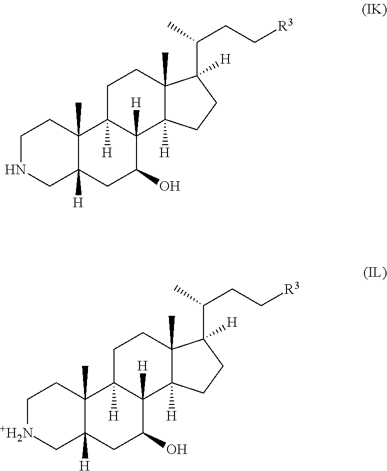
where R³ is selected from C(O)OH, C(O)OR¹⁶, C(O)N(R⁶)—X¹—R⁷, C(O)N(R⁸)(R⁹) and C(O)S—R¹⁰;
R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₆ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently as defined above.

115. A compound according to claim 94, selected from:
tert-Butyl N-(benzyloxycarbony)-3-aza-7β-hydroxy-5β-cholan-24-oate (25a);
tert-Butyl 3-aza-7β-hydroxy-5β-cholan-24-oate (27a);
3-Aza-7β-hydroxy-5β-cholan-24-oic acid (28a);
tert-Butyl N-methyl-3-aza-7β-hydroxy-5β-cholan-24-oate (29a);
N-Methyl-3-aza-7β-hydroxy-5β-cholan-24-oic acid (31a and 33);
tert-Butyl N-ethyl-3-aza-7β-hydroxy-5β-cholan-24-oate (30);
N-Ethyl-3-aza-7β-hydroxy-5β-cholan-24-oic acid (32);
tert-Butyl N-glycolyl-3-aza-7β-hydroxy-5β-cholan-24-oate (34a);
N-Glycolyl-3-aza-7β-hydroxy-5β-cholan-24-oic acid (40a);
N-[(2S)-2-amino-3-methylbutanoyl]-3-aza-7β-hydroxy-5β-cholan-24-oic acid (41a);
N-[(2S,3S)-2-Amino-3-methylpentanoyl]-3-aza-7β-hydroxy-5β-cholan-24-oic acid (42a);
N-[(2S)-2-Amino-4-methylpentanoyl]-3-aza-7β-hydroxy-5β-cholan-24-oic acid (43a);
N-[(2S)-2-Amino-3-(1H-indol-3-yl)propanoyl]-3-aza-7β-hydroxy-5β-cholan-24-oic acid (44a);
N-[(2S)-2-Amino-3-(4-hydroxyphenyl)propanoyl]-3-aza-7β-hydroxy-5β-cholan-24-oic acid (45a);
tert-Butyl N-(benzyloxycarbonyl)-3-aza-7β-hydroxy-25-homo-5β-cholan-25-oate (25b);
tert-Butyl 3-aza-7β-hydroxy-25-homo-5β-cholan-25-oate (27b);
3-Aza-7β-hydroxy-25-homo-5β-cholan-25-oic acid (28b);
tert-Butyl N-methyl-3-aza-7β-hydroxy-25-homo-5β-cholan-25-oate (29b);
N-Methyl-3-aza-7β-hydroxy-25-homo-5β-cholan-25-oic acid (31b);
tert-Butyl N-glycolyl-3-aza-7β-hydroxy-25-homo-5β-cholan-25-oate (34b);
N-Glycolyl-3-aza-7β-hydroxy-25-homo-5β-cholan-25-oic acid (40b);
N-[(2S)-2-Amino-3-methylbutanoyl]-3-aza-7β-hydroxy-25-homo-5β-cholan-25-oic acid (41b);
N-[(2S,3S)-2-Amino-3-methylpentanoyl]-3-aza-7β-hydroxy-25-homo-5β-cholan-25-oic acid (42b);
N-[(2S)-2-Amino-4-methylpentanoyl]-3-aza-7β-hydroxy-25-homo-5β-cholan-25-oic acid (43b, Example 25);
N-[(2S)-2-Amino-3-(1H-indol-3-yl)propanoyl]-3-aza-7β-hydroxy-25-homo-5β-cholan-25-oic acid (44b);
N-(benzyloxycarbony)-3-aza-7β-hydroxy-5β-cholan-24-oic acid (50a);
N-(3-Aza-7β-hydroxy-5β-cholan-24-amide)-ethylsulfonic acid (51a);
N-(3-Aza-7β-hydroxy-5β-cholan-24-amide)-acetic acid (52a);
N-(3-Methyl-aza-7β-hydroxy-5β-cholan-24-oyl)-(2S)-2-amino-3-[(2-methylpropan-2-yl)oxy]propanoic acid (53a);
N-(3-Methyl-aza-7β-hydroxy-5β-cholan-24-oyl)-(R)-3-amino-3-phenylpropanoic acid (54a);
N-(3-Methyl-aza-7β-hydroxy-5β-cholan-24-oyl)-1-amino-4-fluorobenzene (55a);
N-(3-Aza-7β-hydroxy-5β-cholan-24-oyl)-morpholine (56a);
N-(3-Aza-7β-hydroxy-5β-cholan-24-oyl)-(S)-2-amino-3-hydroxypropanoic acid (57a);
N-(3-Aza-7β-hydroxy-5β-cholan-24-oyl)-3-amino-2-fluoropropanoic acid (58a);
N-(Cyclohexyl)-N-(3-methyl-aza-7β-hydroxy-5β-cholan-24-oyl)-cyclohexanamine (59a);
N-(3-Aza-7β-hydroxy-5β-cholan-24-oyl)-4-aminobenzoic acid (60a);
N-(3-Aza-7β-hydroxy-5β-cholan-24-oyl)-(S)-2-amino-4-(methylthio)butanoic acid (61a);
N-(3-Aza-7β-hydroxy-5β-cholan-24-amide)-propanoic acid (62a);
N-(3-Aza-7β-hydroxy-5β-cholan-24-oyl)-(isopropyl-4-aminobenzoate) (63a);
S-(3-Aza-7β-hydroxy-5β-cholan-24-oyl)-phenylmethanethiol (64a);
N-(3-Aza-7β-hydroxy-5β-cholan-24-oyl)-(S)-2-amino-3-methylbutanoic acid (65a);
N-(3-Aza-7β-hydroxy-5β-cholan-24-oyl)-(2S,3S)-2-amino-3-methylpentanoic acid (66a);
N-Methyl-N-(3-aza-7β-hydroxy-5β-cholan-24-oyl)-glycine (67a);
N-(3-Aza-7β-hydroxy-5β-cholan-24-oyl)-(S)-2-aminopropanoic acid (68a);
N-(benzyloxycarbonyl)-3-aza-7β-hydroxy-24-homo-5β-cholan-25-oic acid (50b);
N-(3-Aza-7β-hydroxy-27-homo-5β-cholan-27-oyl)-(S)-2-aminobutanedioic acid (70b);
N-(3-Aza-7β-hydroxy-27-homo-5β-cholan-27-oyl)-(2S)-2-amino-3-[(2-methylpropan-2-yl)oxy]propanoic acid (71b);
N-(3-Aza-7β-hydroxy-27-homo-5β-cholan-27-amide)-ethylsulfonic acid (72b);
N-(3-Aza-7β-hydroxy-27-homo-5β-cholan-27-oyl)-(S)-2-amino-3-phenylpropanoic acid (73b);
N-(3-Aza-7β-hydroxy-27-homo-5β-cholan-27-oyl)-3-aminobutanoic acid (74b);
N-(3-Aza-7β-hydroxy-27-homo-5β-cholan-27-oyl)-(S)-2-amino-4-methylpentanoic acid (75b);
N-(3-Aza-7β-hydroxy-27-homo-5β-cholan-27-oyl)-(2S)-2,6-diaminohexanoic acid (76b);
N-(3-Aza-7β-hydroxy-27-homo-5β-cholan-27-oyl)-(2S)-2-amino-3-(4-hydroxyphenyl)propanoic acid (77b);
N-{(benzyloxycarbonyl)-3-aza-7β-hydroxy-5β-cholan-24-oyl}-2-oxa-6-azospiro{3,3}heptane (78a);
N-{(benzyloxycarbonyl)-3-aza-7β-hydroxy-5β-cholan-24-oyl}-4-piperidone (79a);
N-{(benzyloxycarbonyl)-3-aza-7β-hydroxy-25-homo-5β-cholan-25-oyl}-3-aminotetrahydrofuran (80b);
N-{(3-aza-7β-hydroxy-5β-cholan-25-oyl}-isoindoline (81a);
N-{(3-aza-7β-hydroxy-25-homo-5β-cholan-25-oyl)}-3-aminotetrahydrofuran (82b); and
salts and solvates thereof.

116. A method of preparing a compound of formula (I) or a salt or solvate thereof according to claim 94, the method comprising:
A. for a compound of formula (I) in which R¹ is C(O)OR⁵ and R³ is C(O)OR¹⁶:
reacting a compound of formula (II): 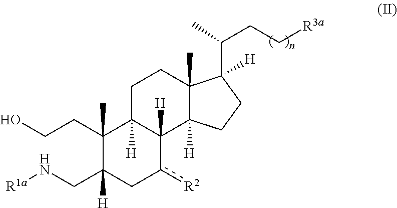
wherein
R² is selected from O and OH, wherein when R² is O, [symbol] is a double bond and when R² is OH, [symbol] is a single bond;
n is 1 or 2;
R^{1a} is C(O)OR⁵ wherein R⁵ is C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl); and
R^{14a} and R^{14b} are each independently selected from H and C₁₋₆ alkyl;
and R^{3a} is C(O)OR¹⁶, wherein R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl;
with methane sulfonyl chloride in an organic solvent at about −5° to 5° C.;
B. for a compound of formula (I) in which R¹ is H:
hydrogenation of a compound of formula (I) in which R¹ is C(O)OR⁵ over a palladium catalyst;
C. for a compound of formula (I) in which R¹ is C₁₋₆ alkyl and R³ is C(O)OR¹⁶:
reaction of a compound of formula (I) in which R¹ is H and R³ is C(O)OR¹⁶ with a compound of formula (XX):
R^{1b}—C(O)H (XX)
wherein R^{1b} is C₁₋₅ alkyl;
followed by hydrogenation over a palladium/carbon catalyst;
D. for a compound of formula (I) in which R¹ is C₂₋₆ alkenyl or C₂₋₆ alkynyl and R³ is C(O)OR¹⁶:
reaction compound of formula (I) in which R¹ is H and R³ is C(O)OR¹⁶ with a compound of formula (XVIII): 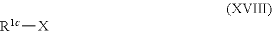
wherein R^{1c} is C₂₋₆ alkenyl or C₂₋₆ alkynyl and X is chloro or bromo;
in the presence of a base;
wherein when R² is OH, the method further comprises the initial step of protecting R² with a suitable protecting group and the final step of removing the protecting group;
E. for a compound in which R³ is C(O)OH:
acid or base hydrolysis of a compound of formula (I) in which R³ is C(O)OR¹⁶;
F. for a salt of formula (I) in which the nitrogen atom to which R¹ is attached is quaternised:
treatment of a compound of formula (I) in which R₁ is H with an acid.
G. for a compound of formula (I) in which R¹ is C(O)R⁴ and R³ is C(O)OR¹⁶:
i. reaction of a compound of formula (I) in which R¹ is H and R³ is C(O)OR¹⁶ with a compound of formula (XXI): 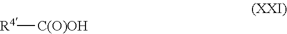
wherein R^{4′} is C₁₋₆ alkyl optionally substituted with one or more substituents selected from OR^{14a}, N(R^{14a})(R^{14b}), NH₃⁺, C(O)N(R^{14a})(R^{14b}), SR¹⁴ a protected OH group, a protected NH₂ group, a protected C(O)NH₂ group, a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl);
under basic conditions, and in the presence of a coupling reagent; and
ii. where necessary, removal of a protecting group to give a group R⁴ comprising an OH, NH₂ or C(O)NH₂;
H. for a compound of formula (I) in which R¹ is C(O)R⁴ and R³ is C(O)OH:
reaction of the product of G(i) with trifluoroacetic acid;
I. for a compound of formula (I) in which R³ is C(O)N(R⁶)—X¹—R⁷:
reaction of a compound of formula (I) in which R³ is C(O)OH with a compound of formula (XXV): 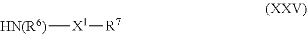
wherein
X¹ is C₁₋₆ alkylene optionally substituted with one or more substituents selected from halo, OR^{12a}, SR^{12a}, N(R^{12a})(R^{12b}) C(O)OR^{12a}, C(O)N(R^{12a})(R^{12b}), N(R^{12a})—C(NH)—N(R^{12a})(R^{12b}), N(R^{12a})—C(N⁺H₂)—N(R^{12a})(R^{12b}) and 6- to 14-membered aryl or 5- to 14-membered heteroaryl, wherein aryl and heteroaryl groups are optionally substituted with one or more substituents selected from halo, C₁₋₆ alkyl, C₁₋₆ haloalkyl OR^{13a}, N(R^{13a})(R^{13b}), NO₂, S(O)₂OH, and CN;
R^{12a} and R^{12b} are each independently selected from H and C₁₋₆ alkyl;
R^{13a} and R^{13b} are each independently selected from H, C₁₋₆ alkyl and C₁₋₆ haloalkyl;
R⁶ is selected from H, methyl and ethyl;
R⁷ is selected from C(O)OH, C(O)O—(C₁₋₆ alkyl), S(O)₂OH, and S(O)₂—O—(C₁₋₆ alkyl);
in the presence of a coupling reagent and under basic conditions, for example in the presence of an amine selected from diisopropylethylamine (DIPEA) or triethylamine (TEA) and in an organic solvent;
J. for a compound of formula (I) in which R³ is C(O)N(R⁸)(R⁹):
reaction of a compound formula (I) in which R³ is C(O)OH with a compound of formula (XXVI): 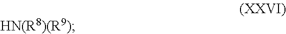
where:
R⁸ is selected from H, C₁₋₆ alkyl and a 3- to 6-membered carbocyclyl group optionally substituted with one or more substituents selected from C₁₋₆ alkyl, OH, O—(C₁₋₆ alkyl), N(R^{19a})(R^{19b}), C₁₋₆ haloalkyl and halo;
wherein R^{19a} and R^{19b} are each independently selected from H and C₁₋₆ alkyl;
R⁹ is selected from H, C₁₋₆ alkyl, a 3- to 7-membered carbocyclyl group, a 3- to 7-membered heterocyclyl group, 6- to 14-membered aryl and 5- to 14-membered heteroaryl;
wherein alkyl groups are optionally substituted with one or more substituents selected from C₁₋₄ alkyl, OH, O—(C₁₋₄ alkyl), C₁₋₄ haloalkyl, O—(C₁₋₄ haloalkyl), halo, N(R^{19a})(R^{19b}), phenyl, 3- to 7-membered carbocyclyl and 3- to 7-membered heterocyclyl;
wherein R^{19a} and R^{19b} are each independently as defined above;
wherein carbocyclyl and heterocyclyl groups are optionally substituted with one or more substituents selected from C₁₋₄ alkyl, OH, O—(C₁₋₄ alkyl), C₁₋₄ haloalkyl, O—(C₁₋₄ haloalkyl), oxo, phenyl, benzyl and halo, provided that heteroatoms of a heterocyclyl group are not substituted with OH, O—(C₁₋₄ alkyl) or O—(C₁₋₄ haloalkyl); and
wherein aryl and heteroaryl are optionally substituted with one or more substituents selected from halo, NO₂, CN, S(O)₂OH, C₁₋₆ alkyl, C₁₋₆ haloalkyl, O(R^{15a}), N(R^{15a})(R^{15b}), C(O)N(R^{15a})(R^{15b}), C(O)OH and C(O)O—(C₁₋₆ alkyl);
wherein R^{15a} and R^{15b} are each independently selected from H, C₁₋₆ alkyl and C₁₋₆ haloalkyl; or
R⁸ and R⁹ together with the nitrogen atom to which they are attached combine to form a 4- to 10-membered heterocyclic group, optionally containing one or more further heteroatoms selected from O, N and S and optionally substituted with one or more substituents selected from C₁₋₄ alkyl, OH, O—(C₁₋₄ alkyl), halo, C₁₋₄ haloalkyl, O—(C₁₋₄ haloalkyl), C(O)OH, C(O)O(C₁₋₄ alkyl), phenyl, benzyl, CN, N(R^{15a})(R^{15b}), C(O)N(R^{15a})(R^{15b}) and oxo provided that heteroatoms of a heterocyclyl group are not substituted with CN, N(R^{15a})(R^{15b}), OH, O—(C₁₋₄ alkyl) or O—(C₁₋₄ haloalkyl);
wherein alkyl groups are optionally substituted by one or more groups selected from O—(C₁₋₄ alkyl), O—(C₁₋₄ haloalkyl), N(R^{15a})(R^{15b}), OH and C₃₋₆ cycloalkyl; or
R⁸ and R⁹ together with the nitrogen atom to which they are attached combine to form a 5- to 10-membered heteroaryl group optionally containing one or more further heteroatoms selected from N, O and S and optionally substituted with one or more substituents selected halo, NO₂, CN, S(O)₂OH, C₁₋₆ alkyl, C₁₋₆ haloalkyl, O(R^{15a}), N(R^{15a})(R^{15b}), C(O)OH, C(O)N(R^{15a})(R^{15b}) and C(O)O—(C₁₋₅ alkyl);
wherein R^{15a} and R^{15b} are each independently as defined above;
wherein alkyl groups are optionally substituted by one or more groups selected from OH and C₃₋₆ cycloalkyl;
wherein when the heteroaryl group contains a non-aromatic ring, the non-aromatic ring may also be substituted with oxo;
under basic conditions and in the presence of a coupling agent;
K. for a compound of formula (I) in which R⁸ and R⁹ together with the nitrogen atom to which they are attached combine to form a 5-membered heteroaryl ring:
coupling reaction of a compound of formula (I) in which R³ is C(O)N(R⁸)(R⁹), where R⁸ and R⁹ together with the nitrogen atom to which they are attached combine to form a 5- or 6-membered heterocyclic ring substituted with oxo;
L. for a compound of formula (I) in which R³ is C(O)S—R¹⁰:
reaction of a compound formula (I) in which R³ is C(O)OH with a compound of formula (XXVII): 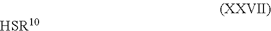
where R¹⁰ is C₁₋₆ alkyl optionally substituted with OH, halo or phenyl, wherein phenyl is optionally substituted with one or more substituents selected from halo, NO₂, CN, S(O)₂OH, C₁₋₆ alkyl, C₁₋₆ haloalkyl, O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl.

117. A method for the treatment or prevention of a neurodegenerative disorder, acute radiation syndrome or myalgic encephalomyelitis (ME, chronic fatigue syndrome) or post viral syndrome, including chronic symptoms arising from infection with SARS-CoV2 (long COVID), the method comprising administering to a patient in need of such treatment an effective amount of a compound, salt or solvate according to claim 94.

118. A pharmaceutical composition comprising a compound, salt or solvate according to claim 94, and a pharmaceutically acceptable excipient or carrier.

119. A pharmaceutical composition according to claim 118, formulated for parenteral administration, oral administration topical administration to the skin (transdermal administration) or topical administration to the lung (by inhalation).

120. A compound of formula (II): 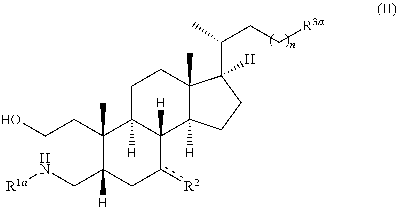
wherein R² is selected from O and OH, wherein when R² is O, [symbol] is a double bond and when R² is OH, [symbol] is a single bond, n is 1 or 2, R^{1a} is C(O)OR⁵ and R^{3a} is C(O)OR¹⁶, wherein R⁵ is C₁₋₆ alkyl optionally substituted with one or more substituents selected from OR^{14a}, N(R^{14a})(R^{14b}), NH₃⁺, C(O)N(R^{14a})(R^{14b}), SR¹⁴ a 5- or 6-membered nitrogen-containing heterocyclic ring and a 6- to 14-membered aryl or 5- to 14-membered heteroaryl, wherein the aryl and heteroaryl are optionally substituted with one or more substituents selected from OH, halo, NH₂, NO₂, S(O)₂OH, C₁₋₆ alkyl, C₁₋₆ haloalkyl, O(C₁₋₆ alkyl) and O(C₁₋₆ haloalkyl, and R¹⁶ is selected from C₁₋₈ alkyl optionally substituted with one or more substituents selected from OH, halo and phenyl, wherein phenyl is optionally substituted with one or more substituents selected from halo, NO₂, CN, S(O)₂OH, C₁₋₆ alkyl, C₁₋₆ haloalkyl, O(C₁₋₆ alkyl) O(C₁₋₆ haloalkyl), O(R^{15a}), N(R^{15a})(R^{15b}) and C(O)N(R^{15a})(R^{15b});
wherein R^{15a} and R^{15b} are each independently selected from H, C₁₋₆ alkyl and C₁₋₆ haloalkyl; or
a compound of formula (IP2): 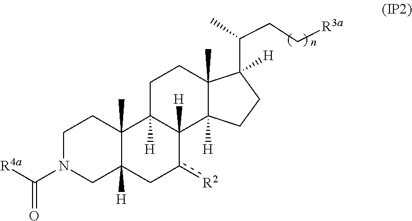
wherein R² is selected from O and OH, wherein when R² is O, [symbol] is a double bond and when R² is OH, [symbol] is a single bond, n is 1 or 2, R^{3a} is as defined for formula (II) above and R^{4a} is C₁₋₆ alkyl substituted with a protected NH₂ group, a protected OH group or a protected C(O)NH₂ group.
